(19) United States
(12) Patent Application Publication
(10) Pub. No.: US 2011/0086796 A1
(43) Pub. Date: Apr. 14, 2011

(54) METHODS FOR PREDICTING THE DEVELOPMENT AND RESOLUTION OF ACUTE RESPIRATORY DISTRESS SYNDROME

(75) Inventors: ZHAOXI WANG, CHELMSFORD, MA (US); DOUGLAS BEACH, BROOKLINE, MA (US); LI SU, SOMERVILLE, MA (US); RIHONG ZHAI, QUINCY, MA (US); DAVID C. CHRISTIANI, JAMAICA PLAIN, MA (US)

(73) Assignee: HARVARD UNIVERSITY, Cambridge, MA (US)

(21) Appl. No.: 12/772,639

(22) Filed: May 3, 2010

Related U.S. Application Data
(63) Continuation of application No. PCT/US2008/082287, filed on Nov. 3, 2008.
(60) Provisional application No. 60/984,856, filed on Nov. 2, 2007.
(60) Provisional application No. 61/054,414, filed on May 19, 2008.

(51) Int. Cl. A61K 38/55; C07H 21/00; A61P 11/00; C12Q 1/68; G01N 33/68; C12Q 1/37; C12Q 1/02

(52) U.S. Cl. 514/1.5; 536/25.6; 435/6; 436/86; 435/23; 435/29

(57) ABSTRACT

The subject invention features methods for predicting whether a subject at risk of developing Acute Respiratory Distress Syndrome (ARDS) will develop ARDS by determining the amount of elafin present in a subject sample, or by determining the ration of elafin:neutrophil elastase in a subject sample. The invention also features methods for monitoring the efficacy of a treatment regimen for ARDS as well as methods of treatment for ARDS. The invention also features methods to determine a subject's predisposition for developing ARDS by determining whether certain genomic polymorphisms are present in the subject's DNA.

### CROSS-REFERENCE TO RELATED APPLICATIONS

This application is a continuation of PCT/US2008/082287 filed on Nov. 3, 2008, and is related and claims priority to U.S. provisional application Ser. No. 60/984,856 filed on Nov. 2, 2007, and U.S. provisional application Ser. No. 61/054,414, filed May 19, 2008, the entire contents of each of the foregoing applications are incorporated herein by this reference.

### STATEMENT REGARDING FEDERALLY SPONSORED RESEARCH OR DEVELOPMENT

This invention was made with Government support awarded by the National Institutes of Health grants HL60710 and ES00002. The government has certain rights in the invention.

### BACKGROUND OF THE INVENTION

Acute Respiratory Distress Syndrome (ARDS), also called Adult Respiratory Distress Syndrome, is not a specific disease, but a lung dysfunction that results from a variety of diseases or conditions. ARDS is characterized by intense inflammatory responses to direct or indirect lung injury exposures, resulting in diffuse alveolar damage and severe, life threatening hypoxia (Goodman, R. B. et al. 1996. Am J Respir Crit Care Med 154:602-611; Holter, J. F. et al. 1986. J Clin Invest 78:1513-1522; Weiland, J. E., et al. 1986. Am Rev Respir Dis 133:218-225; Strieter, R. M., et al. 1999. Chest 116:103 S-110S.1-4). Risk factors for the development of ARDS include conditions commonly observed in critically ill patients, such as sepsis, trauma, pneumonia, burns, and massive transfusions of packed red blood cells. Direct lung injury by, for example, inhalation of toxins (such as smoke, chemical fumes, acids, stomach acids, etc.), by a severe blow to the chest, or severe pneumonia, drug overdose, or pancreatitis may also cause ARDS. In addition, infection with various viruses, such as the coronavirus known to cause severe acute respiratory syndrome (SARS) and the H5N1 avian influenza virus, may also lead to the development of ARDS (Brundage, J. F. 2006. Lancet Infect Dis 6:303-312; Chen, C. Y., et al. 2005. J Chin Med Assoc 68:4-10; and Looney, M. R. 2006. Clin Chest Med 27:591-600; abstract viii.). Of all of these risk factors, sepsis indicates the highest risk (˜40%) of developing ARDS.

Despite the broad range of risk factors, predicting the development of and monitoring the progression of ARDS is complex. One scoring system has been used widely to diagnose ARDS but cannot be used to predict which patients will develop ARDS or predict the outcome of a patient diagnosed with ARDS during the first 24 to 72 hours after the onset of ARDS (Doyle R L, et al. 1995. Am J Respir Crit Care Med. 152:1818-24; Zilberberg M D and Epstein S K. 1998. Am J Respir Crit Care Med. 157:1159-64). The most recent American-European Consensus Conference Committee (AECCC) criteria for ARDS, which uses the ratio of the partial pressure of arterial oxygen to the fraction of inspired oxygen to diagnose ARDS, is also a poor predictor of outcome. Furthermore, patients diagnosed with ARDS have few treatment options, typically, only supportive care is given.

Various estimates have indicated that the incidence of ARDS, range from 1.5 cases per 100,000 people to as high as 75 per 100,000. This is a significant number considering that the mortality rate for ARDS is reported to be 40%-60%. Thus, there is a clear need for definitive and rigorous methods to diagnose, monitor progression, and treat ARDS.

### SUMMARY OF THE INVENTION

The present invention is based, at least in part, on the discovery that prior to the onset of ARDS (e.g., the onset of the acute phase of ARDS; e.g., prior to clinical diagnosis), the amount of elafin is significantly increased in a biological sample derived from a subject (e.g., a subject at risk of developing ARDS and who subsequently develops or is diagnosed with ARDS; e.g., a subject who has a prior history of ARDS), as compared to the amount of elafin in a biological sample from a control subject (e.g., a healthy subject; e.g., a subject at risk of developing ARDS who does not develop ARDS and/or has no prior history of ARDS).

Furthermore, the present invention is based, at least in part, on the discovery that the amount of elafin is significantly decreased in a biological sample derived from a subject that is in the recovery phase of ARDS (e.g., has received an efficacious treatment), compared to the amount of elafin in a biological sample from a subject with ARDS (e.g., in the acute phase of ARDS) or from a subject developing ARDS.

The present invention is also based, at least in part, on the discovery that the ratio between elafin and human neutrophil elastase is decreased in a biological sample from a subject with the onset of ARDS.

In addition, the present invention is based, at least in part, on the discovery that certain nucleotide polymorphisms in the elafin gene are associated with an increased risk for developing ARDS.

Accordingly, the present invention provides a method of identifying a subject that is at high risk of developing ARDS. The method includes determining the amount of elafin present in a test sample from the subject; and comparing the foregoing amount of elafin to the amount of elafin present in a control sample, wherein an increased amount of elafin in the test sample relative to the amount in the control sample indicates that the subject is at high risk of developing ARDS.

In a related aspect, the present invention provides a method of identifying a subject that is at high risk of developing ARDS by determining the ratio between elafin and HNE in a test sample from the subject; and comparing the foregoing ratio to the ratio between elafin and HNE in a control sample, wherein a decrease in the elafin/HNE ratio indicates that the subject is at high risk of developing ARDS. Alternatively, the HNE/elafin ratios may be used, in which case an increase in the HNE/elafin ratio indicates that the subject is at high risk of developing ARDS.

A further aspect of the invention provides a method of monitoring clinical progress of a subject who is at high risk of developing Acute Respiratory Distress Syndrome (ARDS) the method comprising: determining the amount of elafin present in a first test sample from the subject; determining the amount of elafin present in a second test sample from the subject taken at a later time; and comparing the amount of elafin in the first test sample to the amount of elafin present in the second test sample, wherein a change in the amount of elafin between the first and the second test samples indicates a change in the clinical progression of ARDS. In some embodiments a decrease in the amount of elafin between the two test samples indicates a progression towards ARDS. In other embodiments, an increase in the amount of elafin between the two test samples indicates that further monitoring is required (e.g., an increase in plasma elafin levels in a pre-diagnosis subject indicates that the subject is at high risk of developing ARDS whereas, in post-diagnosis subjects, an increase in elafin levels indicates that further monitoring is needed).

A further embodiment of the invention provides a method of monitoring clinical progress of a subject who is at high risk of developing Acute Respiratory Distress Syndrome (ARDS) the method comprising: determining the ratio of elafin:neutrophil elastase present in a first test sample from the subject; determining the ratio of elafin:neutrophil elastase present in a second test sample from the subject taken at a later time; and comparing the ratio of elafin:neutrophil elastase in the first test sample to the ratio of elafin:neutrophil elastase present in the second test sample, wherein a decrease in the wherein an decreased ratio of elafin:neutrophil elastase indicates that the subject is at high risk of developing ARDS. In other embodiments, an increase in the ratio of elafin:neutrophil elastase between the two test samples indicates that further monitoring is required. In pre-diagnosis subjects, and decrease in plasma elafin/HNE ratio indicates that the subject is at high risk of developing ARDS. In post-diagnosis subjects, a steady level of the elafin/HNE ratio indicates that further monitoring is needed.

In some embodiments where samples are taken from a subject at more than one time point, such samples may be taken at any interval which permits detection of a change in clinical status. For example, the first and second test samples may be taken at least about 30 minutes, 45 minutes, 1 hour, 1.5 hours, 2 hours, 3 hours, 4 hours, 5 hours, 6 hours, 7 hours, 8 hours, 9 hours, 10 hours, 12 hours, 16 hours, 18 hours, 20 hours, 1 day, or at least about 30 hours apart. Indeed, such test samples may be taken at any interval between 15 minutes and 3 days, between 15 minutes and 1 day, between 30 minutes and 24 hours, 1 hour and 24 hours, or 1 hour and 12 hours. In some embodiments, the methods further comprise determining the amount of elafin and/or the elafin/HNE ratio at additional time points (e.g., 1, 2, 3, 4, 5, 6, 7, 8, 9, 10, 12, 15 or more time points). It will be appreciated by one of skill in the art that the intervals between the time points may vary, e.g., the interval between the first and second measurement may be 1 hour, and the interval between the second and third measurements may be 2 hours, etc.

In another aspect, the present invention provides a method of monitoring the efficacy of a treatment regimen for a subject with ARDS. In one embodiment, the method includes determining the amount of elafin present in a first test sample derived from a subject at a first point in time; determining the amount of elafin present in a second test sample derived from a subject at a second point in time after treatment has begun; and comparing the amount of elafin determined at each point in time, wherein a decrease in the amount of elafin determined at the second time point compared to the amount of elafin determined at the first time point indicates that a treatment is efficacious for treating ARDS. In another embodiment, the method includes determining the elafin/HNE ratio present in a first test sample derived from a subject at a first point in time; determining the elafin/HNE ratio present in a second test sample derived from a subject at a second point in time after treatment has begun; and comparing the elafin/HNE ratio determined at each point in time, wherein a increase in the ratio determined at the second time point compared to the ratio determined at the first time point indicates that a treatment is efficacious for treating ARDS. Alternatively, the HNE/elafin ratios may be used, in which case a decrease in the ratio determined at the second time point compared to the ratio determined at the first time point indicates that a treatment is efficacious for treating ARDS.

In another embodiment, the method includes determining the amount of HNE present in a first test sample derived from a subject at a first point in time; determining the amount of HNE present in a second test sample derived from a subject at a second point in time after treatment has begun; and comparing the amount of HNE determined at each point in time, wherein a decrease in the amount of HNE determined at the second time point compared to the amount of HNE determined at the first time point indicates that a treatment is efficacious for treating ARDS.

In certain embodiments genes which have been found to be differentially expressed at different stages of ARDS development may be used to determine the progression of the disease or to predict progression of the disease. For example, any one of the genes, or any combination of genes identified in Tables 2, 5, and 6 may be used to assess or predict the progression of ARDS.

In certain embodiments of the methods of the invention, the amount of elafin and/or HNE in the test sample is determined in vitro, e.g., in a tissue sample or a body fluid obtained from the subject such as, but not limited to, whole blood, serum, plasma, buccal scrape, saliva, cerebrospinal fluid, urine, stool, bronchioalveolar lavage, and lung tissue. In other embodiments, the level of elafin and/or HNE in the test sample is determined in vivo, e.g., using imaging methods.

In related embodiments, the amount of elafin protein and/or HNE protein in the test sample is determined. For example, the determination of the amount of elafin protein and/or HNE protein may be determined using an antibody having specificity for elafin protein or the HNE protein by immunoassay such as, but not limited to ELISA, Western blot, and mass spectrometry.

In other related embodiments, the amount of elafin nucleic acid and/or HNE nucleic acid in the test sample is determined. For example, the amount of elafin nucleic acid and/or HNE nucleic acid may be determined using a nucleic acid probe or primer capable of specifically hybridizing to elafin nucleic acid or the HNE nucleic acid using recombinant methods such as, but not limited to quantitative PCR, Northern blot, and expression array analysis.

In other embodiments of the methods of the invention, the treatment regimes which may be monitored according to the methods of the invention include, but are not limited to nutritional support, administration of sedatives, administration of analgesics, treatment of secondary conditions such as pneumonia, sepsis, or organ failure, control of blood glucose levels, administration of surfactants, administration of vasodilators, and administration of glucocorticoids.

In yet other embodiments, the methods for invention may further include one or more clinical tests for ARDS including, but not limited to, red blood cell count, blood gas measurement, blood pressure measurement, chest auscultation, chest radiograph, assessment of cyanosis of lips and/or nailbeds, and measurement of breathing frequency.

In still yet other embodiments, the subject is a human. In related embodiments, the subject has a condition associated with the development of ARDS including, but not limited to sepsis, trauma, pneumonia, burns, direct lung injury (e.g., inhalation of toxins including, but not limited to, smoke, chemical fumes, acids, particulates), chest trauma, pancreatitis, drug overdose, virus infection, or recent transfusion. In one preferred embodiment, the subject has sepsis.

In a further aspect, the invention provides a method for determining the predisposition of a subject to develop ARDS. The method includes detecting the presence of at least one single nucleotide polymorphism (SNP) in the subject wherein the presence of the SNP is associated with an increased risk of ARDS. In particular embodiments, the SNP is a polymorphism near or in the elafin gene such as, but not limited to the polymorphisms A959C, A162T, or A751T. In a preferred embodiment, the polymorphism is in a human elafin gene. In some embodiments the polymorphism is a polymorphism identified in Table 12 or FIG. 7. In some preferred embodiments the polymorphism is at least one of A959C, A162T, A751T, PI3-1(novel), PI3-2(rs60717610), PI3-3(rs13044826), PI3-4(rs56191952), PI3-5(rs55767422), PI3-6(rs56387543), PI3-7(rs35476703), PI3-8(rs35632684), PI3-9(rs62208416), PI3-10(rs41282752), PI3-11(rs17333103), PI3-12(rs17333180), PI3-13(rs1983649), PI3-14(rs6032040), PI3-15(rs2664581), PI3-16(rs34885285), PI3-17(rs17424474), PI3-18(rs17333381), PI3-19(novel), PI3-20(rs34412950), PI3-21(rs35869085), PI3-22(novel), PI3-23(rs45461302), PI3-24(rs2267864) and/or any combination thereof. In some preferred embodiments at least 2, at least 3, at least 3, at least 5, at least 6, or at least 7 polymorphisms are selected.

In another embodiment, the invention provides a method for determining the predisposition of a subject to develop ARDS the method comprising determining whether the genome of the subject comprises a first polymorphism which is in linkage disequilibrium (LD) with a second polymorphism chosen from the group comprising or consisting of A959C, A162T, and A751T; wherein the presence of the first polymorphism indicates that the subject has a predisposition to develop ARDS. In some preferred embodiments the second polymorphism indicates that the subject has a predisposition to ARDS. In some preferred embodiments the second polymorphism is A959C. In other preferred embodiments the second polymorphism is A162T. In many preferred embodiments the first polymorphism is in the elafin gene. In other preferred embodiments, the first polymorphism is in another gene. In some embodiments the first polymorphism is located within one of the two LD blocks that were constructed with SNPs rs60717610-rs1983649 and rs2664581-rs2267864. In some embodiments the first polymorphism is a polymorphism identified in Table 12 or FIG. 7. In some preferred embodiments the polymorphism is at least polymorphism from the group comprising or consisting of A959C, A162T, A751T, PI3-1, PI3-2(rs60717610), PI3-3(rs13044826), PI3-4(rs56191952), PI3-5(rs55767422), PI3-6(rs56387543), PI3-7(rs35476703), PI3-8(rs35632684), PI3-9(rs62208416), PI3-10(rs41282752), PI3-11(rs17333103), PI3-12(rs17333180), PI3-13(rs1983649), PI3-14(rs6032040), PI3-15(rs2664581), PI3-16(rs34885285), PI3-17(rs17424474), PI3-18(rs17333381), PI3-19, PI3-20(rs34412950), PI3-21(rs35869085), PI3-22, PI3-23(rs45461302), PI3-24(rs2267864) and any combination thereof. In some preferred embodiments at least 2, at least 3, at least 3, at least 5, at least 6, or at least 7 polymorphisms are selected.

In another embodiment the invention provides a method for determining the predisposition of a subject to develop ARDS the method comprising determining whether the genome of the subject comprises a first polymorphism which is in linkage disequilibrium (LD) with a second polymorphism in the elafin gene, wherein the presence of the first polymorphism indicates that the subject has a predisposition to develop ARDS.

In another embodiment the invention provides a method of predicting whether a subject has a predisposition to develop Acute Respiratory Distress Syndrome (ARDS) the method comprising determining whether the genome of the subject comprises the variant allele of A959C; wherein the presence of the allele indicates that the subject has a predisposition to developing ARDS. In some preferred embodiments the subject has extrapulmonary injury.

In a related embodiment the invention provides a method of predicting whether a subject has a predisposition to develop Acute Respiratory Distress Syndrome (ARDS) the method comprising determining whether the genome of the subject comprises haplotype Hap2 (TTC); wherein the presence of the haplotype indicates that the subject has a predisposition, to develop ARDS. In some preferred embodiments the subject has extrapulmonary injury.

Accordingly, in related embodiments combinations of the provided methods may be used. For example, any of the previously recited methods for identifying a subject that is at high risk of developing ARDS (e.g., determining the ratio between elafin and HNE in a test sample from the subject or determining the amount of elafin present in a test sample from the subject) may further comprise determining whether the genome of the subject comprises at least one single nucleotide polymorphism (SNP) known to be associated with an increased risk of ARDS. For example one possible combination provides a method of identifying a subject that is at high risk of developing ARDS by determining the ratio between elafin and HNE in a test sample from the subject; and comparing the foregoing ratio to the ration between elafin and HNE in a control sample, wherein a decrease in the elafin/HNE ration indicates that the subject is at high risk of developing ARDS; said method further comprising determining whether the genome of the subject comprises at least one single nucleotide polymorphism (SNP) known to be associated with an increased risk of ARDS. In some embodiments the polymorphism may be A959C, A162T, or A751T, a polymorphisms identified in Table 12 or FIG. 7, and/or any combination thereof.

In another embodiment the invention provides a method of predicting whether a subject is at high risk of developing Acute Respiratory Distress Syndrome (ARDS) the method comprising determining the amount of elafin present in a test sample from the subject; and comparing the amount of elafin in the test sample to the amount of elafin present in a control sample, determining the ratio of elafin:neutrophil elastase present in a test sample from the subject; and comparing the ratio of elafin:neutrophil elastase in the test sample to the ratio of elafin:neutrophil elastase present in a control sample wherein an increased amount of elafin in the test sample relative to the amount of elafin in the control sample and/or an decreased ratio of elafin:neutrophil elastase in the test sample relative to the ratio of elafin:neutrophil elastase in the control sample (or alternatively and increased ratio of neutrophil elastase:elafin relative to the ratio of elafin:neutrophil elastase in the control sample) indicates that the subject is at high risk of developing ARDS indicates that the subject is at high risk of developing ARDS.

One of skill in the art will appreciate that various other combinations of the provided methods may be employed to predicting whether a subject is at high risk of developing ARDS: e.g., determining the ratio of neutrophil elastase:elafin present in a test sample from the subject wherein an increased ratio of neutrophil elastase:elafin in the test sample relative to the ratio of neutrophil elastase:elafin in the control sample indicates that the subject is at high risk of developing ARDS, said method further comprising one or more of the following: determining whether the genome of the subject comprises haplotype Hap2 (TTC); determining whether the genome of the subject comprises a first polymorphism which is in linkage disequilibrium (LD) with a second polymorphism chosen from the group comprising or consisting of A959C, A162T, A751T, and any combination thereof; determining in the subject one or more of red blood cell count, blood gas measurement; blood pressure measurement, measurement of abnormal breathing sounds by chest auscultation, measurement of effusion and diffuse shadowing on chest radiographs, assessment of cyanosis of the lips or nailbeds, or measurement of breathing frequency.

In a related aspect, the invention further provides kits for use with the methods of the invention. The kits may contain a reagent to determine the amount of elafin and/or HNE in a subject sample; instructions for use; and, optionally, a reagent for isolating a sample from the subject. A kit may also comprise an oligonucleotide that specifically detects a polymorphism in or near the elafin gene. For example, a kit may include oligonucleotides that specifically detect A959C, A162T, A751T and/or any of the SNPs described herein.

In another aspect, the invention also provides methods of treating Acute Respiratory Distress Syndrome in a subject. The treatment methods include administering to a subject diagnosed with ARDS an effective amount of elafin such that the ARDS is treated. In certain embodiments the elafin is administered to a subject in combination with traditional treatment regimes for ARDS.

### BRIEF DESCRIPTION OF THE FIGURES

FIG. 1. Comparison of the expression fold-changes of selected genes measured by microarray and quantitative RT-PCR. Of 11 pairs of RNA samples used in microarray analysis, only 6 pairs had enough RNA for quantitative RT-PCR analysis, including 5 ARDS cases and one control. Gene PI3, IL8, and HPGD were chosen as having large differential expression between acute stage and stable stage of ARDS, as well as being identified repeatedly in Medline literature mining. Gene CYP4F3 with marginal expression change and gene MMP9 with no change were chosen as reference genes in validation. The results of gene ACTB was also illustrated, which was originally selected as endogenous control. The y-axis displays the fold change of the recovery-stage to the acute-stage of ARDS.

FIG. 2. Levels of pre-elafin (PI3) in plasma of ARDS subjects and critical ill patients who did not develop ARDS (controls). Each ARDS case provides on one plasma sample. Based the date of sample collection relative to the ARDS diagnosis date, including pre-diagnosis group (Day −5 to Day −1), day of diagnosis group (Day 0), and post-diagnosis group (Day 1 to Day 3). Sample 1 of control was collected during the first two days of ICU admission. There was no statistically significant difference in baseline characteristics between ARDS cases and controls, except that ARDS cases were more frequently received transfusion (p=0.009). Significant results of t-test: ARDS pre-diagnosis vs. post diagnosis, p=0.0009; ARDS day of diagnosis vs. post diagnosis, p=0.05; ARDS pre-diagnosis vs. control, p=0.01.

FIG. 3. Levels of MMP9 in plasma of ARDS subjects and critical ill patients who did not develop ARDS (controls). Each ARDS case provides on one plasma sample. Based the date of sample collection relative to the ARDS diagnosis date, including pre-diagnosis group (Day −5 to Day −1), day of diagnosis group (Day 0), and post-diagnosis group (Day 1 to Day 3). Sample 1 of control was collected during the first two days of ICU admission. There was no statistically significant difference in baseline characteristics between ARDS cases and controls, except that ARDS cases were more frequently received transfusion (p=0.009). There is no statistically significant difference existed in plasma levels of MMP-9 between ARDS cases and at-risk controls.

FIG. 4. Levels of pre-elafin (PI3) in plasma of critical ill subjects who did not develop ARDS (controls). All controls provided paired plasma samples. Sample 1 was collected during the first two days of ICU admission; Sample 2 was collected three days later after Sample 1. Significant increase of plasma PI3 levels (mean ratio=1.53, 95% CI, 1.28-1.78; p=0.0007) in Sample 2 (three days after the first 48 hours of ICU admission) was observed using paired t-test.

FIG. 5. Box plot of plasma profiles of PI3, SLPI, HNE, and HNE/PI3 among pre-ARDS, the ARDS and the controls. Pre-ARDS: plasma sample (n=19) collected during 7-day period before ARDS diagnosis; ARDS: samples (n=67) collected within 48-hour of ARDS diagnosis; Control: samples (n=63) collected within 48-hour of ICU admission; Ref: anonymous plasma samples from healthy individual (n=28). Pairwise comparison:

FIG. 6. Box plot of plasma profile changes in clinical progress of ARDS. Pre-ARDS: plasma sample (n=19) collected during 7-day period before ARDS diagnosis; ARDS: samples (n=67) collected within 48-hour of ARDS diagnosis; Post-ARDS: samples (n=) collected between Day 2 and Day 4 of ARDS diagnosis.

FIG. 7 (A) Location of the SNPs identified in PI3 gene. The first nucleotide of the translation start site is denoted as +1. Dark shaded and light shaded boxes represent exons and introns respectively. Horizontal bars indicate the transcription factor predicted to bind to one of the alleles of rs56191952, rs55767422, rs56387543, rs35476703 and rs62208416, respectively (20). FIG. 7 (B) Linkage disequilibrium plot for SNPs in the PI3 gene. The value within each diamond is D′ between pairs of SNPs expressed in percentages. The black-to-white gradient reflects higher to lower linkage disequilibrium values (black, high; white low). Unreported values reflects D′=1 (100%)

FIG. 8 Effects of PI3 rs2664581 polymorphism on plasma PI3 level. The ARDS samples were collected within 48-hour of ARDS diagnosis, and the control samples were collected within 48-hour of ICU admission. Geometric mean of plasma PI3 is shown in columns with error bar representing 95% confidence interval. The subgroup of the ARDS with AA genotype (n=42) had significant lower plasma PI3 than the ARDS with AC/CC genotypes (n=21, P=0.021) and the control with AA genotype (n=40, P=0.043) in crude analysis. After adjustment for age, gender, type of lung injury, pre-admission steroid use, septic shock, and APACHE III score, these differences remained significant (P=0.009 and 0.007, respectively).

FIG. 9. Flow diagram of study design and patient selection for case-control study.

FIG. 10. PI3 peptide sequence comparison among different species. PI3 peptide sequences from different species were retrieved from National Center for Biotechnology Information (NCBI) database using BLAST search engine. Sequence alignment was conducted on Jalview software (35). Arrow indicates the position of non-synonymous polymorphism (T34P, rs2664581). Black underlines indicate the positions of 5 transglutaminase substrate motifs.

FIG. 11. Haploview LD (D′) of SNPs of PI3 gene constructed using genetic data derived from HapMap. The D′ for any two SNPs is presented in the box representing their intersection. The red boxes represent the strongest LD, the gray ones represent lower linkage disequilibrium values.*Tagging SNPs predicted by the HapMap CEU sample.

### DETAILED DESCRIPTION OF THE INVENTION

The present invention is based, at least in part, on the discovery that prior to the onset of ARDS (e.g., the onset of the acute phase of ARDS, e.g., prior to clinical diagnosis), the amount of elafin is significantly increased in a biological sample derived from a subject at risk of developing ARDS and who subsequently develops or is diagnosed with ARDS, as compared to the amount of elafin in a biological sample from a control subject a subject at risk of developing ARDS who does not develop ARDS and/or has no prior history of ARDS. Furthermore, the present invention is based, at least in part, on the discovery that the amount of elafin is significantly decreased in a biological sample derived from a subject that is in the recovery phase of ARDS, e.g., has received an efficacious treatment, as compared to the amount of elafin in a biological sample from a subject, e.g., in the acute phase of ARDS. In addition, the present invention is based, at least in part, on the discovery that the ration of elafin/HNE is significantly lower in samples with the onset of ARDS as compared to at risk controls.

Accordingly, the present invention provides methods for identifying subjects or patients are at high risk of developing Acute Respiratory Distress Syndrome (ARDS) by determining the amount of elafin present in a subject sample, by determining the ratio of elafin/HNE in a subject sample, methods for monitoring the efficacy of a treatment regimen for ARDS as well as methods of treatment for ARDS.

In additional aspects, the present invention is based, at least in part, on the discovery that certain nucleotide polymorphisms are associated with an increased risk for developing ARDS in a subject.

Various aspects of the invention are described in further detail in the following subsections:

### I. Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Acute Respiratory Distress Syndrome” or “ARDS” is the presence of pulmonary edema in the absence of volume overload or depressed left ventricular function. One of ordinary skill in the art can readily diagnose ARDS in a subject based on, for example, the American-European Consensus Conference Committee (AECCC) criteria which takes into account the ratio of partial pressure of arterial oxygen to the fraction of inspired oxygen (among other indicators) as a means to categorize lung injury. (See, e.g., G Bernard et al. The Consensus Committee. Intensive care medicine 20 (3), 225-32 1994), the contents of which are incorporated herein by reference). In brief, the AECCC criteria for ARDS are: acute onset, bilateral infiltrates on a chest radiograph, pulmonary-artery wedge pressure ≦18 mm of mercury (as measured, e.g., by a pulmonary artery catheter) (alternatively, the absence of clinical evidence of left atrial hypertension), and a measured ratio of the partial pressure of arterial oxygen to the fraction of inspired oxygen of ≦200.

ARDS typically has two phases; the first is the “acute phase”, or “exudative phase” which manifests itself with the onset of respiratory failure often characterized by arterial hypoxia. For many subjects this progresses to an even more severe phase of fibrosing alveolitis. In this case, hypoxia is persistent, pulmonary compliance continues to degrade, there may be evidence of increased fibrosis in the lung, and, eventually, death may occur. Other subjects enter the “recovery phase” wherein hypoxia begins to reverse and pulmonary function may, in some cases, return to normal.

A high concentration of neutrophil elastase (HNE) is stored in azurophil granules of neutrophils. Upon activation, however, HNE can be released rapidly into the extracellular space and cause tissue damage (Kawabata, K., et al. Eur J Pharmacol. 2002. 451:1-10.). Proteinase inhibitors are imported to protect tissues from unregulated proteolysis, whereas low molecular weight inhibitors are especially critical in local protection by their capability of penetrating into the “microenvironment” created by neutrophil to sequester HNE (Rice, W. G., and Weiss, S. J. 1990. Science 249:178-181.). A local imbalance between proteinases and inhibitors results in pulmonary parenchyma damage by leakage of a protein-rich fluid into the interstitium and alveolar spaces, which is the major mechanism for activated neutrophils initiating and propagating ARDS (Weiland, J. E., et al. Am Rev Respir Dis. 1986 133:218-225.).

A subject that is at risk of developing ARDS includes any subject having a condition or disease that places the subject at higher than normal risk of developing an inflammatory response that may lead to lung dysfunction. This includes any subject or patient that has sepsis, pneumonia, severe burns, bacterial infection, pancreatitis, or viral infection (e.g., SARS, H5N1, influenza virus, coronavirus, etc.). Also included are subjects which have experienced direct lung injury via a direct blow to the chest or other lung trauma due to, but not limited to, toxin inhalation (including chemical fumes, acids, etc.), smoke inhalation, and/or inhalation of abrasive particulates. Also included in at-risk subjects are those experiencing any inflammatory response which may affect the lungs, subjects who have had recent massive transfusions and subjects with congestive heart failure.

“Elafin”, also referred to as peptidase inhibitor 3 (PI3) and Trappin 2, is a protease inhibitor that functions in both normal homeostasis and at sites of inflammation. The functions of elafin include, for example, antiprotease and antimicrobial activity as well as modulation of the response to LPS (lipopolysaccharide) stimulation. Elafin is produced as a 117 amino acid (˜12.3 kDa) protein referred to as “pre-elafin” (Schalkwijk J, et al. Biochem J. 1999. Jun. 15; 340 (Pt 3):569-77.). This form is fully functional and contains a 22 amino acid signal peptide at the N-terminus, an N-terminal ‘cementoin’ domain which facilitates transglutaminase-mediated cross-linkage on to polymers or ECM components and a globular C-terminus, containing the proteinase inhibitor moiety. Cleavage of the signal protein yields a mature protein of about 9.9 kDa which is also fully functional. Unless specifically indicated, the term “elafin” is used interchangeably with and is equivalent to the term “pre-elafin”. The nucleotide and amino acid sequence of elafin and preelafin are known and can be found in, for example, GenBank Accession No. gi:31657130. In contrast to SLPI, elafin has a narrow spectrum of inhibition specifically toward HNE and proteinases 3 (Moreau, et al. Biochimie. 2008. 90:284-295.). Moreover, elafin has a unique N-terminal domain containing several transglutaminase substrate motifs that can nail the molecule to extracellular matrix (ECM) proteins via covalent cross-linking and still inhibit HNE (Nara, K., et al. J Biochem (Tokyo). 1994. 115:441-448., Guyot, N., et al. Biochemistry. 2005. 44:15610-15618). Thus, elafin can provide more efficient local protection in lung. In addition to its role as protease inhibitor, PI3 has also been demonstrated to have many biological functions including antimicrobial, anti-inflammatory and anti-viral activities and immuno-modulatory functions (Moreau, T., et al. 2008. Biochimie, 90, 284-295; Williams, S. E., et al. 2006. Clin. Sci. Lond., 110, 21-35.).

“Human neutrophil elastase”, (HNE) is an acid-independent, inflammatory serine protease predominantly expressed by neutrophils. This enzyme hydrolyzes proteins within specialized neutrophil lysosomes, called azurophil granules, as well as proteins of the extracellular matrix following the protein's release from activated neutrophils. HNE is produced as a 267 pre-protein and contains a 29 amino acid signal peptide. The mature form of HNE contains amino acids 30-267 of the pre-protein form. The nucleotide and amino acid sequences of HNE are known and can be found in, for example, GenBank Accession No. gi:4503549 (NP 001963; NM 001972).

As described herein, the amount of elafin, e.g., the amount of mRNA and/or protein, is increased in a biological sample from a subject at high risk of developing ARDS as compared to the amount of elafin, e.g., the amount of mRNA and/or protein, in a control sample. As also described herein, the amount of elafin, e.g., the amount of mRNA and/or protein, is decreased in a biological sample from a subject that has received an efficacious treatment for ARDS as compared to the amount of elafin, e.g., the amount of mRNA and/or protein, in a biological sample derived from the subject prior to the initiation of treatment.

As used herein, the term “level” or “amount”, with respect to elafin and/or HNE, refers to the mRNA level, and/or the protein level of elafin and/or HNE in a cell or sample. The amount may be either (a) an absolute amount as measured in molecules, moles or weight per unit volume or cells; (b) a relative amount, e.g., measured by densitometric analysis; or (c) the level of activity of the elafin protein (e.g., antiprotease activity) and/or HNE activity (e.g., protease activity).

The term “altered amount” refers to increased or decreased level of elafin protein, activity or mRNA in a biological sample of a subject, as compared to the level of the elafin in a control sample. The amount of elafin, in a cell or a sample derived from a subject is “altered” (“increased or decreased” or “higher or lower”) than the control amount of elafin if the difference in the amount of elafin in the test sample compared to the amount of elafin in the control sample is greater than the standard error of the assay employed to assess the amount. The amount of elafin, in a cell or a sample derived from a subject can be considered “higher” or “lower” than the control amount, if the difference in the control amount and the sample amount is at least about two, and preferably at least about three, four, or five times, higher or lower, respectively, than the standard error of control and sample measurements of elafin.

The term “control amount” of elafin, as used herein, refers to the amount of elafin in a cell or a sample derived from a subject that healthy or, preferably, a subject that was at risk for ARDS but which did not develop the disease. In general, the “control amount” may, for example, be determined by calculating the average amount of elafin present in samples that are known to contain elafin. In specific instances of the invention, it may be advantageous to compare a sample from a test subject to a sample from a healthy subject not afflicted with ARDS. Furthermore, in some embodiments, the amount of elafin in a subject will be monitored during the course of disease. In that case, multiple samples will be taken from the test subject at different time points.

The term “polymorphism” refers to the coexistence of more than one form of a gene, or portion thereof, or a segment of DNA. A portion of a gene or segment of DNA of which there are at least two different forms, i.e., two different nucleotide sequences, is referred to as a “polymorphic region.” A polymorphic locus can be a single nucleotide, the identity of which differs in the other alleles. A polymorphic locus can also be more than one nucleotide long. The allelic form occurring most frequently in a selected population is often referred to as the reference and/or wildtype form. Other allelic forms are typically designated or alternative or variant alleles. Diploid organisms may be homozygous or heterozygous for allelic forms. A diallelic or biallelic polymorphism has two forms. A trialleleic polymorphism has three forms.

The term “single nucleotide polymorphism” (SNP) refers to a polymorphic site occupied by a single nucleotide, which is the site of variation between allelic sequences. A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site.

SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect.

The term “linkage” describes the tendency of genes, alleles, loci or genetic markers to be inherited together as a result of their location on the same chromosome. It can be measured by percent recombination between the two genes, alleles, loci, or genetic markers. The term “linkage disequilibrium,” also referred to herein as “LD,” refers to a greater than random association between specific alleles at two marker loci within a particular population. In general, linkage disequilibrium decreases with an increase in physical distance. If linkage disequilibrium exists between two markers, or SNPs, then the genotypic information at one marker, or SNP, can be used to make probabilistic predictions about the genotype of the second marker.

As used herein, the term “detect” with respect to polymorphic elements includes various methods of analyzing for a polymorphism at a particular site in the genome. The term “detect” includes both “direct detection,” such as sequencing, and “indirect detection,” using methods such as amplification and/or hybridization.

The term “probe” refers to any molecule that is capable of selectively binding to elafin, for example, an elafin nucleotide transcript or elafin protein. Probes can be synthesized by one of skill in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic monomers.

As used herein, “antibody” includes, by way of example, naturally-occurring forms of antibodies (e.g., IgG, IgA, IgM, IgE) such as polyclonal antibodies and monoclonal antibodies, and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

As used herein, a “subject” is any animal, such as a mammal, and includes, without limitation, humans, mice, monkeys, dogs, cats, mice, rats cows, horses, goats, sheep as well as other farm and pet animals.

ARDS is “treated” if at least one symptom of ARDS is alleviated, terminated, slowed, or prevented. As used herein, ARDS is also “treated” if a subject enters recovery phase or if a subject is protected from a future case of ARDS.

As used herein, the “traditional clinical measurements” or the “traditional clinical tests” which may be combined with the methods of the present invention to diagnose or monitor ARDS are chest radiographs, red blood cell count, blood gas measurements (for example, the ratio of partial pressure of arterial oxygen to the fraction of inspired oxygen), blood pressure, chest auscultation, and observations of the subject including observations of cyanosis of the lips or nailbeds, and rapid or labored breathing (e.g., measuring the breathing frequency of subject as compared to controls). Chest radiographs may be X-rays, CT scans, PET scans, or other methods known in the art. ARDS subjects often display dense infiltrates or effusions in the lung which can be seen visually or assessed by measuring the diffuse shadowing in a chest radiograph. Furthermore cyanosis of the lips or nailbeds may be observed by a clinician, assessed by measurement of lip/nailbed discoloration, or assessed by measurement of blood flow to the lips or nailbeds (e.g., by a duplex/doppler ultrasound). A reduced blood flow as compared to healthy or control subjects is supportive of a diagnosis of ARDS. Chest auscultation, as performed by one of skill in the art, will often reveal abnormal breathing sounds such as crackles that suggest fluid build up in the lungs whfch may be associated with ARDS. Additionally, the traditional clinical measurements or tests may encompass any tests, measurements, or observations which indicate the onset of ARDS or alterations in subject status. These tests and clinical observations are well known in the art, and may be performed by any method acceptable to a skilled medical practitioner.

A “kit” is any manufacture (e.g. a package or container) comprising at least one reagent, e.g. a probe, for specifically detecting elafin, the manufacture being promoted, distributed, or sold as a unit for performing the methods of the present invention.

### II. Prognostic and Diagnostic Methods of the Invention

The present invention provides methods of identifying subjects who are at “high risk” of developing ARDS (e.g., will more likely than not develop ARDS). A skilled artisan will appreciate that subjects which have a “high risk” as described herein, have an increased risk of developing ARDS as compared to other subjects which are at risk of developing the disease merely because they have a condition or disease that places the subject at higher than normal risk of developing an inflammatory response that may lead to lung dysfunction.

In some embodiments, these methods comprise determining the amount of elafin, e.g., the amount of elafin mRNA and/or protein, in a biological sample from the subject and comparing the foregoing amount with a control amount of elafin present in a control sample, e.g., a sample from a subject at risk of developing ARDS, but who does not develop ARDS (and who has never had ARDS), or from a healthy subject. An increased amount of elafin present in the subject sample as compared to the amount of elafin present in the control sample indicates that the subject will develop ARDS. In addition, a lower or about equal amount of elafin in the subject sample as compared to the control sample indicates that the subject is unlikely to develop ARDS.

The present invention also provides methods of identifying subjects who are at high risk of developing ARDS by determining the ratio of elafin/neutrophil elastase mRNA and/or protein in a biological sample from the subject and comparing the foregoing ration with a control ration of elafin/neutrophil elastase in a control sample. A decreased ratio of elafin/neutrophil elastase present in the subject sample as compared to the ratio in the control sample is indicative that the subject is at high risk of developing ARDS, and a significant increase in the ratio is indicative that the subject is unlikely to develop ARDS. Alternatively, an increased ratio of HNE/elafin present in the subject sample as compared to the ratio in the control sample is indicative that the subject is at high risk of developing ARDS, and a significant increase in the ratio is indicative that the subject is unlikely to develop ARDS. In some embodiments the subject has direct lung injury.

In other embodiments the invention provides methods to determine the predisposition of a subject to develop ARDS. A subject with a predisposition to develop ARDS may be considered to have a “high risk” of developing the disease. A subject with a predisposition to develop ARDS includes subjects which are genetically predisposed to the disease by, e.g., carrying a SNP which is correlated with a higher incidence of ARDS. Detecting a SNP which is correlated with ARDS in the genome of a subject indicates that the subject is predisposed to develop the disease.

The present invention also provides methods for monitoring the efficacy of a treatment regimen for a subject with ARDS. These methods generally involve determining the amount, e.g., mRNA and/or protein in a pair of samples (a first sample not subjected to the treatment regimen and a second sample subjected to at least a portion of the treatment regimen) is assessed. In certain embodiments, a lower amount of elafin in the second sample, relative to the first, is an indication that the treatment regimen is efficacious for treating ARDS. In addition, a higher or about equal amount of elafin in the second sample, relative to the first, is an indication that the treatment regimen is not efficacious for treating ARDS. In other embodiments, a higher ratio of elafin/HNE in the second sample, relative to the first, is an indication that the treatment regiment is efficacious for treating ARDS, whereas a lower ratio of elafin/HNE in the second sample is an indication that the treatment regimen is not efficacious for treating ARDS.

According to the methods of the invention, elafin levels, HNE levels, and/or the ratio of elafin/HNE (or the inverse ratio of HNE/elafin) may be used to monitor the clinical progress of ARDS. For example, in some embodiments levels of HNE which are higher than those in control subjects indicates progression of the disease or lack of efficacy of treatment. In other embodiments a reduced amount of HNE as compared to the level of HNE in a previous measurement in the same subject indicates that a treatment is efficacious. One of skill in the art would understand that various combinations of measurements of HNE, elafin and/or the ratio of elafin/HNE as compared to measurements in control subjects or to measurements at previous time points may be used to monitor the clinical progression of ARDS. In some embodiments, the subject is at risk of developing ARDS but has not been diagnosed with ARDS (pre-diagnosis subject). In other embodiments the subject has been diagnosed with ARDS, and the methods described herein may be use to monitor clinical progression and/or treatment.

Samples useful in the methods of the invention include any tissue, cell, biopsy, or bodily fluid sample that contains elafin and/or HNE protein or mRNA including, but not limited to, whole blood, serum, plasma, buccal scrape, hair follicle, skin cells, saliva, mucus, lymph, cerebrospinal fluid, urine, stool, or bronchioalveolar lavage. In one embodiment, the biological sample is a whole blood sample. In another embodiment, the sample is a plasma sample. In another embodiment, the biological sample is a lung tissue sample. In yet another embodiment, the biological sample is a bronchioalveolar lavage sample (e.g., a nonbronchoscopic bronchioalveolar lavage sample).

Samples may be obtained from a subject by a variety of techniques known in the art including, for example, by the use of a biopsy or by scraping or swabbing an area or by using a needle to aspirate bodily fluids. Methods for collecting various samples are well known in the art. In one embodiment, a nonbronchoscopic bronchioalveolar lavage sample is obtained (e.g., Fujitani, S. and V. L. Yu, J Intensive Care Med, 2006. 21(1): p. 17-21).

Once obtained, samples suitable for quantitating the amount of elafin (e.g., mRNA, protein), may be fresh, frozen, or fixed according to methods known to one of skill in the art. Suitable tissue samples may be solubilized and/or homogenized and subsequently analyzed as soluble extracts.

Once the sample is obtained any method known in the art to be suitable for quantitating the amount of elafin and/or HNE may be used (either at the nucleic acid or at the protein level). Such methods are well known in the art and include, for example, but are not limited to Western blots, Northern blots, Southern blots, immunohistochemistry, ELISA, e.g., amplified ELISA, immunoprecipitation, immunofluorescence, flow cytometry, immunocytochemistry, mass spectrometrometric analyses, e.g., MALDI-TOF and SELDI-TOF, nucleic acid hybridization techniques, nucleic acid reverse transcription methods, and nucleic acid amplification methods.

In general, according to the methods of the invention, the difference in the amount of elafin and/or the elafin/HNE ratio in the test sample(s) as compared to the control sample which is predictive of the subject developing ARDS is at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater than the standard error of the assessment method.

The methods of the present invention can be practiced in conjunction with any other method used by the skilled practitioner to diagnose, treat, and/or monitor the progression of ARDS. For example, the methods of the invention may be performed in conjunction with traditional clinical measurements including, but not limited to, chest radiographs, red blood cell count, blood gas measurements, blood pressure measurements, chest auscultation, and other relevant observations of the subject which would be obvious to one of skill in the art (including observations of cyanosis of the lips or nailbeds, and rapid or labored breathing). One of skill in the art will appreciate that the methods described herein may be used in combination. For example, a method for determining the predisposition of a subject to develop ARDS comprising determining whether the genome of the subject comprises at least one single nucleotide polymorphism (SNP) known to be associated with an increased risk of ARDS, to thereby determine the predisposition of the subject to develop ARDS, may be combined with a method of predicting whether a subject is at high risk of developing ARDS the method comprising: determining the amount of elafin present in a test sample from the subject; and comparing the amount of elafin in the test sample to the amount of elafin present in a control sample, wherein an increased amount of elafin in the test sample relative to the amount of elafin in the control sample indicates that the subject is at high risk of developing ARDS. Such combinations may provide reinforcing evidence that a subject is predisposed or at high risk of developing ARDS (e.g., combining methods for determining plasma elafin levels and determining the elafin/HNE ratio or the level of HNE) or the combinations may be used determine whether a subject is at high risk of developing ARDS and then to monitor the subject's clinical progression (detection of a particular SNP followed by monitoring of a subject's plasma elafin level, HNE level, or elafin/HNE ratio). In a further embodiment the combinations may be used determine whether a subject is at high risk of developing ARDS (e.g., by determining whether the genome comprises a particular SNP, determining the elafin level, HNE level, or ratio of elafin/HNE in a subject and comparing the measurement to a control sample) and then to monitor the efficacy of treatment of the disease (e.g., by monitoring the plasma elafin level, HNE level, or elafin/HNE ratio in the subject at certain time intervals and comparing the measurements).

A. Protein Assays

In various embodiments of the methods of the invention, the amount elafin and/or HNE is determined by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In certain embodiments, the agent for detecting an elafin polypeptide is an antibody capable of binding to an elafin polypeptide. In other embodiments, the agent for detecting an HNE polypeptide is an antibody capable of binding to an HNE polypeptide. Antibodies can be polyclonal, or more preferably, monoclonal. An intact antibody, or a fragment thereof (e.g., Fab or F(ab′)2) can be used. Antibodies for detecting elafin polypeptide and/or HNE polypeptide are well known in the art and are available commercially from, for example, Cell Sciences, Abcam, ABR-Affinity, BioReagents, GeneTex, Hycult Biotechnology, Novus Biologicals, R&D Systems, or Santa Cruz Biotechnology.

In related embodiments, the antibody is bound to a detectable label. For example, the antibody can be coupled to a radioactive isotope such as: ³H, ¹³¹I, ³⁵S, ¹⁴C, and preferably ¹²⁵I. Alternatively, the antibody may be labeled with a fluorescent compound such as fluorescein isothiocyanate, rhodamine, phycoerytherin, phycocyanin, allophycocyanin, o-phthaldehyde and fluorescamine; a fluorescence emitting metal such as ¹⁵²Eu, or others of the lanthanide series; a chemiluminescent compound such as luminol, luciferin, isoluminol, theromatic acridinium ester, imidazole, acridinium salt and oxalate ester; a bioluminescent compound including luciferin, luciferase and acquorin.

Antibodies can also be detectably labeled is by linking the same to an enzyme. This enzyme, in turn, when later exposed to its substrate, will react with the substrate in such a manner as to produce a chemical moiety which can be detected, for example, by spectrophotometric, fluorometric or by visual means. Enzymes which can be used to detectably label the antibody include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

The antibodies are used to detect and/or quantify the elafin polypeptide and/or HNE polypeptide using any of a number of well recognized immunological binding assays which the skilled artisan can readily adapt for use in determining the amount of elafin protein (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Antibodies, or antibody fragments, which specifically bind to elafin or the HNE polypeptide can be used in methods such as Western blots or other electrophoretic techniques well known to those of skill in the art to detect elafin protein (see generally, R. Scopes (1982) Protein Purification, Springer-Verlag, N.Y.; Deutscher, (1990) Methods in Enzymology Vol. 182: Guide to Protein Purification, Academic Press, Inc., N.Y.). In one embodiment, the amount of elafin is determined by Wester blot analysis and subsequent densitometric analysis.

Radioimmunoassays are also useful in the methods of the invention. The concentration of antigen (i.e., elafin, HNE) in a sample (i.e. biological sample) is measured by having the antigen in the sample compete with a labeled (i.e. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (i.e. covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

Immunoradiometric assays (IRMA), in which the antibody reagent is radioactively labeled, may also be used to determine the level of elafin. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

Other types of immunoassays which may be used include “Enzyme-Linked Immunosorbent Assay (ELISA)”, which is a technique for detecting and measuring the concentration of an antigen using a labeled (i.e. enzyme linked) form of the antibody. In a “sandwich ELISA”, an antibody (i.e. to BMDC) is linked to a solid phase (i.e. a microtiter plate) and exposed to a biological sample containing antigen (i.e. BMDC). The solid phase is then washed to remove unbound antigen. A labeled (i.e. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and β-galactosidase. The enzyme linked antibody reacts with a substrate to generate a colored reaction product that can be assayed for. In a “competitive ELISA”, antibody is incubated with a sample containing antigen (i.e. BMDC). The antigen-antibody mixture is then contacted with an antigen-coated solid phase (i.e. a microtiter plate). The more antigen present in the sample, the less free antibody that will be available to bind to the solid phase. A labeled (i.e. enzyme linked) secondary antibody is then added to the solid phase to determine the amount of primary antibody bound to the solid phase.

Antibodies or fragments that specifically bind to elafin or HNE may also be used in in vivo techniques in accordance with the methods of the invention. Such methods include introducing into a subject a labeled (e.g., radioactive) antibody directed against elafin which can be detected by standard imaging techniques.

In other embodiments of the invention, proteomic methods, e.g., mass spectrometry, are used for detecting and quantitating elafin and/or HNE. For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis Markers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to detect and quantitate elafin protein. Mass spectrometric methods are described in, for example, U.S. Pat. Nos. 5,622,824, 5,605,798 and 5,547,835, the entire contents of each of which are incorporated herein by reference.

In other embodiments, the amount of elafin protein is assayed by measuring the enzymatic activity of the gene product. Methods of assaying the activity of an elafin are well known to those of skill in the art. For example, the anti-neutrophil elastase activity may be assessed by using a neutrophil elastase-specific substrate N-methoxy-succinyl-Ala-Ala-Pro-Val p-nitroanilide which can be obtained, for example, by Sigma. Neutrophil elastase activity in a sample of interest will liberate p-nitroaniline, which can be using, for example, a spectrophometer at 405 nm over a certain time period. The neutrophil elastase activity measured can then be compared with a sample of known neutrophil elastase activity. Such standards may be designed for a specific experiment or purchased from pharmaceutical vendors, for example, Elastin Products. The difference in the measured activity of neutrophil elastase in the sample of interest and the known sample will indicate the activity level of elafin.

Alternative methods exist for specifically measuring activity of elafin. Elafin selectively inhibits human leukocyte elastase, porcine pancreatic elastase, and proteinase 3, but not trypsin, plasmin, chymotrypsin, or cathepsin G (Molhuizen H O F, et al. 1995. Biol Chem Hoppe Seyler. 1995; 376:1-7; Zaidi, SHE. 1999. J Clin Invest. 1999 Apr. 15; 103(8): 1211-1219). Additional methods for determining the activity and/or amount of elafin are described in, for example, O'Blenes, S B. 2000. Circulation. 102:III-289; and, Nadziejko, C., et al. 1995. Am. J. Respir. Crit. Care Med. November. Vol 152, No. 5, 1592-1598 which are incorporated herein by reference. Furthermore, general methods for assaying protein activity may be used, for example, U.S. Pat. Nos. 6,174,693, and 5,585,235 which are incorporated herein by reference.

B. Nucleic Acid Assays

In another embodiments of the invention, the amount of elafin and/or HNE is determined by measuring and quantitating the amount of elafin nucleic acid and/or HNE nucleic acid. Nucleic acid-based techniques for assessing expression are well known in the art and include, for example, determining the amount of elafin or HNE mRNA in a body sample using probes which bind to elafin or HNE mRNA or cDNA.

Many expression detection methods use isolated RNA. Any RNA isolation technique that does not select against the isolation of mRNA can be utilized for the purification of RNA from cells that express elafin (see, e.g., Ausubel et al., ed., (1987-1999) Current Protocols in Molecular Biology (John Wiley & Sons, New York). Additionally, large numbers of tissue samples can readily be processed using techniques well known to those of skill in the art, such as, for example, the single-step RNA isolation process of Chomczynski (1989, U.S. Pat. No. 4,843,155).

Probes or primers useful in the methods of the invention can be prepared by a variety of standard techniques. For example, various methods of chemically synthesizing polydeoxynucleotides are known, including solid-phase synthesis which has been automated in commercially available DNA synthesizers (See, e.g., Itakura et al., U.S. Pat. No. 4,598,049; Caruthers et al., U.S. Pat. No. 4,458,066; and Itakura, U.S. Pat. Nos. 4,401,796 and 4,373,071, incorporated herein by reference).

Methods of detecting and/or quantifying the gene transcript (mRNA or cDNA made there from) using nucleic acid hybridization techniques are known to those of skill in the art (see Sambrook et al. supra). For example, one method for evaluating the presence, absence, or amount of cDNA involves a Southern transfer as described above. Briefly, the mRNA is isolated (e.g. using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such assays involves preparing a sample or reaction mixture that may contain a marker, i.e., elafin, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or amount of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, polyethylene, dextran, amylases, natural and modified celluloses, polyacrylamides, gabbros, and magnetite.

In order to conduct assays with the above-mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example, through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In another embodiment, the amount of mRNA corresponding to elafin or HNE can be determined both by in situ and by in vitro formats in a biological sample using methods known in the art. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated nucleic acid can be used in hybridization or amplification assays that include, but are not limited to, Southern or Northern analyses, polymerase chain reaction analyses and probe arrays. One preferred diagnostic method for the detection of the amount of mRNA involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the amount of mRNA encoded by the markers of the present invention.

The probes can be full length or less than the full length of the nucleic acid sequence encoding the protein. Shorter probes are empirically tested for specificity. Preferably nucleic acid probes are 20 bases or longer in length. (See, e.g., Sambrook et al. for methods of selecting nucleic acid probe sequences for use in nucleic acid hybridization.) Visualization of the hybridized portions allows the qualitative determination of the presence or absence of cDNA.

An alternative method for determining the amount of a transcript corresponding to elafin or HNE in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, elafin expression is assessed by quantitative fluorogenic RT-PCR (i.e., the TaqMan™ System). Such methods typically utilize pairs of oligonucleotide primers that are specific for the marker. Methods for designing oligonucleotide primers specific for a known sequence are well known in the art.

Fluorogenic rtPCR may also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

In another embodiment, the amount of elafin and/or HNE is assessed by preparing genomic DNA or mRNA/cDNA (i.e. a transcribed polynucleotide) from cells in a subject sample, and by hybridizing the genomic DNA or mRNA/cDNA with a reference polynucleotide which is a complement of a polynucleotide comprising the marker, and fragments thereof. cDNA can, optionally, be amplified using any of a variety of polymerase chain reaction methods prior to hybridization with the reference polynucleotide. Expression amounts of one or more markers can likewise be detected using quantitative PCR (QPCR) to assess the amount of expression of the marker(s). Alternatively, any of the many known methods of detecting mutations or variants (e.g. single nucleotide polymorphisms, deletions, etc.) of a marker of the invention may be used to detect occurrence of a mutated marker in a subject.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of elafin or HNE. If polynucleotides complementary to or homologous with elafin are differentially detectable on the substrate (e.g. detectable using different chromophores or fluorophores, or fixed to different selected positions), then the amounts of expression of a plurality of markers, including elafin, can be assessed simultaneously using a single substrate (e.g. a “gene chip” microarray of polynucleotides fixed at selected positions). When a method of assessing marker expression is used which involves hybridization of one nucleic acid with another, it is preferred that the hybridization be performed under stringent hybridization conditions.

In one embodiment of the invention, microarrays are used to determine the amount of elafin and/or HNE expression. Microarrays are particularly well suited for this purpose because of the reproducibility between different experiments. DNA microarrays provide one method for the simultaneous measurement of the expression amounts of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels (amounts). See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

In another embodiment, a combination of methods to assess the expression of elafin and/or HNE is utilized.

The assays of this invention are scored (as positive or negative or amount of polypeptide and/or mRNA) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

As an alternative to making determinations of the amount of elafin or HNE based on the absolute expression level of the marker, determinations may be based on the normalized expression level of the marker. Expression levels are normalized by correcting the absolute expression level of a marker by comparing its expression to the expression of a protein that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a control sample, a sample from the same subject at different points in time, or between samples from different sources.

Alternatively, the expression level can be provided as a relative expression level. To determine a relative expression level of a marker, the level of expression of the marker is determined for 10 or more samples of normal versus control cell isolates, preferably 50 or more samples, prior to the determination of the expression level for the sample in question. The mean expression level of each of the proteins assayed in the larger number of samples is determined and this is used as a baseline expression level for the marker, i.e., elafin. The expression level of the marker determined for the test sample (absolute level of expression) is then divided by the mean expression value obtained for that marker. This provides a relative expression level.

Preferably, the samples used in the baseline determination will be from samples of the same tissue type. The choice of the cell source is dependent on the use of the relative expression level. Using expression found in normal, control tissues as a mean expression score aids in validating whether the amount of elafin assayed is specific to the tissue from which the cell was derived. In addition, as more data is accumulated, the mean expression value can be revised, providing improved relative expression values based on accumulated data.

Typically denisotmetric analysis is use to determine normalized and/or relative expression levels (amounts) and is a technique known to one of skill in the art.

### III. Uses of Polymorphic Elements of the Invention

The subject polymorphisms of the invention are useful as markers in a variety of different assays. The subject polymorphisms of the invention can be used, e.g., in diagnostic assays, prognostic assays, and in monitoring clinical trials for the purposes of predicting outcomes of possible or ongoing therapeutic approaches. The results of such assays can, e.g., be used to prescribe a course of therapy after onset of ARDS, to alter an ongoing therapeutic regimen, or, more preferably, to prescribe a prophylactic course of treatment for an individual. It will be appreciated by one of skill in the art that a subject found to have a SNP(s) associated with an increased likelihood of acquiring ARDS will be more likely then subjects without the SNP(s) to develop the disease in the presence of other risk factors (e.g., trauma, burns, sepsis, etc., described above). In a preferred embodiment, a subject is tested for a particular SNP(s) in the elafin gene (e.g., A959C, A162T, or A751T) or a particular haplotype (e.g., Hap2 (TTC)). If one or more SNPs are detected then it can be predicted that the subject is more likely to develop ARDS.

It should be noted that it is possible for methods in the art to detect chromosomal variation without specifying an exact SNP site. For example, a tag SNP may be a representative SNP in a region discovered to have high linkage disequilibrium. As such, the methods of the present invention may make use of the named SNPs or other SNPs which reside nearby in the genome or are within the identified regions of linkage disequilibrium. Similarly a subject may be tested for any SNP which is found to be in Linkage disequilibrium with a particular SNP or region (e.g., any SNP which is in linkage disequilibrium with A959C, A162T, and/or A751T). If one or more such SNPs are detected then it can be predicted that the subject is more likely to develop ARDS.

The invention as it relates to polymorphisms is based, at least in part, on the discovery that certain polymorphisms are associated with an increased risk of developing ARDS. Specifically, at least three polymorphisms were discovered as being associated with an increased risk of ARDS. SNP A959C (rs2664581) with a nonsynonymous substitution of T34P within the first transglutaminase substrate domain, as well as two tagSNPs (A 162T, rs1983649; and A751T, rs6032040) in linkage disequilibrium with A959C (D's: 0.95-1.0), were found to be in Hardy-Weinberg disequilibrium. The A959C (T34P) polymorphism was significantly associated with increased risk of ARDS, while even stronger associations were observed in subjects aged<60 years. In haplotype analysis, the T7′C (162T-751T-959C) haplotype was associated with higher risk of ARDS (again with an even stronger association in subjects aged<60) when compared with the most common ATA haplotype. Furthermore, the association of the tagSNPs, A959C and A162T, and ARDS was found to be strong among subjects with extrapulmonary injury. Finding one or more of these polymorphisms in subjects at risk for developing ARDS indicates that the subject is more likely to develop ARDS than at-risk subjects who do not have the polymorphisms.

Accordingly, one aspect of the present invention relates to diagnostic assays for detecting polymorphisms, e.g., SNPs, in a biological sample (e.g., cells, fluid, or tissue) to thereby determine whether an individual is at risk of developing or predisposed to developing ARDS. In one embodiment, the methods of the invention can be characterized as comprising detecting, in a sample of cells from the subject, the presence or absence of a specific allelic variant, e.g., SNP, of the one or more polymorphic regions identified as being associated with ARDS. The allelic differences can be: (i) a difference in the identity of at least one nucleotide or (ii) a difference in the number of nucleotides, which difference can be a single nucleotide at multiple sites or several nucleotides. The invention also provides methods for detecting differences in the identified regions such as chromosomal rearrangements, e.g., chromosomal dislocation.

In some embodiments it is desirable to detect more than one SNP. For example, in some embodiments it is desirable to detect A959C, A162T, and A751T, A959C and A162T, A959C and A751T, A162T and A751T, or any combination of A959C, A162T, and A751T with one or more SNPs (e.g., PI3-1, PI3-2(rs60717610), PI3-3(rs13044826), PI3-4(rs56191952), PI3-5(rs55767422), PI3-6(rs56387543), PI3-7(rs35476703), PI3-8(rs35632684), PI3-9(rs62208416), PI3-10(rs41282752), PI3-11(rs17333103), PI3-12(rs17333180), PI3-16(rs34885285), PI3-17(rs17424474), PI3-18(rs17333381), PI3-19, PI3-20(rs34412950), PI3-21(rs35869085), PI3-22, PI3-23(rs45461302), and PI3-24(rs2267864)). The subject assays can also be used to determine whether an individual is at risk for passing on the propensity to develop a disease or disorder to an offspring. The invention also provides for prognostic (or predictive) assays for determining whether an individual is at risk of developing ARDS. The invention can also be used in prenatal diagnostics.

In another embodiment, the methods further involve obtaining a control biological sample from a control subject, determining one or more polymorphic elements in the sample and comparing the polymorphisms present in the control sample with those in a test sample.

The invention also encompasses kits for detecting the polymorphic elements in a biological sample. For example, the kit can comprise a primer capable of detecting one or more SNP sequences in a biological sample. The kit can further comprise instructions for using the kit to detect SNP sequences in the sample.

A. Detection of Polymorphisms

Practical applications of techniques for identifying and detecting polymorphisms relate to many fields including disease diagnosis.

DNA polymorphisms can occur, e.g., when one nucleotide sequence comprises at least one of 1) a deletion of one or more nucleotides from a polymorphic sequence; 2) an addition of one or more nucleotides to a polymorphic sequence; 3) a substitution of one or more nucleotides of a polymorphic sequence, or 4) a chromosomal rearrangement of a polymorphic sequence as compared with another sequence. As described herein, there are a large number of assay techniques known in the art which can be used for detecting alterations in a polymorphic sequence, most preferably the SNPs A959C (rs2664581), A162T (rs1983649), and A751T (rs6032040).

In one embodiment, analysis of polymorphisms is amenable to highly sensitive PCR approaches using specific primers flanking the sequence of interest. Oligonucleotide primers corresponding to regions associated with higher risk of ARDS can be prepared by standard synthetic techniques, e.g., using an automated DNA synthesizer. In one embodiment, detection of the polymorphism involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364). In one embodiment, genomic DNA of a cell is exposed to two PCR primers and amplification for a number of cycles sufficient to produce the required amount of amplified DNA. Commercial probe design and production kits for SNP genotyping maybe used to detect SNPs, for example TaqMan Assays (e.g., Premier Biosoft: premierbiosoft.com/tech_notes/TaqMan.html, Applied Biosystems: marketing.appliedbiosystems.com/mk/get/SNP_LANDING), and others (e.g., Marligen Biosciences: marligen.com/snp-genotyping-assays.html, Illumina:.illumina.com/pages.ilmn?ID=40, etc.).

This method can include the steps of collecting a sample of cells from a patient, isolating nucleic acid (e.g., genomic, DNA) from the cells of the sample, contacting the nucleic acid sample with one or more primers which specifically amplify a subject SNP under conditions such that hybridization and amplification of the sequence occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting polymorphisms described herein.

In one preferred embodiment, detection of single nucleotide polymorphisms (“SNP”) and point mutations in nucleic acid molecule is based on primer extension of PCR products by DNA polymerase. This method is based on the fact that the nucleoside immediately 5′ adjacent to any SNP/point mutation site is known, and the neighboring sequence immediately 3′ adjacent to the site is also known. A primer complementary to the sequence directly adjacent to the SNP on the 3′ side in a target polynucleotide is used for chain elongation. The polymerase reaction mixture contains one chain-terminating nucleotide having a base complementary to the nucleotide directly adjacent to the SNP on the 5′ side in the target polynucleotide. An additional dNTP may be added to produce a primer with the maximum of a two-base extension. The resultant elongation/termination reaction products are analyzed for the length of chain extension of the primer, or for the amount of label incorporation from a labeled form of the terminator nucleotide. (See, e.g., U.S. Pat. No. 6,972,174, the contents of which are incorporated by reference).

In one preferred embodiment, a polymorphism is detected by primer extension of PCR products, as described above, followed by chip-based laser deionization time-of-flight (MALDI-TOF) analysis, as described in, for example U.S. Pat. No. 6,602,662, the contents of which are incorporated by reference.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al, 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, after extraction of genomic DNA, amplification is performed using standard PCR methods, followed by molecular size analysis of the amplified product (Tautz, 1993; Vogel, 1997). In one embodiment, DNA amplification products are labeled by the incorporation of radiolabelled nucleotides or phosphate end groups followed by fractionation on sequencing gels alongside standard dideoxy DNA sequencing ladders. By autoradiography, the size of the repeated sequence can be visualized and detected heterogeneity in alleles recorded. In another embodiment, the incorporation of fluorescently labeled nucleotides in PCR reactions is followed by automated sequencing. (Yanagawa, T., et al., (1995). J Clin Endocrinol Metab 80: 41-5 Huang, D., et al., (1998). J Neuroimmunol 88: 192-8.

In other embodiments, polymorphisms can be identified by hybridizing a sample and control nucleic acids to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, polymorphisms can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of polymorphisms. This step is followed by a second hybridization array that allows the characterization of specific polymorphisms by using smaller, specialized probe arrays complementary to all polymorphisms detected.

In one embodiment, any of a variety of sequencing reactions known in the art can be used to directly sequence the SNPs A959C, A162Tz, A751T, or a region surrounding them to detect allelic variants, e.g., mutations, by comparing the sequence of the sample sequence with the corresponding reference sequence. Exemplary sequencing reactions include those based on techniques developed by Maxam and Gilbert (Proc. Natl. Acad Sci USA (1977) 74:560) or Sanger (Sanger et al. (1977) Proc. Nat. Acad. Sci. 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Koster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific polymorphism of can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an XBP1 allelic variant with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al. (1988) Proc. Nall Acad Sci USA 85:4397; Saleeba et al (1992) Methods Enzymol. 217:286-295. In a preferred embodiment, the control or sample nucleic acid is labeled for detection.

In another embodiment, an allelic variant can be identified by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility may be used to identify the polymorphism. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of the genome in and surrounding A959C, A162T, and A751T. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA. Several techniques based on this OLA method have been developed and can be used to detect specific allelic variants of a polymorphic region of A959C, A162Tz, and/or A751T. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In another embodiment, the single Vase polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (Cohen, D. et al. (French Patent 2,650,840; PCT Application No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Application No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

The methods described herein may be performed, for example, by utilizing pre-packaged diagnostic kits comprising at least one probe/primer nucleic acid or antibody reagent described herein, which may be conveniently used, e.g., in clinical settings to diagnose patients exhibiting symptoms or family history of a disease or illness involving a polymorphic elements. In addition, a readily available commercial service can be used to analyze samples for the polymorphic elements of the invention.

B. Primers for Amplification of Polymorphic Elements

Given that the sequences for A959C, A162T, A751T and their flanking regions are available, primers can readily be designed to amplify the polymorphic sequences and/or detect the polymorphisms by one of ordinary skill in the art. For example, the A959C polymorphism of the invention can be identified in the NCBI SNP database (using the identifiers: A959C (rs2664581), A162T(rs1983649), and A751T(rs6032040)) or by homology searching of another database containing human genomic sequences (e.g., using Blast or another program) and the location of the SNP sequence and/or flanking sequences can be determined and the appropriate primers identified and/or designed by one of skill in the art.

For example, the sequences flanking the SNPs discussed above are as follows:

rs2664581:
CTGCTGGGTG TCACCCCAGT CTGACCACTG CTCCTGAGAG ACTTGGAGTG GAGGAAGGGG
GAAGAAACAA ATACTCAAGG GAACTCTGGT CCTGTAGACC ACCCCAAAAA AGGAAGAGCC
TTCCAAGAGT GTAGCTCCCA GAGGTGTACC TTCCCTACTC AGGCCATGGT TTGAGGATGC
TGCAGTAAGC AGTGGATGGA CCCAGACCCA GAGGAAAGAC ATGGCAGCTG AAGCAGAGGC
TTACTGGGTA TAAATGTGGG CTCGTTTCTT CTTTTAACAG TTCCTGTTAA AGGTCAAGAC
M [A/C]
CTGTCAAAGG CCGTGTTCCA TTCAATGGAC AAGATCCCGT TAAAGGACAA GTTTCAGTTA
AAGGTCAAGA TAAAGTCAAA GCGCAAGAGC CAGTCAAAGG TCCAGTCTCC ACTAAGCCTG
GCTCCTGCCC CATTATCTTG ATCCGGTGCG CCATGTTGAA TCCCCCTAAC CGCTGCTTGA
AAGATACTGA CTGCCCAGGA ATCAAGAAGT GCTGTGAAGG CTCTTGCGGG ATGGCCTGTT
TCGTTCCCCA GTGAGGTGAG CACTAGCTGG AGAACGAGGA GACCCCTGAA GACACAAAAG
rs1983649:
AAAGTAGGAA AACTCTTGGG ACAATCAGAG ATGATGTGAT GTAATGTCCA TTAGTTCTTC
CTGTGAATAA TCCTGAGGGA AAGCCCCCAG GTCCCTCCCA GAATGGGGTG GATATTTCCC
AATACAGCTA AGGAATTATC CCTTGTAAAT ACCACAGACC CGCCCCTGGA GCCAGGCCAA
GCTGGACTGC ATAAAGATTG GTATGGCCTT AGCTCTTAGC CAAACACCTT CCTGACACCA
TGAGGGCCAG CAGCTTCTTG ATCGTGGTGG TGTTCCTCAT CGCTGGGACG CTGGTTCTAG
AGGCAGCTGT CACGGGAGGT GAGTGAACAG GTGACCTGCT GGGCTGGGTT GGACTAAGGG
GAGACCCTCT GGGACACCCT GGGCCAGGAC AGGGAGCACT
W [A/T]
CTGAAGCAGT AGGCAGCACT GGAGCCCAGA TTTCAGCTTT CTGTTCTTTG CCATCATATT
CAGAAAAAAT AGGACTTTGG CTGGTGGACT CCACGTGCTT TCCACCTCAG TGACTGAGAT
ATCAGGACTG TTTGTGGAAG TAATGTTGGT ATGTGGCCTT GGCCTCAGAT GTCAATACCT
GTGCAGAATG TGCAATAAAA TAATGAACTC CAGGATTTTA AACCTTGGGT GTGGACACAG
TCCCCCGTTT CTCTGCCCCA TAAAAGCACT GGAGTAATCA GTACTCTAAA AGGAGGTTAA
GAAACAACAA GCCTTCAGGA ATCATGTTGT TTGAGGACCC CCATTTTATA AGGAGGGAAC
CAAAAATGTA GAAATGAGTG AGCAATTGCC AAGGTAATTC
rs6032040:
TGTGCAATAA AATAATGAAC TCCAGGATTT TAAACCTTGG GTGTGGACAC AGTCCCCCGT
TTCTCTGCCC CATAAAAGCA CTGGAGTAAT CAGTACTCTA AAAGGAGGTT AAGAAACAAC
AAGCCTTCAG GAATCATGTT GTTTGAGGAC CCCCATTTTA TAAGGAGGGA ACCAAAAATG
TAGAAATGAG TGAGCAATTG CCAAGGTAAT TCCCAGAGCC AGGATGGGGC TCAAGTCTCC
TAGTATGTGG CTCAGGGTTC TTTCCTACTC CAATGCACTT CCTAACAAAT GACAATGTGT
CCTCTTCACT GCTGGGTGTC ACCCCAGTCT GACCACTGCT CCTGAGAGAC TTGGAGTGGA
GGAAGGGGGA AGAAACAAAT ACTCAAGGGA ACTCTGGTCC
W [A/T]
GTAGACCACC CCAAAAAAGG AAGAGCCTTC CAAGAGTGTA GCTCCCAGAG GTGTACCTTC
CCTACTCAGG CCATGGTTTG AGGATGCTGC AGTAAGCAGT GGATGGACCC AGACCCAGAG
GAAAGACATG GCAGCTGAAG CAGAGGCTTA CTGGGTATAA ATGTGGGCTC GTTTCTTCTT
TTAACAGTTC CTGTTAAAGG TCAAGACACT GTCAAAGGCC GTGTTCCATT CAATGGACAA
GATCCCGTTA AAGGACAAGT TTCAGTTAAA GGTCAAGATA AAGTCAAAGC GCAAGAGCCA
GTCAAAGGTC CAGTCTCCAC TAAGCCTGGC TCCTGCCCCA TTATCTTGAT CCGGTGCGCC
ATGTTGAATC CCCCTAACCG CTGCTTGAAA GATACTGACT
*Note that the symbols M and W are used in the above sequences
to indicate the polymorphism. “M” indicates the existence
of A/C alleles. “W” similarly indicates A/T alleles.

Using the sequences provided above, one of skill in the art can readily design oligonucleotides to amplify and/or detect the polymorphism within these sequences.

In one embodiment, a primer for amplification of a polymorphic elements is at least about 5-10 base pairs in length. In one embodiment, a primer for amplification of a polymorphic elements is at least about 15-20 base pairs in length. In one embodiment, a primer for amplification of a polymorphic element is at least about 20-30 base pairs in length. In one embodiment, a primer for amplification of a polymorphic element is at least about 30-40 base pairs in length. In one embodiment, a primer for amplification of a polymorphic element is at least about 40-50 base pairs in length. In one embodiment, a primer for amplification of a polymorphic elements is at least about 50-60 base pairs in length. In one embodiment, a primer for amplification of a polymorphic elements is at least about 60-70 base pairs in length. In one embodiment, a primer for amplification of a polymorphic elements is at least about 70-80 base pairs in length. In one embodiment, a primer for amplification of a polymorphic elements is at least about 80-90 base pairs in length. In one embodiment, a primer for amplification of a polymorphic elements is at least about 90-100 base pairs in length. In one embodiment, a primer for amplification of a polymorphic elements is at least about 100-110 base pairs in length. In one embodiment, a primer for amplification of a polymorphic elements is at least about 110-120 base pairs in length. In one embodiment, a primer for amplification of a polymorphic elements is at least about 120-130 base pairs in length. In one embodiment, a primer for amplification of a polymorphic elements is at least about 130-140 base pairs in length. In one embodiment, a primer for amplification of a polymorphic elements is at least about 140-150 base pairs in length. In one embodiment, a primer for amplification of a polymorphic elements is at least about 150-160 base pairs in length. In one embodiment, a primer for amplification of a polymorphic elements is at least about 160-170 base pairs in length. In one embodiment, a primer for amplification of a polymorphic elements is at least about 170-180 base pairs in length. In one embodiment, a primer for amplification of a polymorphic elements is at least about 180-190 base pairs in length. In one embodiment, a primer for amplification of a polymorphic elements is at least about 190-200 base pairs in length.

In one embodiment, a primer for amplification of a polymorphic element of the invention is located at least about 200 base pairs away from (upstream or downstream of) the polymorphism to be amplified (i.e., leaving about 200 nucleotides from the end of the primer sequence to the polymorphism). In another embodiment, a primer for amplification of a polymorphism of the invention is located at least about 150 base pairs away from (upstream or downstream of) the polymorphic sequence to be amplified. In another embodiment, a primer for amplification of a polymorphism of the invention is located at least about 100 base pairs away from (upstream or downstream) of the polymorphic sequence to be amplified. In another embodiment, a primer for amplification of a polymorphism of the invention is located at least about 75 base pairs away from (upstream or downstream of) the polymorphic sequence to be amplified. In another embodiment, a primer for amplification of a polymorphism of the invention is located at least about 50 base pairs away from (upstream or downstream of) the polymorphic sequence to be amplified. In another embodiment, a primer for amplification of a polymorphism of the invention is located at least about 25 base pairs away from (upstream or downstream of) the polymorphic sequence to be amplified. In another embodiment, a primer for amplification of a polymorphism of the invention is located at least about 10 base pairs away from (upstream or downstream of) the polymorphic sequence to be amplified. In another embodiment, a primer for amplification of a polymorphism of the invention is located at least about 5 base pairs away from (upstream or downstream of) the polymorphic sequence to be amplified. In another embodiment, a primer for amplification of a polymorphism of the invention is located at least about 2 base pairs away from (upstream or downstream of) the polymorphic sequence to be amplified. In yet another embodiment a primer for amplification of a polymorphism of the invention is adjacent to the polymorphic sequence to be amplified.

### IV. Methods of Treatment

As described herein, the amount of elafin in a subject that develops ARDS is significantly higher than in a subject who does not develop ARDS. This amount of elafin decreases as the subject progresses from the acute phase of ARDS to the recovery phase of ARDS. Since elafin has been shown to be anti-inflammatory in cases of pulmonary bacterial infection or acute lung injury and the amount of elafin is inversely correlated with the development of ARDS, this indicates a protective role of elafin in the pathophysiologic response to lung injury seen in ARDS. Thus, an increased amount of elafin would be useful for treating ARDS.

Accordingly, the present invention also provides for both prophylactic and therapeutic methods of treating a subject, e.g., a human, who has, is at risk of, or is susceptible to (e.g. has a prior history of) ARDS. As used herein, “treatment” of a subject includes the application or administration of a therapeutic agent to a subject, or application or administration of a therapeutic agent to a cell or tissue from a subject, who has ARDS, has a symptom of ARDS, or is at risk of (or susceptible to) ARDS, with the purpose of curing, inhibiting, healing, alleviating, relieving, altering, remedying, ameliorating, improving, or affecting the ARDS, a symptom of the ARDS, or the risk of (or susceptibility to) ARDS.

As used herein, a “therapeutic agent” or “compound” includes any compound or agent capable of modulating the expression and/or activity of elafin. Such compounds include, but are not limited to, small molecules, peptides, peptidomimetics, and polypeptides incorporated into pharmaceutical compositions suitable for administration. In preferred embodiments, the compound is elafin protein, an elafin peptide or peptide mimetic.

Pharmaceutical compositions for use in the therapeutic methods of the invention typically comprise the compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The therapeutic methods of the invention involve administering to a subject a compound capable of increasing the expression, amount, and/or activity of elafin in an amount effective to increase the expression, amount, and/or activity of elafin. Such compounds may, for example, be identified using cell based or cell free screening assays known in the art (i.e., including elafin activity assays). Test compounds may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145). Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer. The aerosol may be prepared and delivered according to any method known in the art including, but not limited to, the methods and preparations disclosed in U.S. Pat. Nos. 5,479,920, 5,225,183, 5,388,574, 5,404,871, 4,174,295, 5,049,388, 4,819,629, 5,099,861, 5,617,844, 5,231,983, 5,126,123, 4,823,784, 4,819,665, 3,789,843, 4,592,348, 5,605,674, 5,394,866, 3,565,070, 5,376,359, 3,838,686, 3,636,949, 5,695,743 5,479,920, 6,460,537, 5,069,204, and 4,941,483 which are incorporated herein by reference.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. Nos. 4,522,811, 4,356,167, 4,551,288, 4,873,088, 5,023,087, 4,963,367, 4,946,683, 5,580,575, 6,041,252, 5,543,152, 5,770,222, 5,718,914, 4,895,719, 4,761,288, 5,576,016, 6,228,393.

Elafin may be administered by any other method known in the art which will increase elafin in the lungs of ARDS patients. Substances which increase expression of elafin may be administered by the methods mentioned above or via other routes such as transdermal patches, capsules, or tablets where appropriate. In addition, the methods of this invention may be combined with the traditional treatment regimes applied to subjects with ARDS. Typically, supportive care is given to subjects with ARDS. This may include providing fluids, providing appropriate nutritional support, administration of sedatives and analgesia (e.g., administration of morphine), control of blood glucose levels (e.g., by nutritional adjustment or administration of insulin). Evaluation and aggressive treatment for conditions such as sepsis or pneumonia may also be conducted. Oftentimes, subjects are subjected to mechanical ventilation or given higher concentrations of oxygen to assist breathing. In addition, clinicians and scientists have recently been experimenting with treatments such as inhalable surfactant therapy, vasodilators (e.g., nitric oxide), and glucocorticoids and other anti-inflammatories, which may be administered at the discretion of a clinician. A brief review and analysis of treatment strategies may be seen in (Ware L B, Matthay M A. The acute respiratory distress syndrome. N Engl J Med. 2000 May 4; 342(18):1334-49.) which is incorporated herein by reference.

### VI. Kits

The invention also provides compositions and kits for predicting whether a subject at risk of developing ARDS will develop ARDS and for monitoring the efficacy of a treatment regimen for ARDS. These kits include one or more of the following: a detectable antibody that specifically binds to elafin, an oligonucleotide probe which binds to elafin DNA or mRNA, a detectable antibody that specifically binds to HNE, an oligonucleotide probe which binds to HNE DNA or mRNA, reagents for obtaining and/or preparing body samples, and instructions for use. Kits can also include instructions for interpreting the results obtained using the kit.

The invention also encompasses kits for detecting the polymorphic elements in a biological sample. For example, the kit can comprise a primer capable of detecting one or more SNP sequences in a biological sample. The kit can further comprise instructions for using the kit to detect SNP sequences in the sample.

The kits of the invention may optionally comprise additional components useful for performing the methods of the invention. By way of example, the kits may comprise fluids (e.g., SSC buffer) suitable for annealing complementary nucleic acids or for binding an antibody with a protein with which it specifically binds, one or more sample compartments, an instructional material which describes performance of a method of the invention and specific tissue or fluid sample controls/standards.

For antibody-based kits, the kit can comprise, for example: (1) a first antibody (e.g., attached to a solid support) which binds to an elafin polypeptide; and, optionally, (2) a second, different antibody which binds to either elafin or the first antibody and is conjugated to a detectable label. These kits may further comprise a third antibody which binds to an HNE polypeptide; and, optionally, a fourth antibody which binds to either HNE or the third antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding elafin or (2) a pair of primers useful for amplifying elafin. These kits may further comprise, for example, (3) an oligonucleotide, e.g., a detectably labeled oligonucleotide, which hybridizes to a nucleic acid sequence encoding HNE or (4) a pair of primers useful for amplifying HNE. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

A kit may also comprise an oligonucleotide that specifically detects a polymorphism in or near the elafin gene. For example, a kit may include oligonucleotides that specifically detect PI3-1, PI3-2(rs60717610), PI3-3(rs13044826), PI3-4(rs56191952), PI3-5(rs55767422), PI3-6(rs56387543), PI3-7(rs35476703), PI3-8(rs35632684), PI3-9(rs62208416), PI3-10(rs41282752), PI3-11(rs17333103), PI3-12(rs17333180), A162T(PI3-13; rs1983649), A751T (PI3-14; rs6032040), A959C(PI3-15; rs2664581), PI3-16(rs34885285), PI3-17(rs17424474), PI3-18(rs17333381), PI3-19, PI3-20(rs34412950), PI3-21(rs35869085), PI3-22, PI3-23(rs45461302), PI3-24(rs2267864), or any of the SNPs described herein. The kit may include multiple oligonucleotides to detect one or more of the SNPs described herein. For example, a kit may include oligonucleotides to detect A162T, A751T, and A959C, oligonucleotides to detect A162T and A959C, oligonucleotides to detect A162T and A751T, oligonucleotides to detect A751T and A959C, or oligonucleotides to detect A162T, A751T, A959C, and one or more other SNPs. Kits to detect polymorphisms may include oligonucleotides which hybridize to a polymorphism sequence in vivo, in vitro, or in situ (e.g., an oligonucleotide which hybridized to the sequence of A162T, A751T, or A959C)

The invention is further illustrated by the following examples, which should not be construed as further limiting. The contents of all references, pending patent applications and published patents, cited throughout this application are hereby expressly incorporated by reference

### EXAMPLES

### Example 1

### Material and Methods

### Study Subjects

The studies described herein were conducted within the ongoing Molecular Epidemiology of ARDS project at the Massachusetts General Hospital (MGH) and Harvard School of Public Health, both in Boston, Mass. These studies were initiated in 1999, and approved by the Human Subjects Committees of both institutions. Study subjects were recruited from subjects admitted to one of the four adult intensive care units (ICU) at MGH as described previously in Michelle Ng Gong et al. (The European Respiratory Journal. 26 (3), 382-9 Sep. 2005.) and Michelle Ng Gong et al. (Chest. 125 (1), 203-11 Jan. 2004). Eligible subjects were individuals admitted to the ICU with at least one risk factor for the development of ARDS: 1) sepsis, 2) septic shock, 3) trauma, 4) pneumonia, 5) aspiration, or 6) massive transfusion of packed red blood cells (PRBC: defined as greater than 8 units of PRBC during the 24 hours prior to admission). Subjects were followed prospectively during ICU for the development of ARDS. Subjects who developed ARDS, defined by the American European Consensus Committee (AECC) criteria (Bernard, G. R., et al. 1994. Am J Respir Crit Care Med 149:818-824.), were identified as ARDS cases. Controls were identified as at-risk subjects who did not meet criteria for ARDS during their stay in the ICU and had no prior history of ARDS. Baseline clinical information, vital signs, and laboratory testing results in the first 24 hours after ICU admission were collected for calculation of the Acute Physiological and Chronic Health Evaluation (APACHE) III score (Knaus, W. A., et al. 1991. Chest 100:1619-1636.). The date of ARDS diagnosis was defined as Day 0 of ARDS, the dates after Day 0 were referred as plus days (Day +1, Day +2, etc), and the dates prior Day 0 were identified as minus days (Day −1, Day −2, etc). A written informed consent was obtained from each subject or an appropriate proxy of the subject.

### RNA Sample Preparation and Microarray Hybridization

A self-control (paired sampling) study design was applied in microarray analyses using whole blood total RNA samples as described previously (Zhaoxi Wang et al. Environmental health perspectives. 113 (2), 233-41 Feb. 2005). Briefly, after the blood was drawn, TRI Reagent BD (Molecular Research Center, Inc., Cincinnati, Ohio) was added and mixed to stabilize the whole-blood total RNA. The stabilized samples were transported to the laboratory on dry ice and stored at −80° C. until RNA extraction. Total RNA was isolated later from 10 mL of whole blood according to manufacturer protocols and purified using the RNeasy mini kit (Qiagen, Chatsworth, Calif.). The yield and quality of RNA were assessed by spectrophotometry and the Agilent 2100 Bioanalyzer (Agilent Technologies, Palo Alto, Calif.).

To investigate ARDS-related gene expression changes, two blood samples were collected from each ARDS cases, who participated in the exploratory gene expression study. The first sample was collected between Day 0 and Day 3 of ARDS diagnosis, corresponding to the acute-stage of ARDS. The second sample was collected prior to the day of discharge from the ICU, representing the recovery-stage of ARDS. Protocols for sample collection and processing, whole blood total RNA extraction, and quality assessment were described previously (Zhaoxi Wang et al. Environmental health perspectives. 113 (2), 233-41 February 2005). RNA samples were hybridized to Affymetrix Human Genome U133A GeneChips (Affymetrix, Santa Clara, Calif.) at the Microarray Core Facility of the Dana-Farber Cancer Institute (Boston, Mass.). The paired RNA samples collected from each subject were processed together in one batch of microarray analysis to minimize inherent variations. After initial quality check, raw microarray signals were normalized across all microarrays and the model-based expression values were calculated using DNA-Chip Analyzer 2006 (dChip, dchip.org/) software (Li, C., and Wong, W. H. Proc Natl Acad Sci U S A 98:31-36. 2001). The Detection Calls of a gene (Present Calls or Absent Calls) in an RNA sample was carried out by Affymetrix MAS 5.0 software using the one-sided Wilcoxon's signed-ranked algorithm (Liu, W. M., et al. Bioinformatics 18:1593-1599. 2002.).

### Microarray Data Analysis

Out of a total of 22,215 probe sets on Affymetrix U133A microarray, a subset of 6,772 probe sets with Present Calls in over 50% of tested arrays was used in microarray data analysis. Genes with altered expression between two time points were identified by initially screening for average fold changes of paired samples larger than 1.2 or less than −1.2, and then tested genes using a one-sided paired t-test with a p-value<0.05 as the cutoff for significance. Functional clustering analyses of the identified genes were carried out on the annotations defined by the Gene Ontology Consortium (GO, geneontology.org/) (M. Ashburner et al. Nat Genet 25:25-29. 2000.), using a non-redundant gene list for Affymetrix UI 33A microarray in the analysis (downloaded from Affymetrix website on Mar. 7, 2007). GeneNotes software (combio.cs.brandeis.edu/GeneNotes/index.htm) was used to identify GO biological process significantly enriched with the altered genes, with Bonferroni correction (Pengyu Hong and Wing H Wong. BMC bioinformatics. 6, 20 2005).

### Quantitative Real-Time PCR Analysis Using Taqman® Assays

To validate the microarray data, TaqMan® (Applied Biosystems, Foster City, Calif.) quantitative real-time reverse transcriptase polymerase chain reaction (RT-PCR) was performed on available paired RNA samples from 6 subjects. TaqMan® Probes, available as “Assay on Demand”, were used in the analyses of the expression levels of five target genes, including CYP4F3 (Hs00168521_m1), HPGD (Hs00168359_m1), IL8 (Hs00174103_m1), MMP9 (Hs00234579_m1), and PI3 (Hs00160066_m1), as well as 3 endogenous control genes (18S, Hs99999901_s1; ACTB, Hs99999903_m1; and RPLP0, Hs99999902_m1), according to the manufacturer suggested procedures. Briefly, cDNA was first synthesized from ˜2 μg RNA in a 100 μL reaction volume, using the High Capacity cDNA Archive kit (Applied Biosystems, Foster City, Calif.). Quantitative RT-PCR was then performed on synthesized cDNA in triplicates on an ABI PRISM 7900HT Sequence Detection System (Applied Biosystems, Foster City, Calif.). Of three simultaneously detected internal control genes, RPLP0 showed the lowest level of intra-assay variations, and was chosen to normalize RT-PCR results. Relative expression levels between paired RNA samples were determined using the ΔΔCt method with the value the first RNA sample (acute-stage of ARDS) of each pair as the calibrator (K J Livak and T D Schmittgen. Methods (San Diego, Calif.) 25 (4), 402-8. December 2001.).

Analysis of PI3 (pre-elafin) and MMP-9 Levels in Plasma

A nested case-control study of plasma levels of PI3 and MMP-9 was conducted on 63 subjects, including 40 ARDS subjects and 23 controls. From the ongoing Molecular Epidemiology of ARDS project, a total of 126 ARDS subjects had plasma samples available for the analysis, with 94% of them having only one plasma sample. Most of the plasma samples (94%) were collected during the first 7 days after ICU admission. Three groups of ARDS cases were selected based the date of sample collection relative to the ARDS diagnosis date, including pre-diagnosis group (n=12, Day −5 to Day −1), day of diagnosis group (n=10, Day 0), and post-diagnosis group (n=18, Day 1 to Day 3). All at-risk controls provided paired plasma samples with the first sample collected during the first two days of ICU admission and a second sample collected three days later. Demographic characterization of these subjects is shown in Table 4. Most of descriptive characteristics were not significantly different between ARDS cases and controls, except that ARDS cases were more frequently received packed red blood cell transfusion (p=0.009). In addition, for both ARDS subjects and at-risk controls, there was no statistically significant difference in baseline characteristics between the selected subjects and the rest of the study population. Plasma samples were stored at −80° C. until analysis. Plasma PI3 and MMP9 levels were quantified in duplicate using Human pre-ELAFIN/SKALP ELISA Test Kit (Cell Sciences, Canton, Mass.) and Human MMP-9 Immunoassay Kit (Millipore, Billerica, Mass.), according to the manufacturer's recommended protocol.

### Statistical Analysis

The baseline characteristics between groups were compared using chi-square tests for categorical variables and by student t-test for normally distributed continuous variables. The correlations between microarray expression data and quantitative RT-PCR data were estimated by Spearman correlation test. All statistical analyses were performed using the SAS statistical software package (version 9.1, SAS Inc., Cary, N.C.).

### Literature Mining Using Medgene and BioGene Database for Genes Related to ARDS

MedGene and BioGene are literature mining projects sponsored by Harvard Institute of Proteomics (hip.harvard.edu) for the creation of human gene-to-disease co-occurrence network of all named human genes and all human diseases by automated analysis of MeSH indexes, title and abstracts in millions of Medline records (Hu, Y., et al. 2003. J Proteome Res 2:405-412.). A web-based interface of MedGene database (by Mar. 14, 2007) hosted at Harvard Institute of Proteomics was used to conduct literature mining for genes associated with ARDS (MeSH disease term: Respiratory Distress Syndrome, Adult) in the Medline Database. The BioGene database (by Mar. 14, 2007) was also searched for genes associated with ARDS-related MeSH vocabularies, including neutrophil, leukotriene, and prostaglandin. The statistical method used to rank the gene list was the product of frequency. The database only allowed downloading maximum number of 100 top-ranked genes for each term of disease or MeSH vocabulary, without specific request for longer gene list. The numbers of top-ranked genes of MeSH mining are the combined lists of non-redundant genes of all sub-vocabularies available at BioGene. In addition, MedGene was used to sort the linkage of genes identified by paired t-tests and ARDS into four categories, including directly link (first-degree association), directly linked by gene family term, indirect link through other ARDS genes, and not previously associated with ARDS.

### Results

High-quality microarray data of paired RNA samples were obtained from 11 subjects. Baseline characteristics for these subjects are shown in Table 1. Although only ARDS subjects were recruited for microarray analysis, two subjects, who were misclassified originally as ARDS subjects, did not meet the AECC criteria for ARDS while in ICU and therefore were excluded from the paired microarray analysis. In addition, one ARDS case was excluded since the acute-stage sample was collected two days prior to the diagnosis of ARDS. ARDS-related gene expression changes were examined among the eight remaining pairs of RNA samples, corresponding to acute-stage (Day 0 to Day 3 of ARDS diagnosis) and recovery-stage (six-day period between three days before and three days after ICU discharge) of ARDS. The median period between two sample collections was 15 days, with a range from 2 days to 24 days as shown in Table 1.

Comparisons of gene expression between the acute-stage and the recovery-stage of ARDS were performed using a straightforward approach of fold-change ranking plus a p-value cutoff (<0.05), which was proved to be a more reliable ranking criterion for gene selection in previous studies (Shi, L., et al. 2006. Nat Biotechnol 24:1151-1161; and Guo, L., et al. 2006. Nat Biotechnol 24:1162-1169.). From a subset of 1,123 probe sets with an average paired fold-change larger than 1.2, 126 genes (136 probe sets) were identified with significantly altered gene expression. Compared to the recover-stage of ARDS, 70% (n=88) of genes were expressed at lower levels and 30% (n=38) were expressed at higher levels during the acute-stage, showing that many genes were suppressed during the acute-stage of ARDS (Table 5 for complete list of genes). A subset of 27 genes with large changes of expression (greater than 1.5 fold-change) as shown in Table 2, as compared with previous microarray analyses of whole blood paired RNA samples was also found (Wang, Z., et al. 2005. Environ Health Perspect 113:233-241.). Of the 27 genes listed with a greater than 1.5 fold-change in expression, gene PI3, IL8, and HGPD had two probe sets which showed significant ARDS-related variations.

The biological functions that are associated with the genes demonstrating ARDS-related expression changes were next investigated by GO Biological Process categories and by limited literature mining in the Medline database. The GO analysis focused on GO terms with over-representation of genes which were differentially expressed more frequently than expected by chance. It was found that all GO terms, which tested significantly after Bonferroni correction (p<0.0002) for multiple comparison, were closely interconnected on GO structure under two general GO categories: Response to Stimulus (GO: 50896) and Death (GO: 16265), as listed in Table 3. In addition, a more specific GO category of Prostaglandin Metabolism (GO: 6693) was marginally significant after Bonferroni correction (p=0.0004), with three genes (CD74, HGPD, and PTGS2) associated with ARDS-related expression changes. A total of 44 genes were identified within three categories of GO terms: Response to Stimulus (GO: 50896), Death (GO: 16265), and Prostaglandin Metabolism (GO: 6693). In addition, the list of 126 altered genes was compared with the results of MedGene literature mining in Medline database for top-ranked genes linked to ARDS, as well as ARDS-related MeSH terms, including “neutrophil”, “leukotriene” and “prostaglandin” (Hu, Y., et al. 2003. J Proteome Res 2:405-412.). Three genes associated with ARDS and an additional 27 genes associated with relevant MeSH terms (Table 6), of which 20 genes were also found in the significant GO categories were identified. Furthermore, using MedGene literature mining of the 126 gene with altered expression, 8 genes were found directly linked to the development of ARDS, and all were located in the GO term of Response to Stimulus (GO: 50896) (Table 5).

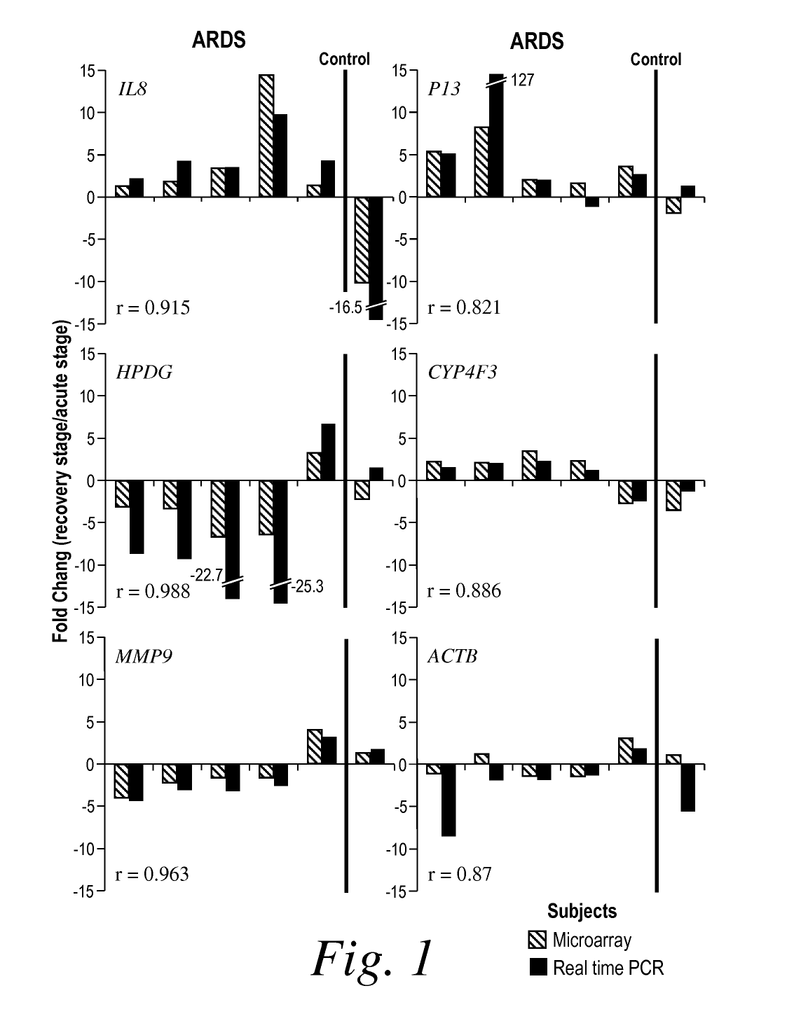

Microarray results were validated by analyzing five selected genes using quantitative RT-PCR on six subjects with enough paired RNA samples available after microarray analysis. Three genes, the down-regulated PI3 and IL8 genes and up-regulated HPGD gene at the acute-stage of ARDS, were chosen based on their large differential expression between acute-stage and recovery-stage of ARDS, and being identified repeatedly in Medline literature mining. In addition, a gene with marginal expression change (CYP4F3, −1.5 fold-change, p=0.08; encoding enzyme involved in the process of inactivating and degrading leukotriene B4) and a gene with no change (MMP9, 1.0 fold-change, p=1.0; encoding a matrix metalloproteinase) were chosen as reference genes. As shown in FIG. 1, the microarray and quantitative RT-PCR results showed good agreement in fold-changes between two time points (correlation coefficients: 0.82-0.99). For all tested genes, there were no statistically significant differences between microarray and RT-PCR measured fold-changes by paired t-test (p range 0.052-0.803).

Among 126 genes identified from paired t-test, the PI3 gene had the largest fold-change in expression between the acute-stage and the recovery-stage of ARDS (−2.98 fold-changes). This gene encodes a neutrophil elastase inhibitor, peptidase inhibitor 3 (PI3), formally called elafin. PI3 is one of two low-molecular-weight protease inhibitors of the anti-leukoprotease family, which was previous reported to be localized to the injury sites (Sallenave, J. M. 2000. Respir Res 1:87-92.). In addition to antiproteinase activity, PI3 proteins also demonstrate antimicrobial and anti-inflammatory activities (Roghanian, A., et al. 2006. Am J Respir Cell Mol Biol 34:634-642.). Compared with the recovery-stage of ARDS, the PI3 gene was expressed at a 2.98 fold-change lower level in peripheral blood during the acute-stage of ARDS (Table 2), showing that PI3 gene product plays important roles in the development of ARDS.

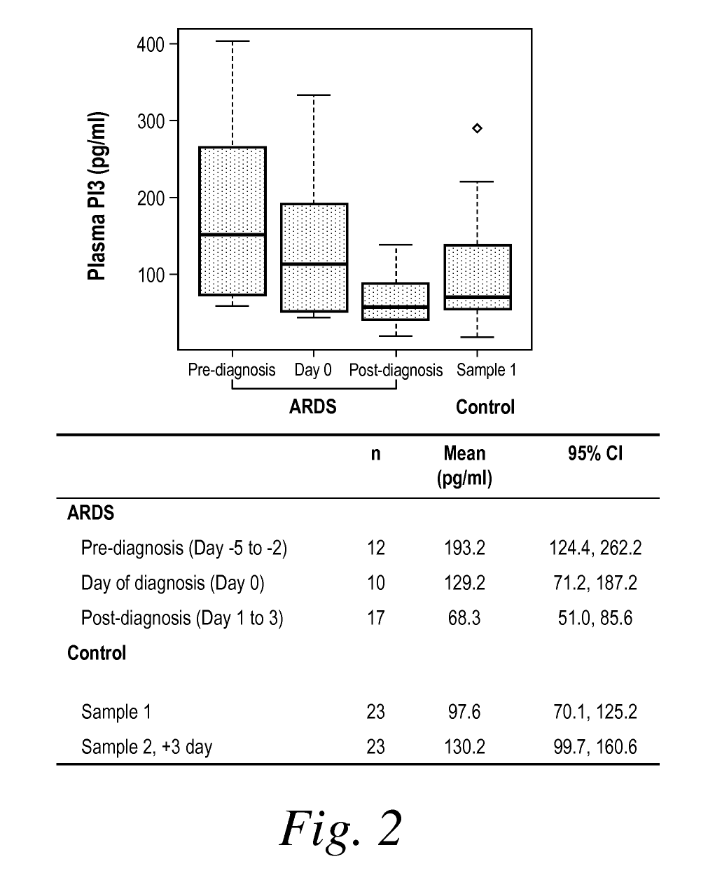

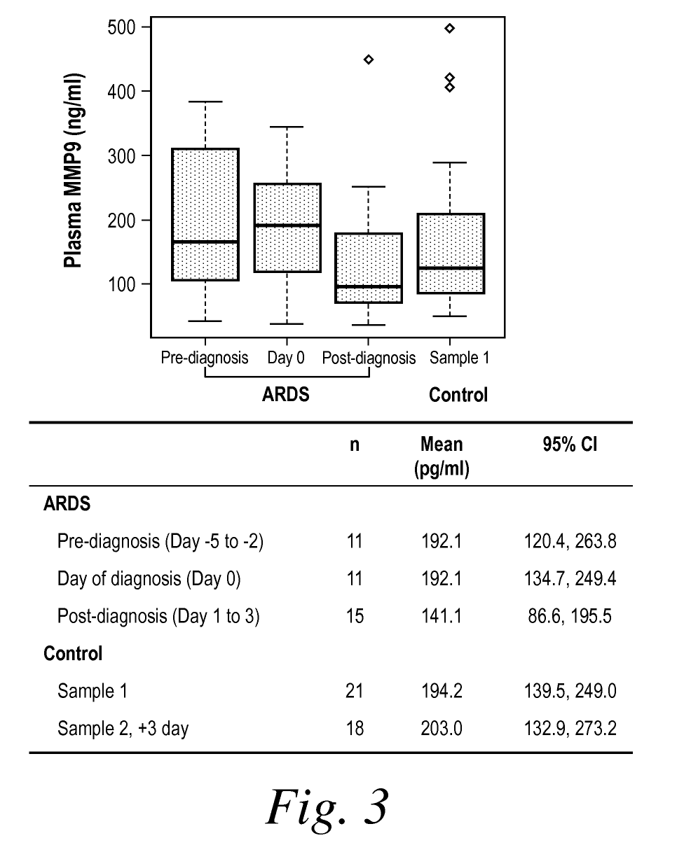

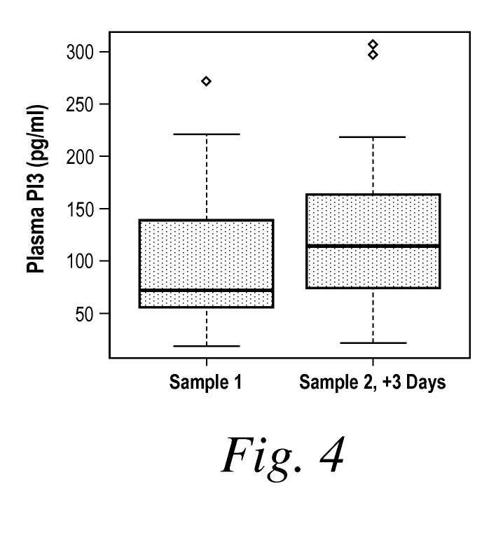

The role of PI3 in ARDS was further analyzed by comparing plasma pre-elafin (fully functional precursor of elafin) levels among the pre-diagnosis group (day −5 to day −1), day-of-diagnosis group (within 24 hours of diagnosis), and post-diagnosis group (day 1 to day 3). All plasma samples were collected from the same cohort of Molecular Epidemiology Study of ARDS (see Methods). There was a decrease in plasma PI3 levels from pre-diagnosis group to post-diagnosis group as shown in FIG. 2. The post-diagnosis group had the lowest plasma PI3 level, which was statistically significantly lower than the pre-diagnosis group (p=0.0009) and marginally significantly lower than the day-of-diagnosis group (p=0.05). Plasma PI3 levels in the post-diagnosis group were not significantly different from that of at-risk controls collected during the first 48 hours of ICU admission (p=0.19). Among three ARDS groups, the pre-diagnosis group had the highest plasma PI3 level, which was also significantly higher than the first samples of the controls collected during the first 48 hours of ICU admission (p=0.01). In contrast, no statistically significant difference existed in plasma levels of MMP-9 between ARDS cases and at-risk controls as expected based on the lack of change (1.0 fold-change, p=1.0) in the microarray analysis (FIG. 3). In addition, in the controls a statistically significant increase of plasma PI3 levels (mean ratio=1.53, 95% CI, 1.28-1.78; p=0.0007) was found three days after the first 48 hours of ICU admission using paired t-test (FIG. 4).

Using a microarray based global gene profiling of whole blood total RNA, 126 genes with altered expression were between the acute-stage (Day 0 to Day 3 of ARDS diagnosis) and recovery-stage (six-day period between three days before and three days after ICU discharge) were identified in subjects with ARDS. Based on Gene Ontology annotations, the ARDS-related gene expression changes were clustered in GO biological processes related to Response to Stimulus (GO: 50896) and Death (GO: 16265). Significant subordinate GO terms of Death (GO: 16265) included Cell Death (GO: 8219), Programmed Cell Death (GO: 12501), Apoptosis (GO: 6915), Regulation of Programmed Cell Death (GO: 43067), and Regulation of Apoptosis (GO: 42981). This clustering of differentially expressed genes related to the regulation of apoptosis is would support previous observations of reduced apoptosis in alveolar neutrophils isolated from ARDS subjects were previously reported (G. Matute-Bello et al. American journal of respiratory and critical care medicine. 156 (6), 1969-77 December 1997).

There is a significant overlap in the gene expression alterations observed in ARDS subjects in this study as previously published in human volunteers with endotoxin-induced pulmonary inflammation using the same Affymetrix U133A GeneChips and a similar paired-sampling study design (Christopher D. Coldren et al. AJP—Lung Cellular and Molecular Physiology. 291 (6), L1267-1276 01 Dec. 2006). Coldren et al. compared the gene expression in the alveolar neutrophils isolated from bronchoalveolar lavage (BAL) fluid 16 hours after bronchoscopic instillation of endotoxin with the gene expression in the circulating neutrophil isolated from peripheral blood pre- and post-endotoxin challenge. They found large gene expression differences, with approximately 15% of expressed genes having altered expression. Since bronchoscopic instillation of endotoxin only caused transient and local pulmonary inflammation in the healthy volunteers, Coldren et al. observed fewer gene expression variations (˜1% of expressed genes) between pre- and post-endotoxin instillation circulating neutrophils. Forty-seven of the 126 genes (37%) with altered gene expression in the present study were also found in Coldren's study. Moreover, 14 of 27 genes (52%) with large changes of gene expression (greater than 1.5 fold-changes) in the acute-stage compared with recovery-stage of ARDS, including PI3 and IL8, were also noted by Coldren et al. (Table 2). The similarity of gene expression profiles between this study and those observed in human model of pulmonary inflammation provide potentially valuable insight of the roles of the altered genes in the development of ARDS.

Among 126 identified genes, the gene PI3 had the largest expression variation in peripheral blood of ARDS subjects. Expression of PI3 gene was suppressed at the early, acute-stage of ARDS compared with significantly higher levels during the recovery-stage of ARDS. PI3 gene encodes protein peptidase inhibitor 3 with two isoforms, including a 95-amino acid molecule named pre-elafin (also known as trappin-2) and a 58-amino acid molecule called elafin, which was produced by proteolytic cleavage of pre-elafin. Both pre-elafin and elafin have two functional domains, including the C-terminal domain containing the antiproteinase active site and the N-terminal domain containing motifs of transglutaminase substrate. The transglutaminase substrate motifs, five motifs in pre-elafin molecules and one motif in elafin, enables covalent binding of proteinase inhibitors to the extracellular matrix (ECM) proteins (Schalkwijk, J., et al. 1999. Biochem J 340 (Pt 3):569-577.). PI3 Expression is induced by inflammation-initiating cytokines and localized to the site of inflammatory response, including airways, skin, and other mucosal surfaces (Sallenave, J. M., et al. 1994. Am J Respir Cell Mol Biol 11:733-741; Alkemade, J. A., et al. 1994. J Cell Sci 107 (Pt 8):2335-2342; and Pfundt, R., et al. 1996. J Clin Invest 98:1389-1399.). However, this study found that the expression of PI3 gene in peripheral blood was differentially expressed during the acute-stage of ARDS compared with the recovery-stage.

Importantly, the microarray findings of lower expression of the PI3 gene during the acute-stage of ARDS is consistent with the results of the ELISA assay measurements of plasma PI3 levels, which found PI3 was expressed at the lowest level in plasma during the acute-stage (Day 1 to Day 3 of ARDS diagnosis), compared with PI3 levels prior and the day of ARDS diagnosis. The time course of PI3 changes in plasma is well correlated with the course of ARDS development. Before the onset of ARDS (Day −5 to Day −1 of ARDS diagnosis), the plasma PI3 levels in ARDS subjects are significantly increased (mean 193.2 pg/ml, 95% C, 124.4-262.2 pg/ml; p=0.01) compared with at-risk controls within the first 48-hour ICU admission (mean 97.6 pg/ml, 95% C, 70.1-125.2 pg/ml). During the course of ARDS development, the elevated plasma PI3 levels were decreased. In contrast, there was a significant but moderate increase of plasma PI3 levels during a 3-day period after ICU admission in at-risk controls (FIG. 4). This demonstrates that PI3 levels in plasma can be used as a biomarker for monitoring the development and clinical progress of ARDS. Similarly, an earlier study reported that the serum levels of elafin could be used to monitor the disease activity of psoriasis, an inflammatory skin and joint disease (H A Alkemade et al. The Journal of investigative dermatology. 104 (2), 189-93 February 1995). The serum elafin levels were correlated with the clinical course of psoriasis and disease severity score (Psoriasis Area Severity Index), and a decrease in serum elafin levels was associated with response to cyclosporine A treatment Neutrophils play a crucial role in the initiation and propagation of ARDS (Weiland, J. E., et al. 1986. Am Rev Respir Dis 133:218-225.). Considerable evidence exists for the role of neutrophil-derived proteinases in the pathogenesis of ARDS, including neutrophil elastase and collagenase (Lee, C. T., et al. 1981. N Engl J Med 304:192-196; McGuire, W. W., et al. 1982. J Clin Invest 69:543-553; and Christner, P., et al. 1985. Am Rev Respir Dis 131:690-695.). A local imbalance between proteinases and their physiological inhibitors results in pulmonary parenchyma damage by leakage of a protein-rich fluid into the interstitium and alveolar spaces. Major pulmonary proteinase inhibitors include α1-proteinase inhibitor (α1-PI), secretory leukocyte proteinase inhibitor (SLPI), and elafin (PI3) (Pfundt, R., et al. 1996. J Clin Invest 98:1389-1399; and Kramps, J. A., et al. 1991. Ann N Y Acad Sci 624:97-108.). Unlike high-molecular-weight α1-PI, which is mainly produced by the liver and reaches the lung via passive diffusion, SLPI and PI3 are low-molecular-weight inhibitors and are produced locally at neutrophil infiltration site in the lung (Sallenave, J. M., et al. 1994. Am J Respir Cell Mol Biol 11:733-741.). PI3 and SLPI are important antiproteinases in the lung in both health and disease (Tremblay, G. M., et al. 1996. Am J Respir Crit. Care Med 154:1092-1098.), and also demonstrate multiple biological functions, such as antibacterial activity, anti-inflammatory activity, priming of innate immunity, tissue remodeling and cellular differentiation, and augmentation of antiviral adaptive immunity Clin Sci (Lond) 110:21-35.).

Contrary to extensive molecular characterization of PI3, there are limited studies directly investigating the potential protective effects of PI3 in acute lung injury (ALI) and ARDS. In a murine model of lung injury mediated by Pseudomonas aeruginosa, intratracheal administration of human elafin encoded on an adenovirus vector showed significant protection against lung injury by reduced protein concentrations in BAL fluid, increased elimination of bacteria from the airways, and decreased incidence of hematogenous bacterial dissemination (Simpson, A. J., et al. 2001. J Immunol 167:1778-1786.). In another study by Tremblay et al., recombinant human pre-elafin exhibited a significant protective effect against human neutrophil elastase (HNE) induced acute lung injury in hamsters in a dose-dependent manner (Tremblay, G. M., Chest 121:582-588. 2002). In contrast to pre-elafin, elafin did not show such a protective effect within the same study. Elafin was the proteolytic product of pre-elafin, containing less transglutaminase substrate motifs (Guyot, N., et al. 2005. Biol Chem 386:391-399.). Both pre-elafin and elafin could be cross-linked to ECM protein, catalyzed by transglutaminase, and still exert anti-proteinase function (Guyot, N., et al. 2005. EBiochemistry 44:15610-15618.). However, additional transglutaminase substrate motifs contained in pre-elafin might allow stronger binding locally to inflammatory sites and show stronger protection effects than elafin.

Currently, there is only one report of measuring the BAL levels of PI3 and SLPI, as well as α1-PI, in human population (Sallenave, J. M., et al. 1999. Eur Respir J 13:1029-1036.). Although significant increases of PI3 were observed among ARDS subjects and at-risk subject without ARDS compared with healthy individuals, there was no significant difference of PI3 between ARDS subjects and at-risk subject without ARDS. In contrast, both SLPI and α1-PI demonstrated significant increases between two subject groups. Furthermore, almost all of the detectable PI3 was associated with high-molecular-weight proteins as revealed by western blot analysis.

### Example 2

### Polymorphisms in Neutrophil Elastase Inhibitor (elafin/PI3) are Associated with Risk of Acute Respiratory Distress Syndrome

### Method

The entire genomic sequence of PI3 gene on chromosome 20 (20q12-q13), ranging from ˜2,200 by upstream to ˜1760 bp downstream of the translation start site, in 28 anonymous DNA samples from healthy Caucasians of similar age and living in the same region of the parent study. Twenty-four polymorphisms were identified, including 21 SNP and 3 ins/del polymorphisms. Ten of them are novel polymorphisms in the studied population. Similar to a previous report (Chowdhury et al. BMC Med Genet, 2006. 7: p. 49), the analyses discussed herein describe many polymorphisms (n=9) located in the promoter region of PI3 genes with potential differential binding for at least one transcription factor. In addition, two common non-synonymous substitutions in exon 1 and exon 2 were also identified in our population. Linkage-disequilibrium (LD) structure analysis of 21 SNPs revealed two LD blocks with complete linkage disequilibrium. The first LD block contained 11 SNPs located in a region containing the promoter, exon1 and intron 1, and the other LD block had 9 SNPs from exon 2 to the 3′-UTR.

SNP A959C (rs2664581) with a nonsynonymous substitution of T34P within the first transglutaminase substrate domain, as well as two tagSNPs (A162T, rs1983649; A751T, rs6032040) in linkage disequilibrium with A959C (D's: 0.95-1.0), were genotyped (TaqMan) in 444 ARDS patients and 1133 critical ill patients from the ongoing Molecular Epidemiology of ARDS at the Massachusetts General Hospital and Harvard School of Public Health, Boston, Mass.

### Results

The three polymorphisms were in Hardy-Weinberg disequilibrium (p>0.46 by χ² goodness of fit). The A959C (T34P) polymorphism was significantly associated with increased risk of ARDS (crude OR(Odds Ratio)=1.38, 95% Confidence Interval (CI), 1.13-1.67; P_{trend}=0.0012; adjusted OR=1.69, 95% CI, 1.14-2.49, P_{trend}=0.0089). Stronger associations were observed in subjects aged <60 years (Adjusted OR=2.09, 95% CI, 1.21-3.61, P_{trend}=0.008). In haplotype analysis, the TTC (162T-751T-959C) haplotype was associated with higher risk of ARDS, with crude OR=1.39, (95% CI, 1.11-1.66, p=0.0023), and adjusted OR=1.39(95% CI, 1.11-1.73, p=0.0035), particularly in subjects aged <60 years (crude OR=1.62, 95% CI, 1.15-2.29, p=0.0057; adjusted OR=1.70, 95% CI, 1.18-2.44, P=0.0037), when compared with the most common ATA haplotype.

In conclusion, finding one or more of these polymorphisms in subjects at risk for developing ARDS indicates that the subject is more likely to develop ARDS than at-risk subjects who do not have the polymorphisms.

### Example 3

### Plasma Levels of PI3, SLPI and HNE in ARDS and At-Risk Controls

PI3 (elafin) and secretory leukocyte proteinase inhibitor (SLPI) are important low-molecular-weight proteinase inhibitors produced locally at neutrophil infiltration site in the lung (Schalkwijk, J., O. Wiedow, and S. Hirose, Biochem J, 1999. 340 (Pt 3): p. 569-77; Sallenave, J. M., et al., Am J Respir Cell Mol Biol, 1994. 11(6): p. 733-41; Kramps, J. A., et al., Ann N Y Acad Sci, 1991. 624: p. 97-108; Pfundt, R., et al., J Clin Invest, 1996. 98(6): p. 1389-99; Tremblay, G. M., et al., Am J Respir Crit Care Med, 1996. 154(4 Pt 1): p. 1092-8). In contrast to SLPI, PI3 (elafin) has a narrow spectrum of inhibition specifically toward human neutrophil elastase (HNE) and proteinases 3, mainly released by activated neutrophils, which play a crucial role in the initiation and propagation of ARDS (Weiland, J. E., et al., Am Rev Respir Dis, 1986. 133(2): p. 218-25). Considerable evidence exists for the role of neutrophil-derived proteinases in the pathogenesis of ARDS, including neutrophil elastase and collagenase (Christner, P., et al., Am Rev Respir Dis, 1985. 131(5): p. 690-5; Kawabata, K., T. Hagio, and S. Matsuoka, Eur J Pharmacol, 2002. 451(1): p. 1-10; Kodama, T., et al., Intern Med, 2007. 46(11): p. 699-704; Fujishima, S., et al., Biomed Pharmacother, 2007; Lee, C. T., et al., N Engl J Med, 1981. 304(4): p. 192-6; McGuire, W. W., et al., J Clin Invest, 1982. 69(3): p. 543-53).

A local imbalance between proteinases and their physiological inhibitors results in pulmonary parenchyma damage by leakage of a protein-rich fluid into the interstitium and alveolar spaces. The results of Example 1, revealed a differential expression of PI3 gene associated with ARDS development, however, HNE expression was only observed in a few blood samples while the majority of samples did not express this gene in the same study. This observation is consistent with previous findings that the expression of HNE gene is tightly controlled at the promyelocyte stage in bone marrow, but not expressed in mature neutrophils (Fouret, P., et al., J Exp Med, 1989. 169(3): p. 833-45; Yoshimura, K. and R. G. Crystal, Blood, 1992. 79(10): p. 2733-40).

Therefore, the protein expression levels of HNE, as well as PI3 and SLPI, in plasma samples from 147 ARDS cases, 63 at-risk controls and 28 healthy individuals were investigated using ELISA assays. Comparing to healthy individual, significantly elevated plasma levels of HNE, PI3 and SLPI were found in both ARDS cases and at-risk controls. Similar to the findings in the previous examples, there was a significantly elevated PI3 level before the onset of ARDS, followed by a trend of decreasing plasma PI3 levels from pre-diagnosis group to post-diagnosis group. The pre-diagnosis group had the highest plasma PI3 level, which was statistically significantly higher than the day of diagnosis group (P=0.004) and post-diagnosis group (P=0.0005). In contrast, both SLPI and HNE plasma levels decreased from pre-diagnosis group to post-diagnosis group but not significantly. However, when compared with plasma levels on the day of ICU admission of at-risk controls, pre-diagnosis ARDS group had significant higher PI3 and HNE, but not SPLI (P values: 0.026, 0.038, and 0.778, respectively). These findings showed that, along with the early progress of ARDS, plasma HNE maintained at high level was accompanied by continuous loss of anti-elastase capacity (PI3).

The ratio of PI3/HNE was then examined. Initial analyses revealed this ratio were highly significantly lower in samples with the onset of ARDS after adjustment of age, gender, APACHE III score at the day of ICU admission, and type of lung injury (direct vs. indirect). In addition, in the at-risk controls, a statistically significant decrease of plasma HNE levels (P=0.0015), but not PI3 (P=0.335), was found three days after the day of ICU admission, with a significant increase of the PI3/HNE ratio (P=0.0028). These results demonstrated that the loss of balance between elastase and its inhibitor, represented by the ratio of PI3/HNE, can be used as a biomarker for ARDS development. In particular, the clinical progress of ARDS may be monitored by measuring the amount of HNE, elafin, and/or the PI3/HNE ratio at several time points within the same subject or by comparing measurements in a subject with control samples.

### Introduction to Examples 4-8

An exploratory study was conducted of genome-wide gene expression in whole blood and found that the expression of neutrophil elastase inhibitor (PI3, elafin) was down-regulated during the early phase of ARDS. Further analyses of plasma PI3 levels revealed a rapid decrease during early ARDS development. PI3 and secretory leukocyte proteinase inhibitor (SLPI) are important low-molecular-weight proteinase inhibitors produced locally at neutrophil infiltration site in the lung. It was hypothesized and then demonstrated that plasma changes of HNE and its inhibitors can be used as surrogate markers in monitoring the clinical progress of ARDS. Therefore, a comprehensive investigation of plasma profiles of elafin, SLPI, and HNE in ARDS patients and as-risk controls was conducted. Here it is shown that the break of the balance between HNE and PI3 toward excess HNE is correlated with ARDS development. In these studies, in was demonstrated that an imbalance between neutrophil elastase (HNE) and its inhibitors in blood is related to the development of ARDS. PI3, SLPI, and HNE were measured in plasma samples collected from 148 ARDS patients and 63 critical ill patients at risk for ARDS (controls). Compared with the controls, the ARDS patients had higher HNE, but lower PI3, at the onset of ARDS, resulting in increased HNE/PI3 ratio (mean=14.5; 95% Cl, 10.9-19.4, P<0.0001). Although the controls had elevated plasma PI3 and HNE, their HNE/PI3 ratio (mean=6.5; 95% CI, 4.9-8.8) was not significantly different from the healthy individuals (mean=3.9; 95% CI, 2.7-5.9). Before the onset (7-days period prior to ARDS diagnosis), significantly elevated HNE was observed, but the HNE-PI3 balance remained normal. With the progress of acute lung injury from prior to the onset of ARDS, the plasma level of PI3 declined, whereas HNE was maintained at a higher level, tilting the balance toward more neutrophil elastase in the circulation as characterized by an increased HNE/PI3 ratio. In contrast, three days after ICU admission, there was a significant drop of HNE/PI3 ratio in the at-risk controls. Finally, plasma SLPI was not associated with the risk of ARDS development. The plasma profiles of PI3, HNE, and HNE/PI3 are useful clinical biomarkers in monitoring the development of ARDS and the efficacy of treatment. In one embodiment the plasma HNE level can be used to predict onset of ARDS. In another embodiment the ratio of HNE to elafin in blood or blood plasma can be used to predict ARDS or employed as an indicator of progression of lung injury toward ARDS. In such an embodiment a larger HNE/elafin ratio indicates progression of the disease toward ARDS.

### Example 4

### The Study Population, Plasma Collection, and Analysis

This study was conducted within the ongoing Molecular Epidemiology of ARDS project at the Massachusetts General Hospital (MGH) in Boston, Mass., which is a prospectively-enrolled cohort study of ARDS. All patients enrolled from adult intensive care units (ICU) at MGH were at risk for the development of ARDS with well-characterized predisposing clinical conditions, and were followed prospectively for the development of ARDS during their ICU stay (Gong, M. N., et al. 2005. Eur Respir J26:382-389; Gong, M. N., et al. 2004. Chest 125:203-211.). Predisposing clinical conditions are: 1) sepsis, 2) septic shock, 3) trauma, 4) pneumonia, 5) aspiration, or 6) massive transfusion of packed red blood cells (PRBC: defined as greater than 8 units of PRBC during the 24 hours prior to admission) as previously described (Gong, M. N., et al. 2005. Crit Care Med 33:1191-1198.). ARDS cases were defined by the American European Consensus Committee (AECC) criteria (Bernard, G. R., et al. 1994. Am J Respir Cult Care Med 149:818-824.). Controls were identified as at-risk patients who did not meet criteria for ARDS during their stay in the ICU and had no prior history of ARDS. Baseline clinical information, vital signs, and laboratory testing results in the first 24 hours of ICU admission were collected for calculation of the Acute Physiological and Chronic Health Evaluation (APACHE) III score (Knaus, W. A., et al. 1991. Chest 100:1619-1636.). In addition to a blood collection for DNA extraction during ICU stay, plasma samples were also collected for long-term storage. Based on the original protocol, two plasma samples were collected from each recruited subject, with the first sample collected during the first 24 hours of ICU admission and the second sample collected three days after the first collection. The study was approved by the Human Subjects Committee of MGH. A written informed consent was obtained from each subject or an appropriate proxy of the patient.

Of 1,686 enrolled patients who had ARDS risk factors and no exclusion criteria in this study, 509 patients eventually developed ARDS during ICU hospitalization. One hundred and forty-eight ARDS patients provided 194 plasma samples. When these ARDS patients who provided plasma samples were compared to those ARDS patients in whom plasma samples could not be obtained, there were no significant differences with regard to age, gender, ethnicity, or other relevant baseline characteristics (P>0.05 for all comparisons). However, the studied ARDS cases had significantly higher proportions of pneumonia, direct lung injury, and pre-admission steroid use, as well as higher APACHE III score on ICU admission. Sixty-three critically ill patients with ARDS risk factors randomly selected as controls had baseline characteristics that were not significantly different from the unselected controls (P>0.05 for all comparisons) except that selected controls had a higher APACHE III score on ICU admission (P=0.03). Baseline characteristics between selected ARDS cases and controls of this study, as shown in Table 7, were similar to those published in previous studies using the same study population (Gong, M. N., et al. 2005. Crit Care Med 33:1191-1198; Zhai, R., et al. 2007. Crit Care Med. 35:893-898.).

### Plasma Sample Collection

Based on the original sample protocol, plasma was collected from each enrolled patient within 48 hours of ICU admission, and a second sample was collected three days later. If an enrolled patient developed ARDS, two additional samples were collected corresponding to the first 48-hour of ARDS diagnosis and three days later. However, given the critical condition of ICU patients, difficulties in identifying surrogates and obtaining consent in time, and the limitation of total blood drawn from each subject set by the IRB, only 194 plasma samples could be collected from 148 ARDS cases. Since over 90% of ARDS cases developed ARDS during the first 7 days of ICU admission, the ARDS plasma could be divided into three groups, the pre-ARDS samples (up to 7 days before ARDS diagnosis, n=19), the ARDS samples (within 48 hours of diagnosis, n=67), and the post-ARDS samples (day 2 to 4 of diagnosis, n=105). In the current study, all available samples were used except three samples collected more than 8 days before ARDS diagnosis. Also, 63 pairs of plasma from the controls without ARDS were selected randomly, which were within 48 hours of ICU admission and three days later after the first collection. Furthermore, 28 anonymous plasma samples collected from male healthy individuals were included as reference samples.

### ELISA Analysis of Plasma Profile

Plasma samples were stored at −80° C. until analysis. Plasma PI3, SLPI, and HNE levels were quantified in duplicate using Human pre-ELAFIN/SKALP (Cat. No. HK318), Human SLPI (Cat No. HK316), and Human Elastase (Cat No. HK319) ELISA Test Kit from Cell Sciences (Canton, Mass.), according to the manufacturer's recommended protocol. Eleven plasma samples were randomly selected as replicates for quality evaluation.

### Statistical Analysis

The baseline characteristics between groups were compared using chi-square test for categorical variables, and two sample t-tests for continuous variables. Since plasma profiles of PI3, SLPI, HNE, and HNE/PI3 ratio suggested that they were not normal but from skewed distributions, the natural log transformation was applied to the data to adjust normality in the analyses. Plasma profiles among different sample groups were compared by two sample t-test, ANOVA (general linear model), and mixed effect model for repeated samples. Paired t-test and Wilcoxon sign-rank test were used as well to analyze the paired samples. In ANOVA analyses, Bonferroni correction was used to adjust for multiple comparisons. The relationship between ARDS plasma profiles and the sampling date relative to ARDS diagnosis was investigated by mixed effect models.

Clinical relevant covariates were adjusted in the analyses, including age, gender, type of lung injury, pre-admission steroid use, septic shock, and APACHE III score on ICU admission. Patients with pneumonia, aspiration, pulmonary contusions, or sepsis from lower pulmonary source were categorized as direct lung injury; whereas, patients with sepsis from an extrapulmonary source, trauma without pulmonary contusions, and multiple transfusions were categorized as indirect lung injury. Patients with both direct and indirect lung injuries were considered to have direct lung injury. All statistical analyses were performed by using the SAS statistical software package (version 9.1, SAS Inc., Cary, N.C.).

### Example 5

### Correlations Among Plasma Profiles in the ARDS and the Control

Plasma profiles of PI3, SLPI, and HNE on 59 ARDS plasma (collected within 48-hour of diagnosis) and were compared to 63 control samples (collected within 48-hour of ICU admission). Plasma PI3 correlated with plasma SLPI in the ARDS samples (Pearson correlation coefficient: ρ=0.36, P=0.003) and in the control samples where the correlation was stronger (ρ=0.50, P<0.0001). In contrast, plasma PI3 had a moderate correlation with plasma HNE in the ARDS sample (ρ=0.42, P=0.0004), but not in the control sample (ρ=0.03, P=0.815). In both the ARDS and the control, plasma PI3 and SLPI demonstrated moderate correlations with APACHE III score on ICU admission (ρ=0.37-0.53, P<0.005 for all analyses), but plasma HNE only showed correlation with APACHE III score in the ARDS samples (ρ=0.36, P=0.003). In addition, it was found that age was correlated with plasma PI3 (ρ=0.42, P=0.0005) and SLPI ρ=0.28, P=0.02) only in the controls but not in the ARDS sample (P=0.353 and P=0.291 for PI3 and SLPI, respectively), whereas, age was correlated with plasma HNE only in the ARDS sample (ρ=−0.30, P=0.015) but not in the controls (P=0.336). Since plasma PI3 and HNE showed some different correlations between the ARDS samples and the control samples, the combination of the two variables, by calculating the ratio of HNE/PI3, was included in the subsequent analyses.

### Example 6

### Comparison of Plasma Profiles Between ARDS and Controls

As shown in Table 8, both the ARDS samples (n=67, 48-hour after diagnosis) and the ICU control samples (n=63, 48-hour of ICU admission) had significantly elevated plasma levels of PI3, SLPI, and HNE (P<10−6 for all comparisons, two sample t-tests), as compared with the reference plasma samples from healthy individuals (n=28). When compared with the controls on ICU admission in two sample t-tests, there were significantly higher levels of plasma HNE (P=0.007) and HNE/PI3 ratio (P=0.001) at ARDS diagnosis, but no significant difference in plasma SLPI (P=0.657). The ARDS samples had a lower plasma PI3 but did not show statistical significance (P=0.115). After adjusting for age, gender, type of lung injury, septic shock, pre-admission steroid use, and APACHE III score on ICU admission, there was a significantly lower level of plasma PI3 in the ARDS, whereas, the results of the rest tests were unchanged (Table 8).

The opposite changes of PI3 and HNE between the ARDS and the control, which resulted in a highly increased HNE/PI3 ratio, showed that the balance between neutrophil elastase and its inhibitor was severely damaged with elastase in dominant in blood at the onset of ARDS. In contrast, the HNE/PI3 ratio was not statistically significant different between the controls and the reference samples (two sample t-test, P=0.105). In subgroup analyses within different types of lung injury, similar significant changes in plasma profiles of PI3 and HNE in the direct lung injury group were found, but not in the indirect lung injury group (Table 8). Moreover, either in the ARDS or in the controls, no significant difference was observed in plasma profiles between different types of lung injury.

Accordingly, in some embodiments the HNE/PI3 ratio can be used to predict ARDS. In a preferred embodiment, the HNE/PI3 ratio can be used to predict ARDS (or progression toward ARDS) in subjects with direct lung injury.

### Example 7

### Plasma Profiles Among Pre-ARDS, ARDS and Controls

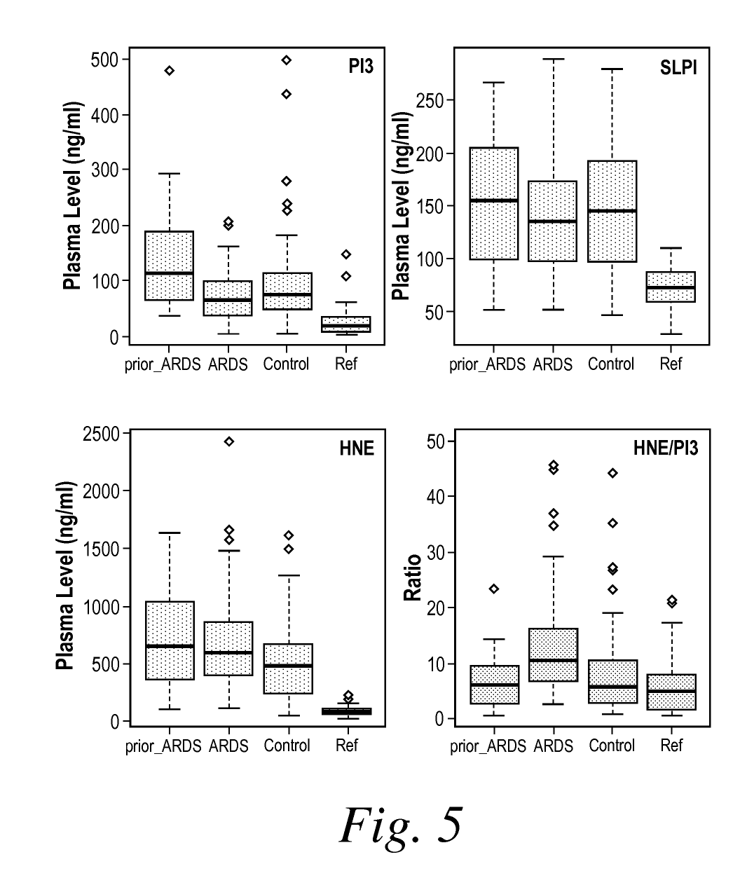

To explore the usefulness of using plasma profiles to predict the risk of ARDS development, samples collected on ICU admission were examined. Only a fraction of ARDS patients were diagnosed during the first 48-hour of ICU admission, whereas, the rest were diagnosed later during the ICU stay. Thus, samples could be classified into three groups, including pre-ARDS (n=7), ARDS (n=21) and control (n=63). Considering relatively small number of samples in the pre-ARDS group, all available pre-ARDS samples (n=19) and ARDS (within 48-hour diagnosis, n=67) were combined in the analyses. The difference in levels of plasma PI3, HNE and HNE/PI3 were statistically significant among the three groups in both crude analyses (ANOVA, P=0.017, 0.020, and 0.005, respectively), and in the adjusted analyses with age, gender, direct lung injury, preadmission steroid use, and higher APACHE III score on ICU admission (Generalized linear model, P=0.009, 0.017, and 0.0001, respectively). In pairwise comparisons with Bonferroni correction, firstly, similar changes of plasma profiles were found between the ARDS and the controls, with plasma HNE and HNE/PI3 ratio significantly higher in the ARDS. However, PI3 did not reach significance after correction for multiple comparisons (FIG. 5). Secondly, when comparing the pre-ARDS and the ARDS, the ARDS group had significantly lower plasma PI3 but higher HNE/PI3 ratio than the pre-ARDS group.

However, there was no significant difference in HNE between the pre-ARDS and the ARDS samples. Finally, the plasma HNE in pre-ARDS was significantly higher than the controls (P=0.018), but no significant difference of plasma PI3, SLPI, and HNE/PI3 ratio was observed between the two groups (Table 9). Accordingly, plasma HNE levels may be used to differentiate subjects who have pre-ARDS from those who are merely at risk for developing the disease. Furthermore, although the pre-ARDS had significantly higher PI3, SLP, and HNE than the reference samples (P<10−6 for all comparisons, two sample t-tests), the HNE/PI3 ratio was not significantly different between the two groups (P=0.276, two sample t-tests). In summary, these findings indicated that the balance between PI3 and HNE was maintained before the onset of ARDS.

### Example 8

### Decrease of Plasma PI3 with the Clinical Progress of ARDS

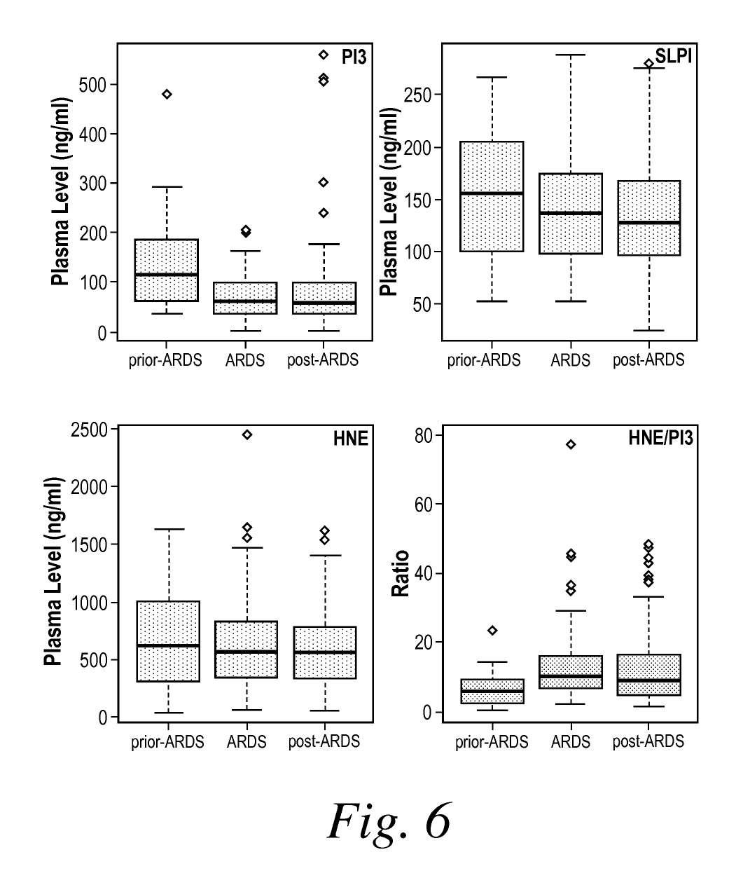

Our previous results showed that reduction of PI3 along with the clinical progress of ARDS was the cause for breaking the HNE-PI3 balance. Next, the changes of plasma profiles were examined among three sample groups from ARDS patients, including pre-ARDS, at ARDS diagnosis, and post-ARDS diagnosis. The PI3 and HNE/PI3 were significantly changed among three groups in both the crude analyses (ANOVA, P=0.004 and P=0.010, respectively), and in the analyses adjusted for age, gender, direct lung injury, pre-admission steroid use, and APACHE III score on ICU admission (Generalized linear model, P=0.015 and P=0.036, respectively). The pre-ARDS samples had significantly higher PI3, but lower HNE/PI3, than either the ARDS or the post-ARDS in pairwise comparisons after correction for multiple comparisons (FIG. 6). In contrast, there was no difference in plasma SLPI and HNE among three samples groups. The trend of plasma PI3 decrease across ARDS development, as measured by sampling dates relative to ARDS diagnosis, was further tested using mixed effect models.

The reciprocal of days between sampling dates relative to ARDS diagnosis was significantly related to plasma PI3 and HNE/PI3 (mixed models, P=0.0008 and P=0.002, respectively), showed that the closer to ARDS onset, the greater the decrease in PI3 and the higher degree of HNE/PI3 imbalance.

### Example 9

### Changes of Plasma Profiles Between Paired Samples

The changes in plasma profiles in patients who did not develop ARDS during ICU stay were investigated between paired samples collected within 48 hours of ICU admission and three days after the first sample collection (Table 10). As compared with the ICU admission, the second plasma had significantly lower HNE (paired t-test, P=0.003; Wilcoxon sign-rank test, P=0.0008), accompanied by a lower HNE/PI3 ratio (paired t-test, P=0.001; Wilcoxon sign-rank test, P=0.0004). This observation demonstrated that the elevated plasma HNE was decreased in those patients who did not develop ARDS. Interestingly, it was found the plasma profiles changed differently by type of lung injury, while the final results were in favor of maintaining enough inhibition capacity against HNE. In the patients with direct lung injury, there was a significant decrease of HNE (P=0.023) without significant changes in PI3 (P=0.302). Whereas, in the patients with indirect lung injury, there was a significant increase of PI3 (P=0.023) without significant changes in HNE (P=0.069). Twenty-four ARDS patients provided paired plasma in the study, with the first sample collected during the first 48 hours of ARDS diagnosis and second sample collected three days later. However, there was no significant change in PI3 (P=0.131), SLPI (P=0.226), HNE (P=0.552), and HNE/PI3 (P=0.083) between two time points.

### Discussion of Examples 4-9

Previous research showed that a local imbalance between proteinases and their physiological inhibitors results in pulmonary parenchyma damage by leakage of a protein-rich fluid into the interstitium and alveolar spaces, a process that plays a crucial role in the initiation and propagation of ARDS (Kawabata, K., et al. Eur J Pharmacol. 2002. 451:1-10). The examples described herein extend this observation into the peripheral circulation. At the onset of ARDS, the ARDS patients had significantly higher HNE but lower PI3 resulting in increased HNE/PI3 ratio (mean=14.5; 95% CI, 10.9-19.4), compared with patients who did not develop ARDS during the ICU stay which had a higher but not statistically significant HNE/PI3 ratio (mean=6.5; 95% CI, 4.9-8.8) than that of healthy individuals (reference samples: mean=3.9; 95% CI, 2.7-5.9). Before the ARDS onset (7-day period prior to ARDS diagnosis), it was observed that only a significantly elevated level of HNE was present but the HNE/PI3 ratio was unchanged as compared with either the at-risk controls at the ICU admission or the healthy reference group. With the progress of acute lung injury from prior to the onset of ARDS, the PI3 plasma level was dropped whereas HNE was maintained at a higher level, tilting the balance toward more neutrophil elastase in the circulation characterized by increased HNE/PI3 ratio. In contrast, three days after ICU admission, there was a significant drop of the HNE/PI3 ratio in the at-risk controls. Therefore, these results demonstrate that the imbalance between plasma neutrophil elastase (HNE) and its specific inhibitor elafin (PI3) is related to the risk of ARDS development. Furthermore, the plasma profiles of PI3, HNE, and HNE/PI3 can be used as clinical biomarkers in monitoring the development of ARDS.

Nevertheless, plasma SLPI was not associated with the risk of ARDS development. Although both PI3 and SLPI are low-molecular weight proteinase inhibitors belonging to the same chelonianin family of canonical inhibitors (Family 117 Clan IP in the MEROPS database), PI3 has a much narrower anti-proteinase spectrum since it inhibits only neutrophil elastases and proteinase 3 (Moreau, et al. Biochimie. 2008. 90:284-295.). In addition, PI3 has a unique N-terminal non-inhibitory domain, containing several transglutaminase substrate motifs, which can covalently anchor the whole molecule to extracellular matrix (ECM) protein through protein cross-linking (Nara, K., et al. J Biochem (Tokyo). 1994. 115:441-448; Guyot, N., et al. Biochemistry. 2005. 44:15610-15618). Small molecular size, specific anti-elastase spectrum, and immobilized to ECM are major characters determine the PI3 plays critical protective roles against the tissue damage induced by HNE during acute lung injury. The expression of PI3 gene can be readily induced under inflammatory conditions by proinflammatory cytokines, such as IL-β1 and TNF-α, and elastase (Sallenave, J. M., et al. 1994. Am J Respir Cell Mol Biol 11:733-741; Pfundt, R., et al. 2000. Arch Dermatol Res 292:180-187; Reid, P. T., et al. 1999. FEBS Lett 457:33-37.). Other data described herein revealed that PI3 gene demonstrated the largest down-regulation in peripheral blood expression at the early stage of ARDS, as compared with the recovery stage around ICU discharge (Wang, Z., et al. 2005. Environ Health Perspect 113:233-241.). In a smaller set of samples (40 ARDS patients and 23 controls), the protein expression in plasma was correlated well with the microarray findings with a lower level of plasma PI3 during the acute-stage. In the same study, it was shown that there was a trend of plasma PI3 decreasing from pre- to post-diagnosis of ARDS. The results of this study confirmed the previous study by observing the same PI3 decreasing trend along with clinical progress toward ARDS development, with a larger sample size within the same study population (148 ARDS patients and 63 controls). Additional analyses of plasma PI3 were carried out by pooling data from both studies, which had overlapped samples collected from 24 ARDS patients and 13 at-risk controls, and obtained consistent results (data not shown).

At the end of proteinase-inhibitor balance is the HNE, which has been implicated in the pathogenesis of ALI/ARDS in previous studies (Abraham, E. 2003. Crit Care Med 31:S195-199.). Elevated HNE was observed in animal models of ALI. Administration of HNE could induce typical symptoms of ALI in experiment animals, and inhibition of increased HNE could reduce ALI symptoms (Kawabata, K., et al. Eur J Pharmacol. 2002. 451:1-10; Zeiher, B. G., et al. 2002. Crit Care Med 30:S281-287.). Despite earlier clinical studies revealed conflict results of elevated HNE in bronchoalveolar lavage fluid (Weiland, J. E., et al. Am Rev Respir Dis. 1986 133:218-225; Lee, C. T., et al. 1981.N Engl J Med 304:192-196; Idel l, S., et al. 1985. Am Rev Respir Dis 132:1098-1105), more recent studies measuring plasma HNE found consistently increased plasma HNE in ALI/ARDS patients (Donnelly, S. C., et al. 1995. Am J Respir Crit Care Med 151:1428-1433; Rocker, G M., et al. 1989. Lancet 1:120-123; Fujishima, et al. 2007. Biomed Pharmacother; Gando, S. et al, 2004. Inflammation 28:237-244).

Here observations of significantly higher plasma HNE in the ARDS patients were consistent with the previous research. In one particular study examining sequential blood samples, Kodama et al. reported that the plasma HNE level of ALI/ARDS patients (n=18) with inflammatory response syndrome (SIRS) was significantly greater than that of SIRS alone (n=5) (Kawabata, K., et al. Eur J Pharmacol. 2002. 451:1-10). A cut off value of >220 ng/ml was also proposed in the same study, as HNE in all patients with SIRS alone was consistently less than this value and all SIRS patients with HNE more than 220 ng/ml eventually developed ALI/ARDS. Because of a small sample size, that study pooled ALI and ARDS together as the case group to compare with a patient group having less severe conditions. The findings described here that HNE levels are significantly elevated before the onset of ARDS (the pre-ARDS) agrees with a previous report that the onset of pulmonary dysfunction followed HNE increase.

Without being bound to any one theory, one possible mechanism is that, with the progress of pulmonary inflammation, the increased permeability allows free passing of HNE and PI3 through the pulmonary bloodgas barrier. It is more likely that PI3 has a net unidirectional flow from blood to lung parenchyma as PI3 will be trapped in ECM by transglutaminase-catalyzed cross-links. Elevated circulating PI3 acts as an extra-pulmonary resource for protecting unregulated proteolysis. When plasma PI3 drops to certain level, HNE-PI3 balance cannot be maintained in both circulation and lung with the occurrence of ARDS. This hypothesis may explain why a large multicenter clinical trial of sivelestat, a specific elastase inhibitor, failed to improve the survival rate of ALI/ARDS (Zeiher, B. G., et al. 2002. Crit Care Med 30:S281-287.). In that study, all patients were enrolled after the onset of ALI, which could be too late for the action of an elastase inhibitor. In another smaller clinical trial, sivelestat was administrated from the ICU admission resulting in significant reduction of mortality in critically ill patients (Hoshi, K., et al. 2005. Tohoku J Exp Med 207:143-148). On the other hand, our hypothesis also indicates that PI3 itself may be a drug candidate for specific ARDS treatment.

Although previous studies suggest that the circulating HNE could be used as a predictive factor for ALI/ARDS development, patients with predisposing risk conditions for ARDS often had increased plasma HNE with a large range of variation. Similarly, despite the fact that a significantly lower level of plasma PI3 in the ARDS patients was observed, there was a wide range of variation in both ARDS patients and the controls. PI3 and HNE are located at the opposite ends of the proteinase-inhibitor balance, and their expression levels could be affected by common as well as independent factors. Simultaneous measurement of PI3 and HNE will serve as a more reliable, composite marker to distinguish ARDS patients from those critically ill patients who did not develop ARDS. Since it is often impractical to assess local HNE-PI3 balance in lung, monitoring the plasma HNE-PI3 balance is a good alternative.

### Introduction to Examples 10-11

It was demonstrated that polymorphisms in PI3 gene is associated with the risk of developing ARDS. A case-control study was conducted in order to investigate whether genetic variants in the PI3 gene are associated with ARDS development. Based on resequencing data from 29 unrelated Caucasians, three tagging single nucleotide polymorphisms were selected and genotyped in a prospective cohort consisting of 449 ARDS Caucasian patients (cases) and 1031 critically ill patients (at-risk controls). The variant allele of rs2664581 (T34P) was significantly associated with increased ARDS risk (OR, 1.35; 95% CI, 1.09-1.67; P=0.006; FDR adjusted P=0.018). Since previous evidences suggested that the genetic association with ARDS might be modified by the origin of lung injury (Gong, M. N., et al. 2004. Chest, 125, 203-211; Gong, M. N., et al. 2005. Eur. Respir. J., 26, 382-389), a stratified analysis was conducted among patients with different types of lung injury (pulmonary vs. extrapulmonary lung injury). The association of the variant allele and ARDS was found to be stronger among subjects with extrapulmonary injury. The common haplotype Hap2 (TTC), containing the variant allele of rs2664581, was also identified as a risk haplotype for ARDS (OR, 1.31; 95% CI, 1.05-1.64; P=0.015). Furthermore, the rs2664581 polymorphism was associated with circulating PI3 levels in multivariate analyses. ARDS patients homozygous for the wild-type A allele of rs2664581 showed significantly lower PI3 plasma level (P=0.021) at ARDS onset as compared with those homozygous or heterozygous for the variant C allele.

The data described herein shows that polymorphisms in PI3 gene are significantly associated with ARDS risk and with circulating PI3 levels. Accordingly, in one embodiment of the invention a method of predicting whether a subject is at high risk of developing Acute Respiratory Distress Syndrome (ARDS) comprises determining whether a subject has the variant allele of rs2664581; wherein the presence of the allele indicates that the subject is at high risk of developing ARDS. In certain preferred embodiments, the subject has extrapulmonary injury. In another embodiment of the invention a method of predicting whether a subject is at high risk of developing Acute Respiratory Distress Syndrome (ARDS) comprises determining whether a subject has the haplotype Hap2 (TTC); wherein the presence of the haplotype indicates that the subject is at high risk of developing ARDS. In certain preferred embodiments, the subject has extrapulmonary injury.

### Materials and Methods for Examples 10-11

### Study Population

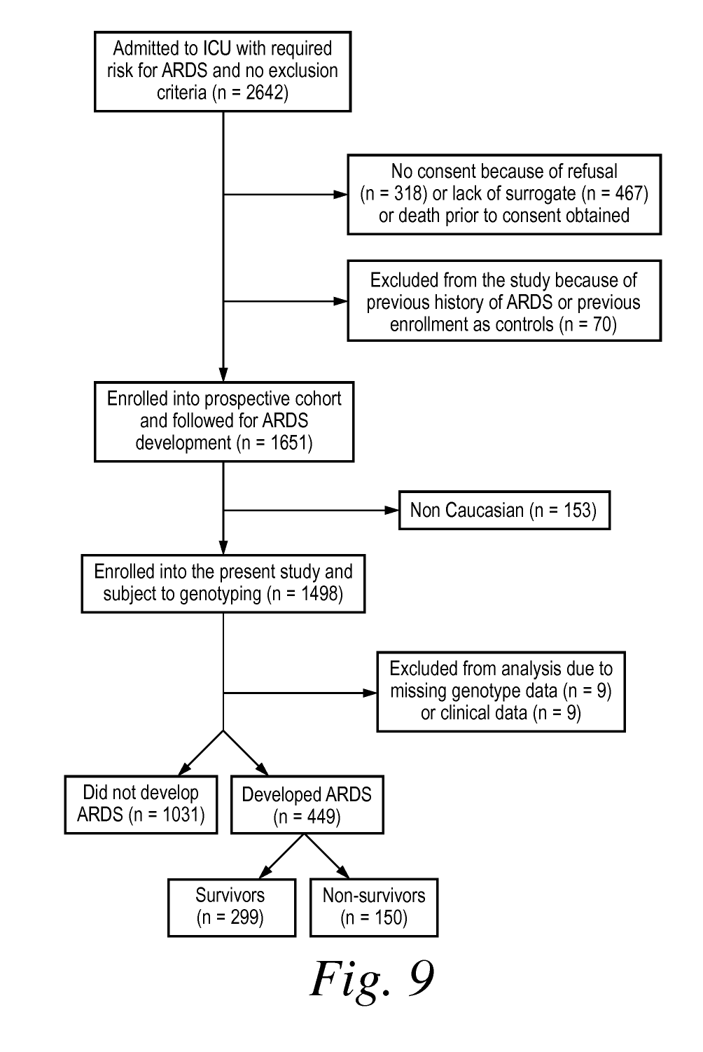

All subjects were recruited from patients admitted to the intensive care units (ICU) of the Massachusetts General Hospital (Boston, Mass.) between September 1999 and November 2006. Details of the recruitment have been described previously (Gong, M. N., et al. 2005. Crit. Care Med., 33, 1191-1198). Briefly, patients with pre-depositing conditions for ARDS development and without any of the exclusion criteria (Table 16) were enrolled and followed daily for the development of ARDS, as defined by the American-European Consensus Committee (AECC) criteria for ARDS (Bernard, G. R., et al. 1994. Am. J. Respir. Crit. Care Med., 149, 818-824). At-risk patients who did not meet the criteria for ARDS during the ICU stay were classified as controls. The flowchart of study design is illustrated in FIG. 9. Baseline characteristics were recorded on ICU admission and APACHE III scores were calculated based on the data collected within the first 24 hrs after ICU admission. The conditions of pneumonia, pulmonary contusion and aspiration were defined as pulmonary risk factors for ARDS (i.e., pulmonary injury), whereas sepsis and/or bacteremia originating from extrapulmonary sources, non pulmonary trauma and multiple transfusion were defined as extrapulmonary risk factors (i.e., extrapulmonary injury). All ARDS patients were followed for clinical outcomes of all-cause mortalities in 28 days and 60 days after ARDS diagnosis. To reduce possible interference from population stratification, all analyses were restricted to the non-Hispanic Caucasian subjects, which account for 90.7% of the studied population. This study was approved by the Human Subjects Committees of the Massachusetts General Hospital and the Harvard School of Public Health. An informed consent was obtained from each enrolled patient or an appropriate proxy.

During the study period a total of 1651 patients were enrolled into the prospective cohort. The final analyses were conducted in 1480 non-Hispanic Caucasians with complete clinical and genotyping data, including 449 ARDS cases and 1031 at-risk controls. The distribution of the baseline characteristics of the study population and the comparisons of co-morbidities and clinical risk factors for ARDS between groups are shown in Table 11. Subjects who developed ARDS were younger and have higher Acute Physiology Age and Chronic Health Evaluation (APACHE) III scores. Septic shock, pneumonia, pulmonary injury, liver cirrhosis/failure and history of alcohol abuse were more common in the ARDS group, whereas, sepsis without septic shock and diabetes were more common in the controls. No differences in gender distribution were observed between ARDS and controls. Baseline characteristics between ARDS cases and controls with available plasma samples were similar to those in the overall group (Table 17).

### Identification of Genetic Variations at PI3 Locus

The gene PI3 located in chromosome 20 (20q12-q13) is approximately 2.3 kb in length and is described as containing three exons and two introns (Sallenave, J. M. and Silva, A. 1993. Am. J. Respir. Cell Mol. Biol., 8, 439-445). 2 kb of the 5′ and 3′ untranslated regions and all exons and introns using DNA samples from 29 nonrelated, healthy Caucasian subjects recruited as controls from a lung cancer susceptibility study were resequenced. These participants were friends or spouses of lung cancer cases. This sample size provides a >99% detection rate to identify polymorphisms with MAF >5% (Kruglyak, L. and Nickerson, D. A. 2001. Nat. Genet., 27, 234-6). Bidirectional sequence analyses were conducted at the Harvard-Partners Center for Genetics and Genomics, Boston. GenBank accession no. AL049767.12 sequence was used as the reference sequence for defining the sequence variants. Linkage disequilibrium (LD) blocks of the PI3 gene were determined by SNPs with MAF ≧10%. SNP PI3-1 could not be genotyped over 50% of the subjects in resequencing, so it was also excluded from the LD block determination. Haploview (v3.32) software was used to determine the LD structure as described previously by Gabriel et al (Barrett, J. C., et al. 2005. Bioinformatics, 21, 263-265; Gabriel, S. B., et al. 2002. Science, 296, 2225-2229.). To efficiently tag the common variation across the gene, tSNPs were selected using the r2-based Tagger program (de Bakker, P. I., et al. 2005. Nat. Genet., 37, 1217-1223) implemented in Haploview software, with the aggressive mode and the Tagger thresholds of r2≧0.8 and LOD score >3.

### Genotyping

Genomic DNA was extracted from whole blood sample using Puregene DNA Isolation Kit (Gentra Systems, Minneapolis, Minn.) or the AutoPure LS workstation using the Autopure reagents (Qiagen, Valencia, Calif.). SNPs rs19836491, rs6032040 and rs2664581 were genotyped using TaqMan® SNP Genotyping Assay (Applied Biosystems, Foster City, Calif.). Primers and probes were ordered from Applied Biosystems (TaqMan Assay ID: C_{—}25619045_{—}10, C_{—}29073396_{—}10, and C_{—}11656453_{—}1_, respectively), and genotyping was performed using the ABI Prism® 7900HT Sequence Detection System (Applied Biosystems, Foster City, Calif.). Laboratory personnel were blinded to case-control status. To ensure the quality of genotyping data, 10% of randomly selected samples were interspersed in the plates as replicates and all genotyping results were reviewed by two investigators independently. Based on comparison of replicate genotypes, the genotyping error rate was <0.05% and the overall genotyping success rate was above 98%. Samples not yielding the genotypes of all SNPs were excluded from analysis.

### Statistical Analysis

All statistical analyses were performed using the SAS statistical software package (version 9.1, SAS, Cary, N.C.). A P-value <0.05 was considered to be statistically significant. The demographic variables between ARDS patients and controls were tested by Fisher's exact test for categorical variables and by Student's t-test for continuous variables. SAS/Genetics PROC ALLELE was used test to calculate the allele frequencies, to test the deviation from Hardy-Weinberg equilibrium (HWE), and to estimate the pairwise D′ and r2 values for LD. Haplotype frequencies and individual haplotypes were estimated based on unphased genotype data using the expectation maximization algorithm as implemented in SAS/Genetics. The associations between PI3 haplotypes and the risk of ARDS were analyzed using the expectation-substitution approach as implemented in the SAS macro HAPPY (Kraft, P., et al. 2005. Genet. Epidemiol., 28, 261-272; Zaykin, D. V., et al. 2002. Hum. Hered., 53, 79-91). This approach treats subject-specific expected haplotype indicators, calculated by an additive model, as observed covariates for regression models. Haplotypes with a frequency greater than 5% in the total population were considered as “common” and the most common haplotypes was used as the reference in the logistic regression models.

Multivariate logistic regression was used to estimate the genotype- and haplotype-specific odds ratio (OR) and 95% confidence interval (CI) for ARDS susceptibility. The genotype associations were analyzed in both additive and dominant models. To evaluate the associations of individual genotypes and haplotypes with the survivals in patients with ARDS, the Cox proportional hazard model was used to estimate the hazard ratio (HR) and 95% CI. Covariates were chosen based on the potential risk for ARDS development and mortality, including age, gender, APACHE III score, and risk factors for ARDS, comorbidities, and alcohol abuse. Global test for the association between haplotypes and ARDS risk and survival were carried out using the likelihood ratio test (LRT), comparing the models with the haplotypes to the models without. For statistically significant associations, adjusted P-values were calculated to correct for multiple comparisons, using the False Discovery Rate (FDR) procedure of Benjamini and Hochberg (Benjamini, Y., et al. 2001. Behav. Brain Res., 125, 279-284). Since plasma profiles of PI3 had skewed distributions, the natural log transformed data were used in the analyses. Plasma PI3 levels between ARDS cases and controls were compared using two sample t-test and general linear model.

### Example 10

### Genetic Variations at PI3 Locus and ARDS

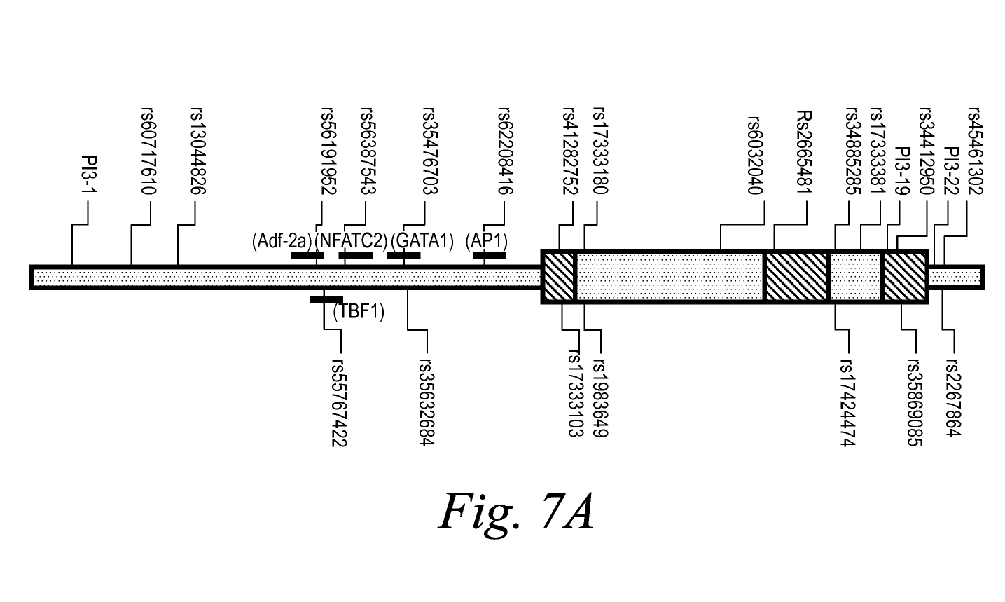

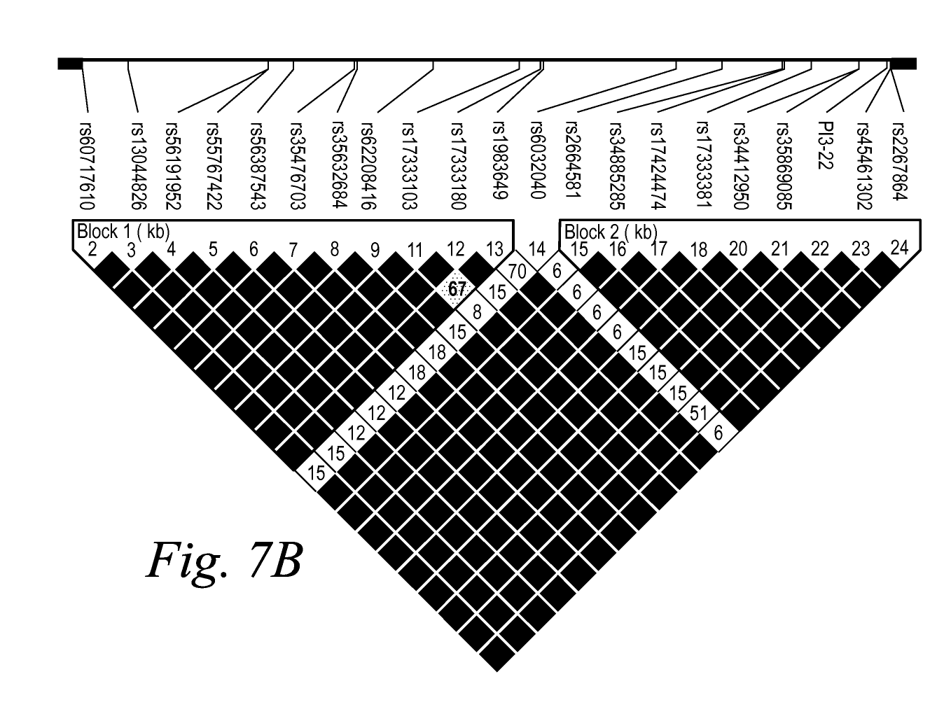

A total of 24 polymorphisms were identified at PI3 locus, including 21 SNPs and 3 deletion/insertion polymorphisms. Among them, three polymorphisms were novel variants that were neither published in dbSNP (ncbi.nlm.nih.gov/SNP), nor reported elsewhere. Positions and allelic frequencies of the identified SNPs are shown in Table 12. Nine polymorphisms were identified in the promoter region of PI3. Most of them have been predicted to have different binding sites for one or more transcription factors (Moraes, T. J., Chow, C. W. and Downey, G. P. 2003. Crit. Care Med., 31, S189-194). Three non-synonymous polymorphisms, resulting in amino acid substitutions and previously described in the dbSNP database, were found in the coding regions of the gene. In addition, there were two novel SNPs (PI3-19 and PI3-22) identified in the 3′-untranslated region. The genomic structure of PI3 and the relative physical position of the polymorphisms are presented in FIG. 7A.

Resequencing data was used to determine the LD structure of the PI3 gene (FIG. 7B). Two LD blocks were found that were constructed with SNPs rs60717610-rs1983649 and rs2664581-rs2267864. Each block showed limited haplotype diversity, with high coefficient between the two LD blocks (D′=1). Three tSNPs rs19836491, rs6032040, and rs2664581 (PI3-13, PI3-14, and PI3-15, respectively) were identified which capture all variants with minor allele frequency (MAF) ≧10% Genotype distributions of the selected tSNPs in the at-risk controls were in Hardy-Weinberg equilibrium. MAF for tSNPs between cases and controls are shown in Table 13. A significant difference was found in MAF of rs2664581 between the two groups (P=0.008, FDR adjusted P=0.024). A significant difference was also found in the genotype distribution of rs2664581 between ARDS patients and controls, but this difference was not statistically significant after adjustment for multiple comparisons (P=0.030, FDR adjusted P=0.090) (Table 18).

### Association Between PI3 Polymorphisms and the Risk of Developing ARDS

Table 14 summarizes the results of association tests assuming additive and dominant models. Under the additive model, the tSNPs rs2664581 was significantly associated with increased risk of developing ARDS in both crude (OR, 1.30; 95% CI, 1.07-1.58; P=0.008; FDR adjusted P=0.025) and adjusted analyses (OR, 1.35; 95% CI, 1.09-1.67; P=0.006; FDR adjusted P=0.018). In stratified analysis by source of lung injury, rs2664581 and rs1983649 were significantly associated with increased ARDS risk in patients with extrapulmonary injury, whereas no association with ARDS risk was found among patients with pulmonary injury. Similarly, when the association was tested using a dominant genetic model, rs2664581 was strongly associated with increased risk of developing ARDS (Table 14).

Eight inferred haplotypes were observed in the studied population. Four of them occurred at frequencies >5% and jointly accounted for a cumulative frequency of 99.8% in our population (Table 15). Significant differences were observed in the distribution of Hap2 (TTC) between ARDS and controls (P=0.009, FDR adjusted P=0.036). Haplotype-specific risk for ARDS was assessed using the most common haplotype (Hapl: ATA) as the reference in the regression model. The global haplotype association test demonstrated that PI3 haplotypes were marginally associated with ARDS risk (LRT, P=0.06). Consistent with the results of single polymorphism analysis, the haplotype (Hap2: TTC) containing variant alleles of rs2664581 and rs1983649, was significantly associated with increased ARDS risk (OR, 1.31; 95% CI, 1.05-1.64; P=0.015). In the stratified analysis, the same haplotype was associated with risk of ARDS among patients with extrapulmonary injury (OR, 1.67; 95% CI, 1.16-2.40; P=0.005), but the global test was not significant in this subgroup (LRT, P=0.096) (Table 15).

In the present study, 24 polymorphisms were identified, including 3 novel variants, in PI3 gene within the studied non-Hispanic Caucasian population. Three tSNPs (rs1983649, rs6032040, and rs2664581), which capture the entire common genetic variation in PI3 gene, were genotyped and analyzed for the association with ARDS. The variant allele of rs2664581 was significantly associated with increased ARDS risk. The haplotype (Hap2: TTC), which contains variant C allele of rs2664581, was also associated with increased risk for ARDS. These results show that genetic variations in the PI3 gene are associated with major susceptibility to ARDS.

Additionally, it was found that the genetic effects of PI3 polymorphisms on ARDS development were modified by the origin of lung injury. In stratified analysis, variants of rs 1983649 and rs2664581 were individually associated with increased ARDS risk in patients with extrapulmonary injury. Consistent with the SNP analysis, Hap2 containing the variant alleles of rs 1983649 and rs2664581, was also associated with ARDS development in patients with extrapulmonary injury. However, this association was not observed in patients with Hap4 carrying the wild type allele of rs2664581, suggesting that the association between rs1983649 and ARDS is driven by LD with rs2664581.

The modification of the genetic association with ARDS by the origin of lung injury has been observed previously. Recent studies in our cohort indicated that the −308G/A and LTA (TNFB) polymorphisms of the tumor necrosis factor genes were linked to ARDS in patients with pulmonary, but not extrapulmonary injury (Gong, M. N., et al. 2005. Eur. Respir. J., 26, 382-389). In the same cohort women with the variant SFTPB (SP-B) polymorphism of surfactant protein-B gene had an increased odd of having a pulmonary injury as a risk factor for ARDS (Gong, M. N., et al. 2004. Chest, 125, 203-211). These results were consistent with a recent report in which the −1580C/T missense mutation in the SP-B gene was associated to increased risk of ARDS in patients with pulmonary injury (Lin, Z., et al. 2000. Clin. Genet., 58, 181-191). It is known that ARDS derived from a pulmonary insult has different pathophysiological, biochemical, radiological and mechanical patterns from ARDS caused by an extrapulmonary injury (Pelosi, P., et al. 2003. Eur. Respir. J. Suppl., 42, 48s-56s).

In ARDS derived from a pulmonary insult (pulmonary ARDS) the prevalent damage in early stage is likely intra-alveolar, whereas in ARDS caused by extrapulmonary insult (extrapulmonary ARDS) the damage is focused on the vascular endothelium with a greater amount of inflammatory agents in the blood stream.

Recently, it has been also demonstrated that acute lung injury derived from different insults leads to different gene expression profiles. Genes stimulated by lipopolysaccharide in animal model from extrapulmonary acute lung injury were involved in metabolism, defense response, immune cell proliferation, differentiation and migration, and cell death, whereas genes involved in organogenesis, morphogenesis, cell cycle, proliferation, and differentiation were significantly expressed in response to high-volume ventilation. (dos Santos, C. C., et al. 2008. Crit. Care Med., 36, 855-865). Without being bound to any particular theory, the results indicate that genetic variants of rs2664581 may contribute to those processes involved in the pathogenesis of extrapulmonary ARDS. HNE has been shown to compromise the integrity of the endothelial vascular barrier and promote microvascular injury during ARDS development (Kawabata, K., et al. 2002. Eur. J. Pharmacol., 451, 1-10; Moraes, T. J., Chow, C. W. and Downey, G. P. 2003. Crit. Care Med., 31, S189-194).

### Association Between PI3 Polymorphisms and ARDS Survival

Baseline characteristics between survivors and non-survivors of ARDS patients were shown in Table 19. Among patients with ARDS, the survivors were younger and had lower APACHE III scores than the nonsurvivors. There were no significant differences in mortality rates at either 28-day or 60-day between survivors and nonsurvivors for any selected tSNP. None of the PI3 genotypes or haplotypes was significantly associated with 60-day survival in all subjects or in stratified subgroups (Table 20).

### Example 11PI3 Polymorphisms and Plasma PI3 Levels

The relationship of rs2664581 polymorphism and plasma PI3 level was investigated. In the parent study, in addition to blood samples for DNA extraction, plasma samples within 48 hours of ICU admission or ARDS diagnosis were also collected for long-term storage at −80° C. until analysis. Due to delay in contacting surrogates, death, and limitation of total blood drawn from some families, plasma within 48 hours of ARDS was available in 67 ARDS patients. About 55.2% ARDS patients developed ARDS within 48-hour ICU admission. Plasma collected within 48 hours of ICU admission was analyzed from 63 randomly selected controls. Plasma PI3 levels were quantified in duplicate using Human pre-ELAFIN/SKALP (Cat. No. HK318) ELISA Test Kit (Cell Sciences, Canton, Mass.), according to the manufacturer's protocol. Eleven plasma samples were randomly selected as replicates for quality evaluation.

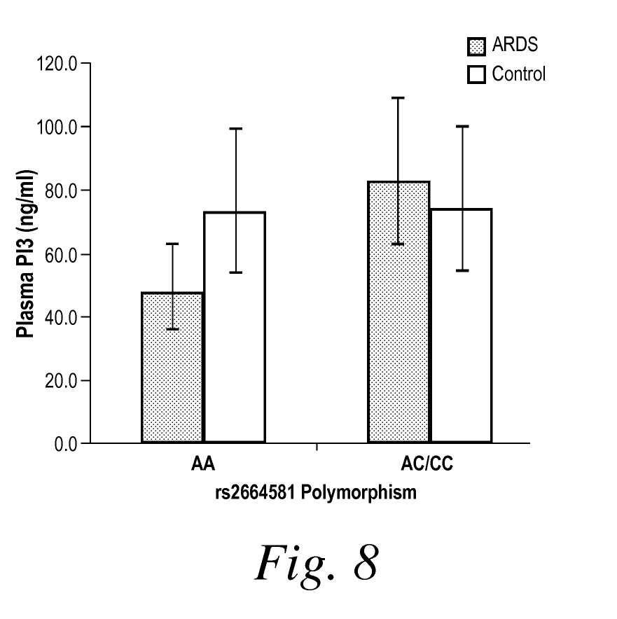

Genotyping failed in 4 ARDS patients and 2 controls with available plasma samples. ARDS patients with C allele (variant allele corresponding to P residue, n=21; adjusted mean=82.7 ng/ml; 95% CI: 62.9-108.9 ng/ml) showed significant higher level of plasma PI3 (P=0.021) at ARDS onset as compared with those with only A allele (wild-type allele; n=42; adjusted mean=47.7 ng/ml; 95% CI: 36.1-63.1 ng/ml) as shown in FIG. 8. In contrast, there was no difference between different alleles of rs2664581 in controls (C allele, n=21, adjusted mean=74.0 ng/ml; 95% CI: 54.7-100.1 ng/ml; A allele, n=40, adjusted mean=73.1 ng/ml; 95% CI: 53.7-99.4 ng/ml; P=0.083). Moreover, in the subgroup analysis of patients homozygous for A allele, the ARDS patients had significant lower plasma PI3 than the controls (P=0.007). On the contrary, among patients carrying variant allele, No difference was observed between plasma profiles between the ARDS and the controls. The results of further analyses of variance among different groups (genotypes by ARDS status), with adjustment of age, gender, pulmonary injury, pre-admission steroid use, septic shock, and APACHE III score on ICU admission, as well as Bonferroni correction for multiple comparison, were consistent with the observations in subgroup analyses (Table 21).

Among patients with pulmonary injury, controls had significantly higher PI3 plasma levels (n=30; adjusted mean=81.7%; 95% CI: 60.2-110.8 ng/ml) compared to ARDS patients (n=54; adjusted mean=44.6; 95% CI: 35.6-55.9 ng/ml; P=0.001). No difference in plasma PI3 levels between ARDS cases and controls was found in the subgroup with extrapulmonary injury (P=0.480). Moreover, there were no significant differences in PI3 levels between pulmonary vs. extrapulmonary injury among ARDS cases or controls (P=0.780 and P=0.097, respectively). Further stratified analysis to investigate the association between genotypes and plasma PI3 level was not conducted due to the limitation of small sample size in subgroups with variant allele of rs2665481.

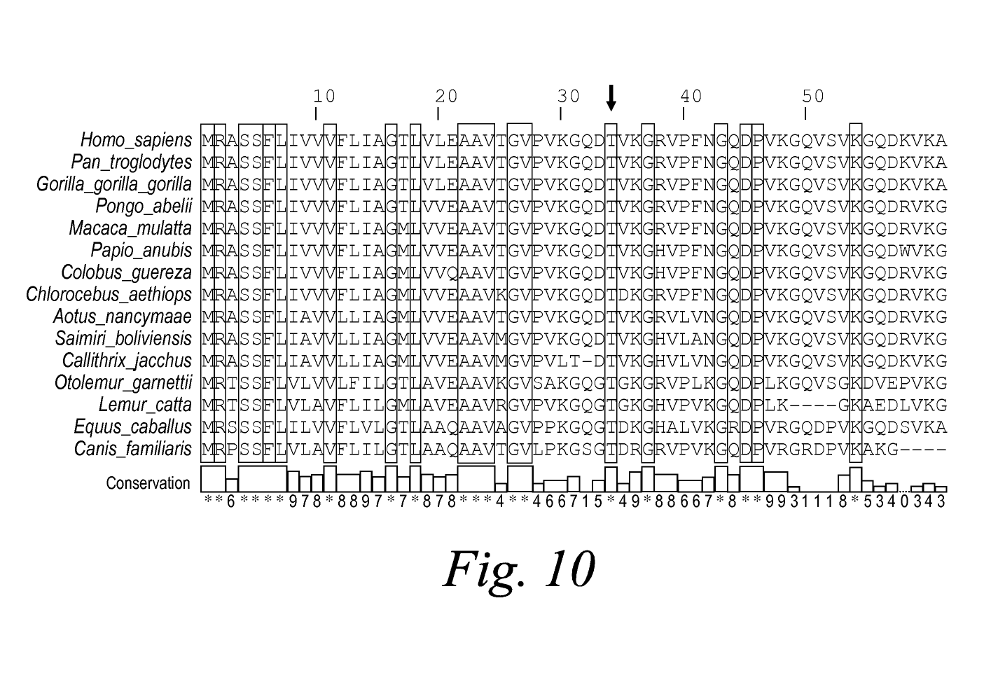

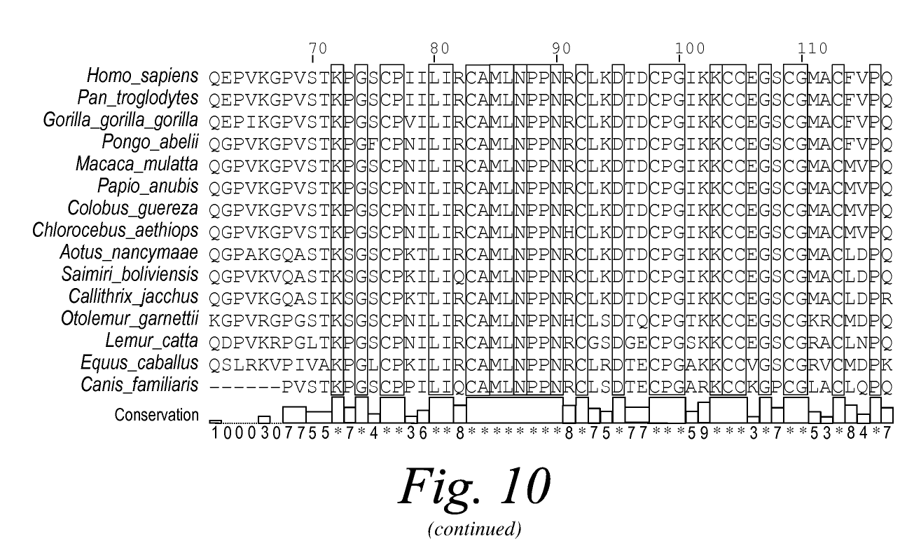

It was found that, at the onset of ARDS, plasma PI3 levels were affected by different genotypes of rs2664581 (T34P) polymorphism. ARDS patients homozygous for A allele had a significantly lower PI3 plasma levels than those with variant C allele. Moreover, among patients homozygous for A allele, ARDS cases had significant lower plasma PI3 levels as compared with the at-risk controls. Our previous studies showed that PI3 plays a protective role against ARDS development. However, the C allele in rs2665481 associated with higher odds of developing ARDS is also associated with higher plasma PI3 in ARDS patients. Without being bound by a single theory, a possible explanation for this apparent contradiction is that the amino acid substitution associated with the C allele results in a defective protein. The SNP rs2664581 is a non-synonymous polymorphism located in exon 2 which results in an amino acid substitution (T→P) within the consensus sequence GQDPVK of the first transglutaminase substrate motif of PI3 (Schalkwijk, J., et al. 1999. Biochem. J., 340 Pt 3., 569-577). The transglutaminase substrate motifs allows the PI3 molecule covalently bind to the ECM proteins and exerting its biological function, making PI3 effective as a tissue-bound inhibitor against HNE (Guyot, N., et al. 2005. Biochemistry, 44, 15610-15618). Although the substitution T→P is located at a non-conservative position of transglutaminase substrate motifs across different protein families, and the second and fifth transglutaminase substrate motifs of PI3 had the same proline at this position, a threonine at this position (amino acid 34) is well conserved in elafins across all species (see FIG. 10). Moreover, the first transglutaminase substrate motif is close to a signal peptide cleavage site so that this motif may be more easily accessed to transglutaminase (Moreau, T., et al. 2008. Biochimie, 90, 284-295; Schalkwijk, J., et al. 1999. Biochem. J., 340 Pt 3., 569-577). Thus, without being bound by any particular theory, this motif may be more important than the rest of the transglutaminase motifs in binding to ECM proteins, and is more sensitive to proline substitution. Proline is known to break a helix of three-dimensional protein structure with significant conformational changes. It is possible that the structure disruption caused by the T34P substitution affects the ability of the PI3 protein to stay bound to ECM protein or else increases the ability of pre-elafin to be cleaved into elafin, which results in a reduction of effective local protection by increasing free PI3 in local tissue and circulation. Further studies should be conducted to explore the functional significance of rs2665481 polymorphism in ARDS.

In the stratified analysis by type of lung injury, it was found that controls had a higher plasma PI3 levels than ARDS in the pulmonary injury group. According to the genotype analysis, the genetic association with ARDS is driven by extrapulmonary injury. However, no differences in plasma PI3 levels between ARDS and controls were found among the extrapulmonary injury group. The small size of ARDS patients with extrapulmonary injury (n=11), could be a possible explanation for the lack of association in this group.

### Discussion of Examples 10-11

The results described above seemed to point the non-synonymous polymorphism rs2664581 (T34P) as a putative causal SNP in ARDS development. The haplotype analysis showed that rs2665481 is independently associated with increased ARDS risk. However, the LD structure around PI3 gene complicates the issue (FIG. 7B). Similar to a previous study in African-American population (Chowdhury, M. A., et al. 2006. BMC Med. Genet., 7, 49), the PI3 gene in non-Hispanic Caucasian population was also highly polymorphic. All polymorphisms were located within LD blocks with high degree of linkage disequilibrium. There were 9 polymorphisms located in the promoter region, with potential differential binding for at least one transcription factor, including, Adf-2a, TBF1, NFATC2, AP1, and GATA1 (Chowdhury, M. A., et al. 2006. BMC Med. Genet., 7, 49; Bingle, L., Tetley, T. D. and Bingle, C.D. 2001. Am. J. Respir. Cell Mol. Biol., 25, 84-91; Pol, A., et al. 2003. J. Invest. Dermatol., 120, 301-307; Zhang, M., et al. 1997. Cancer Res., 57, 4631-4636). Most interestingly, all of them were in complete linkage disequilibrium with rs2664581 (T34P) polymorphism. Without being bound by any theory, it is possible that PI3 plasma levels could be influenced by SNPs in the promoter region, which could modify gene transcription by altering the binding of transcription factors to promoter or by altering the local chromatin architecture. Chowdhury et al identified two SNPs in the PI3 promoter (−1063G→A, not identified in our population, and rs35476703) which showed differential binding of transcription factors in nuclear extracts derived from both amnion and HeLa cells. The SNP rs35476703 site at −689C→G binds to GATA1, suggesting the involvement of this transcription factor in regulation of PI3 in amnion cells. This SNP was not genotyped because it is not a tSNP based on disequilibrium criteria, and genotyping this SNP would not provide additional information because they are essentially in complete LD with SNP rs2664581 (T34P). SNPs in 3′-untranslated region of the PI3 gene, in high LD with rs2664581 could also alter message stability. Therefore, without being bound to any theory it is likely that PI3 polymorphisms had multiple effects at transcriptional, translational, and post-translational levels, any of which may play important roles in the development of ARDS. Furthermore, it is contemplated that additional functional SNPs could be located outside the PI3 locus in LD with tSNP rs2664581 (T34P).

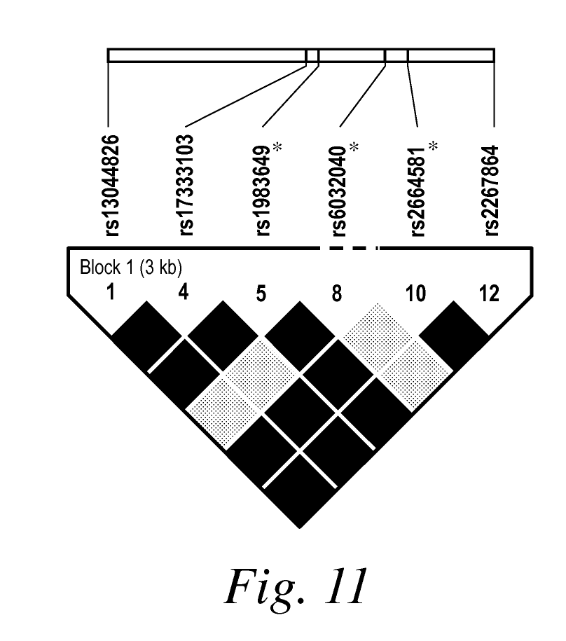

This is the first study to examine the role of genetic variations in PI3 gene in the predisposition to ARDS. This study's results were supported by biologic plausibility that PI3 functioned as a specific inhibitor of HNE (Moreau, T., et al. 2008. Biochimie, 90, 284-295), and was supported by findings described here which pointed PI3 gene as a candidate for ARDS susceptibility (Wang, Z., et al. 2008. Am. J. Respir. Cell. Mol. Biol., 38, 724-732). Several approaches were applied to reduce potential selection biases and confounding factors in the hospital-based study design. Firstly, the cases were strictly defined according to AECC definition for ARDS (Bernard, G. R., et al. 1994. Am. J. Respir. Crit. Care Med., 149, 818-824). Secondly, the selection of controls from at-risk critically ill patients reduced the potential confounding from associations between candidate polymorphisms and predisposing conditions for ARDS. Thirdly, all subjects in this study belonged to a single ethnic group, thus the potential confounding effect due to population stratification is minimized. In addition, this study included 449 ARDS cases and 1031 controls, representing the largest population used in an ARDS association study and provided high statistical power to detect genetic effects on ARDS. The study also had a complete coverage of all common polymorphisms of PI3 gene to ensure the comprehensive evaluation of the association of PI3 polymorphisms with ARDS risk. The tSNPs selection was based on our resequencing data which captured all common variants surrounding the PI3 gene with a r2≧0.8, and was consistent with those predicted by the HapMap data of CEU sample (release 22, on NCBI B36, dbSNP b126. hapmap.org/cgi-perl/gbrowse/hapmap22_B36) (see FIG. 11)

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

TABLE 1
Patient characteristics for subjects with RNA samples for microarray analysis

| | | | | | | | Days |
|---|---|---|---|---|---|---|---|
| | | | | | APACHE | | between |
| Patient # | Group | Age | Gender | Etiology of ARDS | III | Outcome | samples^{a} |
| 1 | ARDS | 57 | Female | Septic Shock | 79 | Alive | 3 |
| 2 | ARDS | 62 | Female | Pneumonia/Septic | 70 | Alive | 21 |
| | | | | Shock | | | |
| 3 | ARDS | 20 | Female | Pneumonia/Septic | 71 | Alive | 14 |
| | | | | Shock | | | |
| 4 | ARDS | 83 | Male | Septic Shock | 119 | Died | 20 |
| 5 | ARDS | 79 | Male | Septic Shock | 99 | Died | 2 |
| 6 | ARDS | 28 | Male | Septic Shock | 117 | Alive | 5 |
| 7 | ARDS | 35 | Male | Septic Shock | 93 | Alive | 16 |
| 8 | ARDS | 29 | Male | Pneumonia/Septic | 55 | Alive | 21 |
| | | | | Shock | | | |
| 9 | ARDS | 37 | Male | Septic Shock | 86 | Alive | 5^{b} |
| 10 | Control | 83 | Male | Septic Shock | 88 | Alive | 6 |
| 11 | Control | 63 | Male | ND | 66 | ND | 3 |

^{a}Days between the collections of two RNA samples.
^{b}Since the acute-stage RNA sample was collected on Day −2, prior to ARDS diagnosis, this patient was excluded from microarray analysis.

TABLE 2
Genes with >1.5 fold-change in expression in peripheral blood between
the acute-stage and recovery-stage of ARDS.

| | | | Gene | Fold |
|---|---|---|---|---|
| Symbol | Probe Set | Gene Name | ID | Change^{a} |
| PI3 | 203691_at | peptidase inhibitor 3, skin-derived | 5266 | −2.98 |
| | 41469_at | (SKALP) | | −2.65 |
| IL8 | 202859_x_at | interleukin 8 | 3576 | −2.93 |
| | 205592_at | | | −1.79 |
| Unknown | 211781_x_at | unknown (gb: BC006164.1) | | −2.22 |
| MME | 203435_s_at | membrane metallo-endopeptidase (neutral | 4311 | −2.12 |
| | | endopeptidase, enkephalinase) | | |
| PTGS2 | 204748_at | prostaglandin-endoperoxide synthase 2 | 5743 | −2.03 |
| | | (prostaglandin G/H synthase and | | |
| | | cyclooxygenase) | | |
| SGK | 201739_at | serum/glucocorticoid regulated kinase | 6446 | −2.03 |
| BNIP3L | 221478_at | BCL2/adenovirus E1B 19 kDa interacting | 665 | −1.84 |
| | | protein 3-like | | |
| POLB | 203616_at | polymerase (DNA directed), beta | 5423 | −1.83 |
| STAT1 | 200887_s_at | signal transducer and activator of | 6772 | −1.78 |
| | | transcription 1, 91 kDa | | |
| FGL2 | 204834_at | fibrinogen-like 2 | 10875 | −1.68 |
| GPR177 | 221958_s_at | G protein-coupled receptor 177 | 79971 | −1.58 |
| PIGF | 205077_s_at | phosphatidylinositol glycan anchor | 5281 | −1.56 |
| | | biosynthesis, class F | | |
| CLEC7A | 221698_s_at | C-type lectin domain family 7, member A | 64581 | −1.56 |
| C14orf159 | 218298_s_at | chromosome 14 open reading frame 159 | 80017 | −1.56 |
| TBCC | 202495_at | tubulin folding cofactor C | 6903 | −1.56 |
| LY75 | 205668_at | lymphocyte antigen 75 | 4065 | −1.55 |
| BTRC | 216091_s_at | beta-transducin repeat containing | 8945 | −1.55 |
| DUSP6 | 208892_s_at | dual specificity phosphatase 6 | 1848 | −1.52 |
| HIP2 | 202346_at | huntingtin interacting protein 2 | 3093 | 1.55 |
| OSBPL1A | 208158_s_at | oxysterol binding protein-like 1A | 114876 | 1.58 |
| STXBP2 | 209367_at | syntaxin binding protein 2 | 6813 | 1.62 |
| IDI1 | 204615_x_at | isopentenyl-diphosphate delta isomerase 1 | 3422 | 1.58 |
| | 208881_x_at | | | 1.65 |
| HSPA1A/ | 200800_s_at | heat shock 70 kDa protein 1A/heat shock | 3303/ | 1.66 |
| HSPA1B | | 70 kDa protein 1B | 3304 | |
| GALNT2 | 217788_s_at | UDP-N-acetyl-alpha-D- | 2590 | 1.83 |
| | | galactosamine:polypeptide N- | | |
| | | acetylgalactosaminyltransferase 2 | | |
| | | (GalNAc-T2) | | |
| PDGFC | 218718_at | platelet derived growth factor C | 56034 | 2.08 |
| HPGD | 203914_x_at | hydroxyprostaglandin dehydrogenase 15- | 3248 | 2.00 |
| | 203913_s_at | (NAD) | | 2.29 |
| GADD45A | 203725_at | growth arrest and DNA-damage-inducible, | 1647 | 2.38 |
| | | alpha | | |

^{a}A negative number of fold change means the gene was down-regulated during the acute-stage of ARDS, and a positive number of fold change means the gene was up-regulated during the acute-stage.

TABLE 3
Gene ontology categories with enrichment of 126 differentially-expressed
genes^{a}

| | | | # genes | # genes |
|---|---|---|---|---|
| GO ID | GO Name | p-value | in list | in array |
| 50896 | response to stimulus | 1.52E−06 | 36 | 1672 |
| 6950 | response to stress | 1.14E−06 | 25 | 901 |
| 9607 | response to biotic stimulus | 6.69E−06 | 22 | 802 |
| 6952 | defense response | 3.77E−05 | 20 | 766 |
| 51707 | response to other organism | 5.11E−05 | 15 | 478 |
| 6955 | immune response | 2.93E−05 | 19 | 690 |
| 9613 | response to pest, pathogen or | 4.02E−05 | 15 | 468 |
| | parasite | | | |
| 16265 | Death | 3.20E−07 | 19 | 506 |
| 8219 | cell death | 3.01E−07 | 19 | 504 |
| 12501 | programmed cell death | 6.49E−07 | 18 | 478 |
| 6915 | apoptosis | 6.10E−07 | 18 | 476 |
| 43067 | regulation of programmed | 5.83E−05 | 12 | 321 |
| | cell death | | | |
| 42981 | regulation of apoptosis | 5.49E−05 | 12 | 319 |

^{a}All GO biological processes were significantly enriched in the lists of 126 genes identified by paired t-test (p < 0.05) after multiple comparison adjustment (Bonferroni correction).

TABLE 4
Characterization of Study Population for ELISA Assay

| | ARDS | Control | p |
|---|---|---|---|
| | (n = 40) | (n = 23) | Value |
| General | | | |
| Age, year (mean ± SD) | 60.5 ± 17.3 | 57.7 ± 9.2 | 0.407 |
| Gender, (male/female) | 12/11 | 24/16 | 0.547 |
| APACHE III score, (mean ± SD) | 79.7 ± 24.3 | 69.7 ± 15.4 | 0.052 |
| Thrombocytopenia | 9 (22.5) | 4 (17.4) | 0.753 |
| (platelets<80,000 mm⁻³), N (%) | | | |
| Creatinine >2.0 mg/dL, N (%) | 16 (40.0) | 6 (26.1) | 0.265 |
| Total bilirubin >2.0 mg/dL, N (%) | 8 (20.0) | 3 (13.0) | 0.732 |
| Transfusion of PRBC, N (%) | 29 (72.5) | 9 (39.1) | 0.009 |
| Number of PRBC transfused | 4.8 ± 6.3 | 3.0 ± 5.7 | 0.255 |
| Liver disease, N (%) | 4 (10.0) | 1 (4.4) | 0.644 |
| End-stage renal disease, N (%) | 3 (7.5) | 1 (4.4 | 1.000 |
| Diabetes, N (%) | 10 (25.0) | 7 (30.4) | 0.640 |
| Recent steroid use, N (%) | 5 (12.5) | 4 (17.4) | 0.713 |
| CLINICAL RISK FACTORS, N (%) | | | |
| Direct lung injury | 20 (50.0) | 11 (47.8) | 0.868 |
| Septic shock | 22 (55.0) | 8 (34.8) | 0.122 |
| Sepsis | 13 (32.5) | 10 (43.5) | 0.384 |
| Trauma | 2 (5.0) | 1 (4.4) | 1.000 |
| Multiple transfusion | 7 (17.5) | 2 (8.7) | 0.467 |

TABLE 5
Complete list of 126 genes with changed expression in peripheral blood
between the date of ARDS diagnosis and the date of ICU discharge
(paired t-test, p < 0.05)

| | | | | | Med- |
|---|---|---|---|---|---|
| | | | | Fold | Gene |
| Symbol | Probe Set | Gene Name | Gene ID | Change^{a} | Category^{b} |
| PI3 | 203691_at | peptidase inhibitor 3, skin-derived (SKALP) | 5266 | −2.98 | 1 |
| | 41469_at | | | −2.65 | |
| IL8 | 202859_x_at | interleukin 8 | 3576 | −2.93 | 1 |
| | 205592_at | | | −1.79 | |
| unknown | 211781_x_at | unknown (gb: BC006164.1) | | −2.22 | |
| MME | 203435_s_at | membrane metallo-endopeptidase (neutral | 4311 | −2.12 | 3 |
| | | endopeptidase, enkephalinase) | | | |
| PTGS2 | 204748_at | prostaglandin-endoperoxide synthase 2 | 5743 | −2.03 | 1 |
| | | (prostaglandin G/H synthase and | | | |
| | | cyclooxygenase) | | | |
| SGK | 201739_at | serum/glucocorticoid regulated kinase | 6446 | −2.03 | 3 |
| BNIP3L | 221478_at | BCL2/adenovirus E1B 19 kDa interacting | 665 | −1.84 | 3 |
| | | protein 3-like | | | |
| POLB | 203616_at | polymerase (DNA directed), beta | 5423 | −1.83 | 3 |
| STAT1 | 200887_s_at | signal transducer and activator of transcription | 6772 | −1.78 | 3 |
| | | 1, 91 kDa | | | |
| FGL2 | 204834_at | fibrinogen-like 2 | 10875 | −1.68 | 3 |
| GPR177 | 221958_s_at | G protein-coupled receptor 177 | 79971 | −1.58 | 3 |
| PIGF | 205077_s_at | phosphatidylinositol glycan anchor | 5281 | −1.56 | 3 |
| | | biosynthesis, class F | | | |
| CLEC7A | 221698_s_at | C-type lectin domain family 7, member A | 64581 | −1.56 | 3 |
| C14orf159 | 218298_s_at | chromosome 14 open reading frame 159 | 80017 | −1.56 | 4 |
| TBCC | 202495_at | tubulin folding cofactor C | 6903 | −1.56 | 4 |
| LY75 | 205668_at | lymphocyte antigen 75 | 4065 | −1.55 | 3 |
| BTRC | 216091_s_at | beta-transducin repeat containing | 8945 | −1.55 | 3 |
| DUSP6 | 208892_s_at | dual specificity phosphatase 6 | 1848 | −1.52 | 3 |
| ZYG11BL | 202448_s_at | zyg-11 homolog B (C. elegans)-like | 10444 | −1.50 | 4 |
| ZNF589 | 210062_s_at | zinc finger protein 589 | 51385 | −1.48 | 3 |
| ZNF133 | 216960_s_at | zinc finger protein 133 | 7692 | −1.46 | 3 |
| ZCCHC10 | 221193_s_at | zinc finger, CCHC domain containing 10 | 54819 | −1.42 | 4 |
| XK | 206698_at | X-linked Kx blood group (McLeod syndrome) | 7504 | −1.42 | 4 |
| WARS | 200629_at | tryptophanyl-tRNA synthetase | 7453 | −1.42 | 3 |
| VPS13C | 218396_at | vacuolar protein sorting 13 homolog C (S. cerevisiae) | 54832 | −1.41 | 4 |
| VDAC1 | 217140_s_at | voltage-dependent anion channel 1 | 7416 | −1.41 | 3 |
| UBE2L6 | 201649_at | ubiquitin-conjugating enzyme E2L 6 | 9246 | −1.41 | 4 |
| TUBB2A | 204141_at | tubulin, beta 2A | 7280 | −1.40 | 4 |
| TRGC2/ | 215806_x_at | T cell receptor gamma constant 2/T cell | 445347/ | −1.40 | 3 |
| TRGV9/ | 209813_x_at | receptor gamma variable 9/TCR gamma | 6967/ | −1.39 | |
| TARP | | alternate reading frame protein | 6983 | | |
| TRBV21-1/ | 211796_s_at | T cell receptor beta variable 21-1/T cell | 28566/ | −1.39 | 4 |
| TRBV19/ | | receptor beta variable 19/T cell receptor beta | 28568/ | | |
| TRBV7-2/ | | variable 7-2/T cell receptor beta variable 5-4/ | 28596/ | | |
| TRBV5-4/ | | T cell receptor beta variable 3-1/T cell | 28611/ | | |
| TRBV3-1/ | | receptor beta constant 1 | 28619/ | | |
| TRBC1 | | | 28639 | | |
| TRBV19/ | 210915_x_at | T cell receptor beta variable 19/T cell receptor | 28568/ | −1.39 | 4 |
| TRBC1 | | beta constant 1 | 28639 | | |
| TRAK2 | 202124_s_at | trafficking protein, kinesin binding 2 | 66008 | −1.38 | 3 |
| TRAK1 | 202079_s_at | trafficking protein, kinesin binding 1 | 22906 | −1.38 | 3 |
| TRAC | 209670_at | T cell receptor alpha constant | 28755 | −1.38 | 3 |
| TRA@ | 211902_x_at | T cell receptor alpha locus | 6955 | −1.37 | 3 |
| TRA@/ | 210972_x_at | T cell receptor alpha locus/T cell receptor | 28517/ | −1.37 | 4 |
| TRDV2/ | | delta variable 2/T cell receptor alpha variable | 28663/ | | |
| TRAV20/ | | 20/T cell receptor alpha joining 17/T cell | 28738/ | | |
| TRAJ17/ | | receptor alpha constant | 28755/ | | |
| TRAC | | | 6955 | | |
| TNS1 | 221748_s_at | tensin 1 | 7145 | −1.37 | 3 |
| TNFSF10 | 202688_at | tumor necrosis factor (ligand) superfamily, | 8743 | −1.36 | 3 |
| | | member 10 | | | |
| TNFAIP2 | 202510_s_at | tumor necrosis factor, alpha-induced protein 2 | 7127 | −1.36 | 3 |
| TMEM158 | 213338_at | transmembrane protein 158 | 25907 | −1.36 | 4 |
| TLE4 | 216997_x_at | transducin-like enhancer of split 4 (E(sp1) | 7091 | −1.34 | 3 |
| | | homolog, Drosophila) | | | |
| THBS1 | 215775_at | Thrombospondin 1 | 7057 | −1.34 | 1 |
| TAF9 | 202168_at | TAF9 RNA polymerase II, TATA box binding | 6880 | −1.33 | 3 |
| | | protein (TBP)-associated factor, 32 kDa | | | |
| STK19 | 36019_at | serine/threonine kinase 19 | 8859 | −1.31 | 2 |
| SRPRB | 218140_x_at | signal recognition particle receptor, B subunit | 58477 | −1.31 | 4 |
| SOD2 | 215223_s_at | superoxide dismutase 2, mitochondrial | 6648 | −1.31 | 1 |
| SMCHD1 | 212569_at | structural maintenance of chromosomes flexible | 23347 | −1.30 | 4 |
| | 212577_at | hinge domain containing 1 | | −1.30 | |
| | 212579_at | | | −1.30 | |
| SLC16A5 | 213590_at | solute carrier family 16, member 5 | 9121 | −1.30 | 4 |
| | | (monocarboxylic acid transporter 6) | | | |
| SETDB1 | 214197_s_at | SET domain, bifurcated 1 | 9869 | −1.30 | 3 |
| SELENBP1 | 214433_s_at | selenium binding protein 1 | 8991 | −1.29 | 3 |
| SCO2 | 205241_at | SCO cytochrome oxidase deficient homolog 2 | 9997 | −1.29 | 3 |
| | | (yeast) | | | |
| RUNX3 | 204197_s_at | runt-related transcription factor 3 | 864 | −1.29 | 3 |
| | 204198_s_at | | | −1.28 | |
| RP3- | 222279_at | hypothetical protein FLJ35429 | 285830 | −1.28 | 4 |
| 377H14.5 | | | | | |
| RARRES3 | 204070_at | retinoic acid receptor responder (tazarotene | 5920 | −1.27 | 3 |
| | | induced) 3 | | | |
| RAD9A | 204828_at | RAD9 homolog A (S. pombe) | 5883 | −1.27 | 3 |
| RAD17 | 207405_s_at | RAD17 homolog (S. pombe) | 5884 | −1.26 | 3 |
| PSME2 | 201762_s_at | proteasome (prosome, macropain) activator | 5721 | −1.26 | 3 |
| | | subunit 2 (PA28 beta) | | | |
| PSME1 | 200814_at | proteasome (prosome, macropain) activator | 5720 | −1.26 | 3 |
| | | subunit 1 (PA28 alpha) | | | |
| PPP2R1B | 202884_s_at | protein phosphatase 2 (formerly 2A), regulatory | 5519 | −1.26 | 3 |
| | | subunit A (PR 65), beta isoform | | | |
| POLS | 202466_at | polymerase (DNA directed) sigma | 11044 | −1.26 | 3 |
| PLA2G7 | 206214_at | phospholipase A2, group VII (platelet- | 7941 | −1.26 | 2 |
| | | activating factor acetylhydrolase, plasma) | | | |
| PDE4B | 203708_at | phosphodiesterase 4B, cAMP-specific | 5142 | −1.25 | 3 |
| | 222326_at | (phosphodiesterase E4 dunce homolog, | | −1.25 | |
| | | Drosophila) | | | |
| PDCD4 | 212594_at | programmed cell death 4 (neoplastic | 27250 | −1.25 | 3 |
| | | transformation inhibitor) | | | |
| PCAF | 203845_at | p300/CBP-associated factor | 8850 | −1.25 | 3 |
| PBLD | 219543_at | phenazine biosynthesis-like protein domain | 64081 | −1.25 | 3 |
| | | containing | | | |
| PARVB | 37965_at | parvin, beta | 29780 | −1.24 | 4 |
| PAIP1 | 213754_s_at | poly(A) binding protein interacting protein 1 | 10605 | −1.24 | 3 |
| P2RY5 | 218589_at | purinergic receptor P2Y, G-protein coupled, 5 | 10161 | −1.24 | 3 |
| OPTN | 202074_s_at | optineurin | 10133 | −1.24 | 3 |
| ODC1 | 200790_at | ornithine decarboxylase 1 | 4953 | −1.24 | 3 |
| OCLM | 208274_at | oculomedin | 10896 | −1.24 | 4 |
| NPC2 | 200701_at | Niemann-Pick disease, type C2 | 10577 | −1.24 | 3 |
| NOV | 214321_at | nephroblastoma overexpressed gene | 4856 | −1.23 | 3 |
| NOD2 | 220066_at | nucleotide-binding oligomerization domain | 64127 | −1.23 | 3 |
| | | containing 2 | | | |
| NFATC2IP | 217527_s_at | nuclear factor of activated T-cells, cytoplasmic, | 84901 | −1.23 | 4 |
| | | calcineurin-dependent 2 interacting protein | | | |
| NBPF1/ | 215434_x_at | neuroblastoma breakpoint family, member 1/ | 440673/ | −1.23 | 3 |
| NBPF10 | | neuroblastoma breakpoint family, member 10 | 55672 | | |
| NAPA | 206491_s_at | N-ethylmaleimide-sensitive factor attachment | 8775 | −1.23 | 3 |
| | | protein, alpha | | | |
| NAP1L4 | 201414_s_at | nucleosome assembly protein 1-like 4 | 4676 | −1.22 | 3 |
| MX1 | 202086_at | myxovirus (influenza virus) resistance 1, | 4599 | −1.22 | 3 |
| | | interferon-inducible protein p78 (mouse) | | | |
| MULK | 222132_s_at | multiple substrate lipid kinase | 55750 | −1.22 | 3 |
| MRPS18A | 218385_at | mitochondrial ribosomal protein S18A | 55168 | −1.22 | 4 |
| MMD | 203414_at | monocyte to macrophage differentiation- | 23531 | −1.22 | 3 |
| | | associated | | | |
| METTL4 | 219698_s_at | methyltransferase like 4 | 64863 | −1.22 | 4 |
| MBOAT2 | 213288_at | membrane bound O-acyltransferase domain | 129642 | −1.21 | 4 |
| | | containing 2 | | | |
| MBD4 | 214048_at | methyl-CpG binding domain protein 4 | 8930 | −1.21 | 3 |
| MARCH8 | 221824_s_at | membrane-associated ring finger (C3HC4) 8 | 220972 | −1.21 | 3 |
| MAN2A2 | 219999_at | mannosidase, alpha, class 2A, member 2 | 4122 | −1.21 | 4 |
| LOC646912 | 217092_x_at | similar to 60S ribosomal protein L7 | 646912 | −1.21 | 4 |
| LOC54103 | 222150_s_at | hypothetical protein LOC54103 | 54103 | −1.21 | 4 |
| CLEC2B/ | 209732_at | C-type lectin domain family 2, member B/ | 94158/ | 1.21 | 4 |
| CDRT15P | | CMT1A duplicated region transcript 15 | 9976 | | |
| | | pseudogene | | | |
| CLCN5 | 206704_at | chloride channel 5 (nephrolithiasis 2, X-linked, | 1184 | 1.22 | 3 |
| | | Dent disease) | | | |
| CLC | 206207_at | Charcot-Leyden crystal protein | 1178 | 1.22 | 3 |
| CIR | 209571_at | CBF1 interacting corepressor | 9541 | 1.23 | 3 |
| CHRNE | 215916_at | cholinergic receptor, nicotinic, epsilon | 1145 | 1.23 | 4 |
| CECR1 | 219505_at | cat eye syndrome chromosome region, | 51816 | 1.23 | 3 |
| | | candidate 1 | | | |
| CDC2L1/ | 211289_x_at | cell division cycle 2-like 1 (PITSLRE proteins)/ | 984/ | 1.23 | 3 |
| CDC2L2 | | cell division cycle 2-like 2 (PITSLRE | 985 | | |
| | | proteins) | | | |
| CD74 | 209619_at | CD74 molecule, major histocompatibility | 972 | 1.24 | 3 |
| | | complex, class II invariant chain | | | |
| CD52 | 34210_at | CD52 molecule | 1043 | 1.24 | 3 |
| CD3E | 205456_at | CD3e molecule, epsilon (CD3-TCR complex) | 916 | 1.26 | 3 |
| CD36 | 209555_s_at | CD36 molecule (thrombospondin receptor) | 948 | 1.26 | 3 |
| CD2 | 205831_at | CD2 molecule | 914 | 1.26 | 1 |
| CD14 | 201743_at | CD14 molecule | 929 | 1.27 | 1 |
| CCL5 | 204655_at | chemokine (C—C motif) ligand 5 | 6352 | 1.30 | 3 |
| CASP9 | 203984_s_at | caspase 9, apoptosis-related cysteine peptidase | 842 | 1.30 | 3 |
| C1orf50 | 62212_at | chromosome 1 open reading frame 50 | 79078 | 1.31 | 4 |
| BTN3A3 | 38241_at | butyrophilin, subfamily 3, member A3 | 10384 | 1.32 | 4 |
| BLVRB | 202201_at | biliverdin reductase B (flavin reductase | 645 | 1.33 | 3 |
| | | (NADPH)) | | | |
| BCL2L1 | 215037_s_at | BCL2-like 1 | 598 | 1.34 | 3 |
| BAG1 | 202387_at | BCL2-associated athanogene | 573 | 1.36 | 3 |
| ATP2B1 | 209281_s_at | ATPase, Ca++ transporting, plasma membrane 1 | 490 | 1.37 | 3 |
| ATG4B | 204903_x_at | ATG4 autophagy related 4 homolog B (S. cerevisiae) | 23192 | 1.38 | 3 |
| ASCC2 | 215684_s_at | activating signal cointegrator 1 complex | 84164 | 1.39 | 4 |
| | | subunit 2 | | | |
| ARL4C | 202207_at | ADP-ribosylation factor-like 4C | 10123 | 1.39 | 3 |
| APOBEC3G | 204205_at | apolipoprotein B mRNA editing enzyme, | 60489 | 1.40 | 3 |
| | | catalytic polypeptide-like 3G | | | |
| APBB1IP | 219994_at | amyloid beta (A4) precursor protein-binding, | 54518 | 1.40 | 3 |
| | | family B, member 1 interacting protein | | | |
| AMPD2 | 212360_at | adenosine monophosphate deaminase 2 | 271 | 1.46 | 3 |
| | | (isoform L) | | | |
| ABLIM1 | 210461_s_at | actin binding LIM protein 1 | 3983 | 1.47 | 3 |
| ABCA1 | 203505_at | ATP-binding cassette, sub-family A (ABC1), | 19 | 1.48 | 3 |
| | | member 1 | | | |
| HIP2 | 202346_at | huntingtin interacting protein 2 | 3093 | 1.55 | 3 |
| OSBPL1A | 208158_s_at | oxysterol binding protein-like 1A | 114876 | 1.58 | 4 |
| STXBP2 | 209367_at | syntaxin binding protein 2 | 6813 | 1.62 | 4 |
| IDI1 | 204615_x_at | isopentenyl-diphosphate delta isomerase 1 | 3422 | 1.58 | 3 |
| | 208881_x_at | | | 1.65 | |
| HSPA1A/ | 200800_s_at | heat shock 70 kDa protein 1A/heat shock | 3303/ | 1.66 | 1 |
| HSPA1B | | 70 kDa protein 1B | 3304 | | |
| GALNT2 | 217788_s_at | UDP-N-acetyl-alpha-D- | 2590 | 1.83 | 3 |
| | | galactosamine:polypeptide N- | | | |
| | | acetylgalactosaminyltransferase 2 (GalNAc-T2) | | | |
| PDGFC | 218718_at | platelet derived growth factor C | 56034 | 2.08 | 3 |
| HPGD | 203914_x_at | hydroxyprostaglandin dehydrogenase 15- | 3248 | 2.00 | 3 |
| | 203913_s_at | (NAD) | | 2.29 | |
| GADD45A | 203725_at | growth arrest and DNA-damage-inducible, | 1647 | 2.38 | 3 |
| | | alpha | | | |

^{a}A negative number of fold change means the gene was down-regulated during the acute-stage of ARDS, and a positive number of fold change means the gene was up-regulated during the acute-stage.
^{b}The gene list was sorted in MedGene database by the linkage of genes and ARDS. 1: first-degree association, genes that have been directly linked to ARDS by gene term search; 2: first-degree associations by family term, genes that have been directly linked to this disease by family term search; 3: second degree associations, genes that have never been co-cited with ARDS, but have been linked to at least one first-degree gene; and 4: Genes new to ARDS, genes that have not been previously associated with this disease.

TABLE 6
Literature mining of ARDS-related genes in Medline Database against 28
altered genes identified by microarray analysis

| | Top | Genes Identified from Microarray Analysis^{d} | | |
|---|---|---|---|---|
| | Ranked | | Down-regulated at acute | Up-regulated at acute |
| | Genes^{c} | Total | stage | stage |
| Disease^{a} | | | | |
| ARDS | 100 | 7 | IL8, PI3 | CD14 |
| MeSH Vocabulary^{b} | | | | |
| Neutrophil | 538 | 22 | IL8, LY75, MBD4, PCAF, | ABCA1, CCL5, CD14, |
| | | | PDE4B, PI3, SELENBP1, | CD36, CD74 |
| | | | STAT1, THBS1, VDAC1, | |
| | | | WARS | |
| Leukotriene | 419 | 17 | IL8, PI3, PTGS2 | ABCA1, BCL2L1, |
| | | | | CCL5, CD14, HPGD |
| Prostaglandin | 754 | 22 | FGL2, IL8, MMD, OPTN, | ABCA1, CD2, CD36, |
| | | | PDCD4, PDE4B, PIGF, | GADD45A, HPGD, |
| | | | POLB, PTGS2, STAT1, | PDGFC |
| | | | STK19, TNFSF10 | |

^{a}Text mining through MedGene project at Harvard Institute of Proteomics (hip.harvard.edu, by Mar. 14, 2007) for genes associated with ARDS (MeSH term: Respiratory Distress Syndrome, Adult) in the Medline Database.
^{b}Text mining through BioGene project at Harvard Institute of Proteomics (hip.harvard.edu, by Mar. 14, 2007) for genes associated with ARDS-related MeSH vocabularies in the Medline Database.
^{c}The statistical method used to rank the gene list is product of frequency. The project allows downloading maximum number of 100 top-ranked genes for each disease or MeSH vocabulary, without specific request for longer gene list. The numbers of top-ranked genes of MeSH mining are the combined lists of non-redundant genes of all sub-vocabularies available at BioGene (by Mar. 14, 2007).
^{d}Genes with altered expression in microarray analysis were found in ARDS-related text mining.

TABLE 7
Characteristics of the study population

| | ARDS | At-risk controls | |
|---|---|---|---|
| | (n = 148) | (n = 64) | P |
| Age-yr, mean ± SD | 60 ± 19 | 61 ± 17 | 0.730 |
| Gender, male/female | 90/58 | 38/26 | 0.844 |
| Caucasian, n (%) | 140 (94.4) | 61 (95.3) | 0.829 |
| APACHE III score, mean ± SD^{a} | 81 ± 22 | 72 ± 23 | 0.006 |
| Risk factors, n (%) | | | |
| Sepsis | 134 (91.5) | 52 (81.3) | 0.058 |
| Septic shock | 89 (60.1) | 28 (43.8) | 0.028 |
| Pneumonia | 118 (79.7) | 25 (39.1) | <0.001 |
| Aspiration | 14 (9.5) | 6 (9.4) | 0.985 |
| Direct lung injury^{b} | 125 (84.5) | 31 (48.4) | <0.001 |
| Multiple transfusion | 10 (6.8) | 7 (10.9) | 0.304 |
| Trauma | 7 (4.7) | 6 (9.4) | 0.196 |
| Comorbidities, n (%) | | | |
| Diabetes | 28 (18.9) | 15 (23.4) | 0.452 |
| Liver failure/cirrhosis | 10 (6.8) | 3 (4.7) | 0.564 |
| Corticosteroid treatment before | 26 (17.6) | 4 (6.3) | 0.030 |
| ICU admission, n (%)^{c} | | | |

ARDS, acute respiratory distress syndrome;
APACHE, Acute Physiology and Chronic Health Evaluation;
^{a}APACHE III physiology score was calculated with all components on the day of ICU admission;
^{b}Pneumonia, aspiration, pulmonary contusions, or sepsis from lower pulmonary source were categorized as direct lung injury. Sepsis from an extrapulmonary source, trauma without pulmonary contusions, and multiple transfusions were categorized as indirect lung injury. Patients with both direct and indirect lung injuries were considered to have direct lung injury;
^{c}Patient received ≧300 mg of prednisone or its equivalent within 21 days or ≧15 mg prednisone a day or its equivalent prior to ICU admission.

TABLE 8
Plasma profile comparison between ARDS and controls

| | | | | Healthy |
|---|---|---|---|---|
| | ARDS | Control | | Reference |
| | Mean (95% CI) | Mean (95% CI) | P^{c} | Mean (95% CI)^{d} |
| All samples^{a} | N = 67 | N = 63 | | |
| PI3 | 44.3 (35.0-56.0) | 69.3 (54.4-88.2) | 0.005 | 19.6 (13.9-27.5) |
| SLPI | 115.3 (101.2-131.5) | 132.5 (115.8-151.7) | 0.752 | 69.6 (62.3-77.9) |
| HNE | 643.6 (525.3-788.7) | 453.3 (367.6-558.9) | 0.017 | 77.1 (63.9-93.1) |
| HNE/PI3 ratio | 14.5 (10.9-19.4) | 6.5 (4.9-8.8) | <0.0001 | 3.9 (2.7-5.9) |
| Direct lung injury^{b} | N = 54 | N = 30 | | |
| PI3 | 44.6 (35.6-55.9) | 81.7 (60.2-110.8) | 0.001 | |
| SLPI | 126.2 (112.0-142.3) | 140.7 (119.6-165.4) | 0.221 | |
| HNE | 546.2 (447.5-666.7) | 396.3 (302.6-519.1) | 0.107 | |
| HNE/PI3 ratio | 12.2 (9.3-16.1) | 4.9 (3.3-7.0) | <0.0001 | |
| Indirect lung injury^{b} | N = 13 | N = 33 | | |
| PI3 | 66.7 (36.2-122.9) | 77.9 (44.9-135.3) | 0.480 | |
| SLPI | 108.6 (74.0-159.3) | 136.9 (96.9-193.5) | 0.655 | |
| HNE | 639.9 (376.7-1087.1) | 491.0 (304.5-791.6) | 0.655 | |
| HNE/PI3 ratio | 13.7 (4.5-20.3) | 6.3 (3.2-12.4) | 0.254 | |

ARDS, acute respiratory distress syndrome;
Control, at-risk ICU control;
PI3, neutrophil elastase inhibitor (elafin);
SLPI, secretory leukocyte proteinase inhibitor;
HNE, neutrophil elastase;
the plasma levels of PI3, SLPI, and HNE were shown in unit of ng/ml;
95% CI, 95% confidence interval.
^{a}To account for repeated measurements, mean plasma levels were calculated using mixed models after adjusting for age, gender, type of lung injury, pre-admission steroid use, septic shock, and APACHE III score on ICU admission. Type of lung injury: direct lung injury - pneumonia, aspiration, pulmonary contusions, or sepsis from lower pulmonary source; indirect lung injury - sepsis from an extrapulmonary source, trauma without pulmonary contusions, and multiple transfusions. Patients with both direct and indirect lung injuries were considered to have direct lung injury.
^{b}To account for repeated measurements, mean plasma levels were calculated using mixed models after adjusting for age, gender, pre-admission steroid use, septic shock and APACHE III score on ICU admission.
^{c}P values were calculated between the ARDS and the controls.
^{d}Mean plasma levels of anonymous healthy individuals (reference groups) were calculated directly without any adjustment.

TABLE 9
ANOVA analyses of plasma profiles among the pre-ARDS,
ARDS and the Control^{a}

| | | | | Mean (95% CI) | |
|---|---|---|---|---|---|
| | Control | Pre-ARDS | ARDS | ng/ml^{c} | P^{b} |
| PI3 | | | | | 0.009 |
| Control | NA | ++ | − | 80.3 (66.7-96.6) | |
| Pre-ARDS | | NA | ++ | 87.5 (61.0-125.5) | |
| ARDS | | | NA | 52.9 (42.7-65.6) | |
| SLPI | | | | | 0.187 |
| Control | NA | − | − | 139.4 (126.1-154.0) | |
| Pre-ARDS | | NA | − | 125.2 (103.6-151.4) | |
| ARDS | | | NA | 120.7 (107.6-135.5) | |
| HNE | | | | | 0.012 |
| Control | NA | + | ++ | 447.5 (380.6-526.2) | |
| Pre-ARDS | | NA | − | 643.4 (472.8-875.7) | |
| ARDS | | | NA | 633.0 (524.8-763.6) | |
| HNE/PI3 | | | | | 0.0001 |
| Control | NA | − | ++ | 5.6 (4.4-7.0) | |
| Pre-ARDS | | NA | ++ | 7.5 (4.8-11.7) | |
| ARDS | | | NA | 12.0 (9.2-15.6) | |

ARDS, acute respiratory distress syndrome;
PI3, neutrophil elastase inhibitor (elafin);
SLPI, secretory leukocyte proteinase inhibitor;
HNE, neutrophil elastase;
95% CI, 95% confidence interval.
^{a}ANOVA analysis for PI3 and HNE/PI3 using generalized linear model with adjustment of covariates, including age, gender, type of lung injury, preadmission steroid use, septic shock, PI3 SNP A959C (rs2664581) and APACHE III score on ICU admission. ANOVA analysis for SLPI and HNE using generalized linear model with adjustment of covariates, including age, gender, type of lung injury, pre-admission steroid use, septic shock, and APACHE III score on ICU admission. Pairwise comparison: ++, P < 0.05 after Bonferroni correction for multiple comparisons; +, P < 0.05 without Bonferroni correction; −, P ≧ 0.05; NA, not applicable.
^{b}P values of generalized linear model for all groups.
^{c}The unit of HNE/PI3 is fold changes.

TABLE 10
Changes of plasma profiles between paired samples

| | | | Day 3 after ICU | | |
|---|---|---|---|---|---|
| | ICU Admission^{a} | | Admission^{b} | | |
| | Mean (95% CI) | | Mean (95% CI) | | P^{c} |
| Control (all | | | | | |
| samples, n = 63) | | | | | |
| PI3 | 73.1 | (58.9-90.7) | 76.9 | (63.4-93.3) | 0.335 |
| SLPI | 133.6 | (118.8-150.1) | 119.7 | (106.8-134.2) | 0.019 |
| HNE | 421.8 | (351.5-506.1) | 329.8 | (276-394.1) | 0.003 |
| HNE/PI3 ratio | 5.8 | (4.4-7.6) | 4.3 | (3.3-5.5) | 0.001 |
| Control | | | | | |
| (direct lung | | | | | |
| injury, n = 30) | | | | | |
| PI3 | 88.2 | (62.3-124.7) | 81.6 | (58.4-113.9) | 0.302 |
| SLPI | 147 | (125.4-172.2) | 133.9 | (115.4-155.4) | 0.164 |
| HNE | 351.8 | (268.6-460.7) | 262.7 | (202.5-340.9) | 0.023 |
| HNE/PI3 ratio | 4.0 | (2.6-6.2) | 3.2 | (2.1-4.9) | 0.115 |
| Control | | | | | |
| (indirect lung | | | | | |
| injury, n = 33) | | | | | |
| PI3 | 61.6 | (47-80.7) | 73 | (58.1-91.6) | 0.023 |
| SLPI | 122.4 | (103-145.5) | 108.1 | (91.1-128.3) | 0.063 |
| HNE | 497.4 | (388.8-636.5) | 405.6 | (321.1-512.4) | 0.069 |
| HNE/PI3 ratio | 8.1 | (5.8-11.3) | 5.6 | (4.1-7.5) | 0.004 |
| ARDS (all | | | | | |
| samples, n = 24) | | | | | |
| PI3 | 48.9 | (31.4-76) | 57.5 | (38.9-85.1) | 0.132 |
| SLPI | 122.1 | (101.3-147.1) | 130.5 | (104-163.9) | 0.226 |
| HNE | 561 | (414.4-759.4) | 522.5 | (421.3-648) | 0.552 |
| HNE/PI3 ratio | 11.5 | (7.7-17) | 9.1 | (6.1-13.6) | 0.083 |

PI3, neutrophil elastase inhibitor (elafin);
SLPI, secretory leukocyte proteinase inhibitor;
HNE, neutrophil elastase;
the plasma levels of PI3, SLPI, and HNE were shown in unit of ng/ml;
95% CI, 95% confidence interval.
^{a}For at-risk control patients, the first plasma samples were collected within 48-hour of ICU admission; For ARDS patients, the first plasma samples were collected within 48-hour of ARDS diagnosis.
^{b}The second samples were collected three days after the first collection.
^{c}Paired t-test

TABLE 11
Characteristics of the study population

| | Subjects did | Subjects | |
|---|---|---|---|
| | not develop | developed | |
| Characteristic | ARDS (n = 1031) | ARDS (n = 449) | P |
| Age-yr mean ± SD | 62.78 ± 17 | 59.49 ± 18 | <0.001 |
| Male | 627 (60.8%) | 269 (59.9%) | 0.744 |
| APACHE III score, | 66.46 ± 23 | 77.40 ± 24 | <0.001 |
| mean ± SD | | | |
| Risk factors, n (%) | | | |
| Sepsis | 383 (37.1%) | 117 (26.1%) | <0.001 |
| Septic shock | 448 (43.4%) | 267 (59.5%) | <0.001 |
| Pneumonia | 443 (43.0%) | 302 (67.3%) | <0.001 |
| Aspiration | 86 (8.3%) | 45 (10.0%) | 0.295 |
| Multiple Transfusion | 118 (11.4%) | 47 (10.5%) | 0.583 |
| Trauma | 80 (7.8%) | 32 (7.1%) | 0.672 |
| Pulmonary injury^{a} | 505 (49.0%) | 326 (72.6%) | <0.001 |
| Comorbidity, n (%) | | | |
| Diabetes | 282 (27.3%) | 78 (17.4%) | <0.001 |
| Liver cirrhosis/failure | 39 (3.8%) | 31 (6.9%) | 0.009 |
| History of alcohol abuse | 101 (9.8%) | 64 (14.2%) | 0.012 |

ARDS, acute respiratory distress syndrome;
APACHE, Acute Physiology and Chronic Health Evaluation;
SD, standard deviation.
^{a}Pneumonia, aspiration or pulmonary contusions are categorized as pulmonary injury. Sepsis from an extrapulmonary source, trauma without pulmonary contusions and multiple transfusions were categorized as extrapulmonary injury. Patients with both pulmonary and extrapulmonary injury were considered to have pulmonary injury.

TABLE 12
Polymorphisms detected in PI3 gene

| | | Chr20 nt | | | |
|---|---|---|---|---|---|
| No^{a} | SNP ID | position^{b} | Region | Alleles | MAF (n)^{c} |
| PI3-1 | novel | 43234809 | promoter | T > A | 0.136 (44) |
| PI3-2 | rs60717610 | 43235071 | promoter | —/TAATA | 0.214 (56) |
| PI3-3 | rs13044826 | 43235279 | promoter | G > A | 0.214 (56) |
| PI3-4 | rs56191952 | 43235901 | promoter | A > G | 0.214 (56) |
| PI3-5 | rs55767422 | 43235911 | promoter | A > G | 0.214 (56) |
| PI3-6 | rs56387543 | 43236018 | promoter | —/T | 0.214 (56) |
| PI3-7 | rs35476703 | 43236289 | promoter | C > G | 0.211 (52) |
| PI3-8 | rs35632684 | 43236303 | promoter | C > T | 0.211 (52) |
| PI3-9 | rs62208416 | 43236640 | promoter | G > A | 0.214 (56) |
| PI3-10 | rs41282752 | 43237020 | exon 1 | G > A, A15T | 0.018 (56) |
| PI3-11 | rs17333103 | 43237027 | exon 1 | C > T, T17M | 0.232 (56) |
| PI3-12 | rs17333180 | 43237122 | intron 1 | C > A | 0.214 (56) |
| PI3-13 | rs1983649 | 43237139 | intron 1 | A > T | 0.340 (56) |
| PI3-14 | rs6032040 | 43237728 | intron 1 | T > A | 0.108 (56) |
| PI3-15 | rs2664581 | 43237936 | exon 2 | A > C, T34P | 0.203 (54) |
| PI3-16 | rs34885285 | 43238203 | intron 2 | C > A | 0.203 (54) |
| PI3-17 | rs17424474 | 43238211 | intron 2 | C > A | 0.203 (54) |
| PI3-18 | rs17333381 | 43238333 | intron 2 | C > T | 0.203 (54) |
| PI3-19 | novel | 43238432 | exon 3 (3′UTR) | C > A | 0.018 (56) |
| PI3-20 | rs34412950 | 43238546 | exon 3 (3′UTR) | C > A | 0.214 (56) |
| PI3-21 | rs35869085 | 43238548 | exon 3 (3′UTR) | G > C | 0.214 (56) |
| PI3-22 | novel | 43238671 | 3′UTR | —/TCT | 0.214 (56) |
| PI3-23 | rs45461302 | 43238688 | 3′UTR | G > A | 0.196 (56) |
| PI3-24 | rs2267864 | 43238693 | 3′UTR | C > G | 0.203 (54) |

SNP, single nucleotide polymorphism;
nt, nucleotide;
MAF, Minor Allele Frequency;
UTR, untranslated region.
^{a}Polymorphisms are presented in chromosomal order and their position within the gene is indicated.
^{b}Chromosome position based on National Center for Biotechnology Information.
^{c}(n) is the number of chromosomes used in the estimation of MAF.

TABLE 13
SNPs genotyped in the current study

| | | | | Minor Allele Frequency | | | | |
|---|---|---|---|---|---|---|---|---|
| | | | Chr20 nt | All | | | | |
| SNP | Allele | Location | position^{a} | Subjects | ARDS | Controls | P^{b} | P^{c} |
| rs1983649 | A > T | intron 1 | 43237139 | 0.417 | 0.429 | 0.412 | 0.389 | 0.800 |
| rs6032040 | T > A | intron 1 | 43237728 | 0.170 | 0.161 | 0.173 | 0.456 | 0.183 |
| rs2664581 | A > C | exon 2 | 43237936 | 0.183 | 0.214 | 0.173 | 0.008 | 0.740 |

SNP, single nucleotide polymorphism;
nt, nucleotide:
ARDS, acute respiratory distress syndrome.
^{a}Chromosome position based on National Center for Biotechnology Information.
^{b}The probability of the chi-square test for comparison of allele frequencies.
^{c}The probability of the chi-square test for deviation from Hardy-Weinberg equilibrium in the control group.

TABLE 14
Association between PI3 genotypes and acute respiratory distress
syndrome (ARDS) risk

| | | | | Subjects with | | | Subjects with | | |
|---|---|---|---|---|---|---|---|---|---|
| | All subjects | | | pulmonary injury | | | extrapulmonary injury | | |
| | (n = 1480) | | | (n = 831) | | | (n = 649) | | |
| | Crude OR | Adjusted OR^{c} | | Crude OR | Adjusted OR^{c} | | Crude OR | Adjusted OR^{c} | |
| SNP | (95% CI) | (95% CI) | P | (95% CI) | (95% CI) | P | (95% CI) | (95% CI) | P |
| rs1983649 | | | | | | | | | |
| Additive^{a} | 1.07 | 1.09 | 0.324 | 0.97 | 0.95 | 0.635 | 1.29 | 1.37 | 0.034^{e} |
| | (0.91-1.25) | (0.92-1.29) | | (0.79-1.18) | (0.77-1.18) | | (0.98-1.70) | (1.02-1.84) | |
| Dominant^{b} | 1.08 | 1.10 | 0.449 | 0.97 | 0.98 | 0.880 | 1.26 | 1.35 | 0.187 |
| | (0.85-1.36) | (0.85-1.42) | | (0.73-1.31) | (0.71-1.34) | | (0.83-1.92) | (0.86-2.10) | |
| rs6032040 | | | | | | | | | |
| Additive^{a} | 0.92 | 0.96 | 0.759 | 0.83 | 0.86 | 0.303 | 1.08 | 1.09 | 0.670 |
| | (0.75-1.13) | (0.77-1.21) | | (0.64-1.07) | (0.65-1.14) | | (0.75-1.57) | (0.73-1.61) | |
| Dominant^{b} | 1.48 | 1.45 | 0.317 | 2.27 | 1.95 | 0.144 | 0.75 | 0.85 | 0.797 |
| | (0.75-2.93) | (0.70-2.98) | | (0.96-5.34) | (0.80-4.78) | | (0.24-2.35) | (0.24-2.96) | |
| rs2664581 | | | | | | | | | |
| Additive^{a} | 1.30 | 1.35 | 0.006^{d} | 1.24 | 1.19 | 0.193 | 1.48 | 1.65 | 0.004^{f} |
| | (1.07-1.58) | (1.09-1.67) | | (0.97-1.60) | (0.91-1.58) | | (1.06-2.06) | (1.16-2.34) | |
| Dominant^{b} | 1.35 | 1.43 | 0.006^{d} | 1.34 | 1.32 | 0.082 | 1.42 | 1.64 | 0.023^{e} |
| | (1.07-1.70) | (1.11-1.83) | | (1.00-1.80) | (0.96-1.81) | | (0.94-2.13) | (1.07-2.52) | |

SNP, single nucleotide polymorphism;
OR, odds ratio;
CI, confidence interval.
^{a}In the additive model, OR were expressed per difference in the number of minor alleles.
^{b}In the dominant model, OR were expressed as heterozygotes and rare homozygotes compared with common homozygotes.
^{c}Ajusted for age, gender sex, APACHE III score, risk factors for ARDS, comorbidities (diabetes, liver cirrhosis/failure) and alcohol abuse.
^{d}FDR P = 0.018.
^{e}FDR P = 0.051.
^{f}FDR P = 0.012.

TABLE 15
Association between PI3 haplotypes and acute respiratory distress
syndrome (ARDS) risk

| | | | | | Subjects with | | | Subjects with | | |
|---|---|---|---|---|---|---|---|---|---|---|
| | | All subjects | | | pulmonary injury | | | extrapulmonary injury | | |
| | | (n = 1480) | | | (n = 831) | | | (n = 649) | | |
| | | Haplotype | | | Haplotype | | | Haplotype | | |
| | | Frequency | | | Frequency | | | Frequency | | |
| Haplotype | Haplotype | (ARDS/ | Adjusted OR | | (ARDS/ | Adjusted OR | | (ARDS/ | Adjusted OR | |
| Identification | Composition^{a} | Controls) | (95% CI)^{b} | P | Controls) | (95% CI)^{b} | P | Controls) | (95% CI)^{b} | P |
| Global test^{c} | | | | 0.060 | | | 0.373 | | | 0.096 |
| Hap1 | ATA (0-0-0) | 56.8/58.7 | 1.0 | — | 58.3/57.5 | 1.0 | — | 52.9/60.0 | 1.0 | — |
| Hap2 | TTC (1-0-1) | 21.2/17.2^{d} | 1.31 | 0.015 | 20.2/17.0 | 1.14 | 0.370 | 23.9/17.3 | 1.67 | 0.005 |
| | | | (1.05-1.64) | | | (0.86-1.51) | | | (1.16-2.40) | |
| Hap3 | TAA (1-1-0) | 17.2/15.9 | 0.99 | 0.096 | 15.8/18.6 | 0.87 | 0.353 | 16.2/15.9 | 1.17 | 0.447 |
| | | | (0.78-1.26) | | | (0.65-1.16) | | | (0.77-1.79) | |
| Hap4 | TTA (1-0-0) | 5.7/6.7 | 0.78 | 0.185 | 5.5/6.7 | 0.72 | 0.167 | 6.1/6.7 | 0.97 | 0.895 |
| | | | (0.54-1.13) | | | (0.46-1.14) | | | (0.51-1.79) | |
| Others^{e} | — | — | — | — | — | — | — | — | — | — |

OR, odds ratio;
CI, confidence interval.
^{a}Individual tSNPs alleles are presented in chromosomal order rs1983649, rs6032040 and rs2664581.
^{b}Adjusted for age, gender, APACHE III score, risk factors for ARDS, comorbidities, alcohol abuse, recent use of steroid.
^{c}Global test for haplotypes by likehood-ratio test (LRT), with 4 degrees of freedom.
^{d}Chi-square test for comparison of haplotypes frequencies between ARDS and controls was P = 0.009, FDR P = 0.036.
^{e}Haplotypes with frequency <0.01%, not included in the analysis.

TABLE 16
Risk factors for ARDS required for study inclusion and exclusion criteria
considered

| Risk factor | Definition |
|---|---|
| Sepsis | Known or suspected source of systemic infection plus at least two of the following: a) |
| | temperature >38° C. or <36° C.; b) heart rate >90 beats/min; c) respiratory rate >20 breaths/min |
| | or PaCO₂ <32 mmHg; d) WBC count >12,000/mm³, <4000/mm³, or >10% |
| | bandemia. |
| Septic shock | Fulfill requirements for sepsis plus one of the following: a) SBP <90 mmHg or a |
| | reduction of ≧40 mmHg from baseline for ≧30 mins, unresponsive to 500 mL of |
| | fluid resuscitation; b) need for vasopressors to maintain SBP ≧90 mmHg or within 40 mmHg |
| | of baseline. |
| Pneumonia | Fulfill two or more of the following: a) new airspace opacity on chest radiograph:. b) |
| | temperature >38.3° C. or <36.0° C., WBC >12,000/mm³ or <4000/mm³ or >10% |
| | bandemia; c) positive microbiological culture. |
| Aspiration | Defined as witnessed or documented aspiration event or the retrieval of gastric |
| | contents from the oropharynx, endotracheal tube, or bronchial tree. |
| Trauma | Defined as multiple fractures and/or pulmonary contusions. Multiple fractures are |
| | defined as a fracture of two long bones, an unstable pelvic fracture, or one long bone |
| | and a pelvic fracture. Pulmonary contusion is defined as airspace opacity on chest |
| | radiograph within 8 hrs of admission to the emergency room and evidence of blunt |
| | trauma to the chest, for example, fractured ribs or ecchymosis overlying airspace |
| | opacity. |
| Multiple | Defined as receiving ≧8 units of packed red blood cells within 24 hrs. |
| transfusion | |

Abbreviation:
WBC, white blood cell;
SBP, systolic blood pressure
Exclusion criteria

| | |
|---|---|
| 1. | Age < 18 |
| 2. | Diffuse alveolar hemorrhage |
| 3. | Chronic lung diseases other than COPD or asthma |
| 4. | Directive to withhold intubation |
| 5. | Immunosuppression not secondary to corticosteroid |
| 6. | Treatment with granulocyte colony-stimulating factor |

TABLE 17
Baseline characteristics between ARDS cases and controls with available
plasma samples

| | Subjects did not | Subjects | |
|---|---|---|---|
| | develop ARDS | developed ARDS | |
| | (n = 64) | (n = 148) | p |
| Age-yr, mean ± SD | 61 ± 17 | 61 ± 19 | 0.961 |
| Male | 38 (59.4%) | 35 (52.2%) | 0.411 |
| APACHE III score, | 72 ± 23 | 86 ± 20 | 0.0003 |
| mean ± SD | | | |
| Risk factors, n (%) | | | |
| Sepsis | 24 (37.5%) | 18 (26.9%) | 0.263 |
| Septic shock | 28 (43.8%) | 40 (59.7%) | 0.068 |
| Pneumonia | 25 (39.1%) | 52 (77.6%) | <0.0001 |
| Aspiration | 6 (9.4%) | 8 (11.9%) | 0.635 |
| Multiple transfusion | 7 (10.9%) | 5 (7.5%) | 0.491 |
| Trauma | 6 (9.4%) | 2 (3.0%) | 0.127 |
| Direct lung injury^{a} | 31 (48.4%) | 54 (80.6%) | 0.0001 |
| Comorbidities, n (%) | | | |
| Diabetes | 15 (23.4%) | 15 (28.4%) | 0.521 |
| Liver failure/cirrhosis | 3 (4.7%) | 6 (9.0%) | 0.334 |
| Corticosteroid treatment | 4 (6.3%) | 13 (19.4%) | 0.025 |
| before ICU admission, | | | |
| n (%)^{b} | | | |

Abbreviation:
ARDS, acute respiratory distress syndrome;
APACHE, Acute Physiology and Chronic Health Evaluation;
SD, standard deviation.
^{a}Pneumonia, aspiration or pulmonary contusions are categorized as pulmonary injury. Sepsis from an extrapulmonary source, trauma without pulmonary contusions and multiple transfusions were categorized as extrapulmonary injury. Patients with both pulmonary and extrapulmonary injury were considered to have pulmonary injury.
^{b}Patient received ≧300 mg of prednisone or its equivalent within 21 days or ≧15 mg prednisone a day or its equivalent prior to ICU admission.

TABLE 18
PI3 genotype frequencies and comparisons between acute respiratory
distress syndrome (ARDS) and controls, and between survivors and nonsurvivors of ARDS

| | | ARDS Development | | | ARDS 28-Day Mortality | | | ARDS 60-Day Mortality | | |
|---|---|---|---|---|---|---|---|---|---|---|
| | Overall | ARDS | Controls | | Survivors | Nonsurvivors | | Survivors | Nonsurvivors | |
| | (n = 1480) | (n = 449) | (n = 1031) | P | (n = 299) | (n = 150) | P | (n = 270) | (n = 179) | P |
| rs1983649 | | | | 0.672 | | | 0.653 | | | 0.129 |
| AA | 508 (34.32%) | 149 (33.18%) | 359 (34.82%) | | 95 (31.77%) | 54 (36.00%) | | 80 (29.63%) | 69 (38.55%) | |
| TA | 710 (47.97%) | 215 (47.88%) | 495 (48.01%) | | 147 (49.16%) | 68 (45.33%) | | 138 (51.11%) | 77 (43.02%) | |
| TT | 262 (17.70%) | 85 (18.93%) | 177 (17.17%) | | 57 (19.06%) | 28 (18.67%) | | 52 (19.26%) | 33 (18.44%) | |
| rs6032040 | | | | 0.521 | | | 0.948 | | | 0.913 |
| AA | 1027 (69.39%) | 315 (70.16%) | 712 (69.06%) | | 206 (70.55%) | 103 (69.13%) | | 189 (70.00%) | 126 (70.39%) | |
| AT | 405 (27.36%) | 123 (27.39%) | 282 (27.35%) | | 79 (27.05%) | 42 (28.19%) | | 75 (27.78%) | 48 (26.82%) | |
| TT | 48 (3.24%) | 11 (2.45%) | 37 (3.59%) | | 7 (2.40%) | 4 (2.68%) | | 6 (2.22%) | 5 (2.79%) | |
| rs2664581 | | | | 0.030 | | | 0.413 | | | 0.236 |
| AA | 984 (66.49%) | 277 (61.69%) | 707 (68.57%) | | 178 (59.53%) | 99 (66.00%) | | 158 (58.52%) | 119 (66.48%) | |
| AC | 444 (30%) | 152 (33.85%) | 292 (28.32%) | | 107 (35.79%) | 45 (30.00%) | | 99 (36.67%) | 53 (29.61%) | |
| CC | 52 (3.51%) | 20 (4.45%) | 32 (3.10%) | | 14 (4.68%) | 6 (4.00%) | | 13 (4.81%) | 7 (3.91%) | |

TABLE 19
Baseline characteristics between survivors and nonsurvivors of ARDS

| | ARDS 28-day mortality | | | ARDS 60-day mortality | | |
|---|---|---|---|---|---|---|
| | Nonsurvivors | Survivors | | Nonsurvivors | Survivors | |
| Characteristic | (n = 150) | (n = 299) | P | (n = 179) | (n = 270) | P |
| Age-yr mean ± SD | 67.84 ± 14.64 | 55.29 ± 18.60 | <0.0001 | 67.78 ± 14.90 | 53.98 ± 18.37 | <0.0001 |
| Male | 87 (58.00%) | 182 (60.87%) | 0.558 | 106 (59.22%) | 163 (60.37%) | 0.807 |
| APACHE III score, mean ± SD | 90.59 ± 22.84 | 70.78 ± 21.46 | <0.0001 | 89.13 ± 22.51 | 69.62 ± 21.38 | <0.0001 |
| Risk factors, n (%) | | | | | | |
| Sepsis | 32 (21.33%) | 85 (28.43%) | 0.106 | 44 (24.58%) | 73 (27.04%) | 0.562 |
| Septic shock | 105 (70.00%) | 162 (54.18%) | 0.0013 | 116 (64.80%) | 151 (55.93%) | 0.061 |
| Pneumonia | 108 (72.00%) | 194 (64.88%) | 0.130 | 127 (70.95%) | 175 (64.81%) | 0.175 |
| Aspiration | 17 (11.33%) | 28 (9.36%) | 0.512 | 20 (11.17%) | 25 (9.26%) | 0.508 |
| Multiple Transfusion | 14 (9.33%) | 33 (11.04%) | 0.578 | 20 (11.17%) | 27 (10.00%) | 0.691 |
| Trauma | 2 (1.33%) | 30 (10.03%) | 0.0007 | 2 (1.12%) | 30 (11.11%) | <0.0001 |
| Pulmonary injury^{a} | 111 (74.00%) | 215 (71.91%) | 0.640 | 130 (72.63%) | 196 (72.59%) | 0.994 |
| Comorbidity, n (%) | | | | | | |
| Diabetes | 30 (20.00%) | 48 (16.05%) | 0.298 | 32 (17.88%) | 46 (17.04%) | 0.818 |
| Liver cirrhosis/failure | 16 (10.67%) | 15 (5.02%) | 0.026 | 21 (11.73%) | 10 (3.70%) | 0.0010 |
| History of alcohol abuse | 23 (15.33%) | 41 (13.71) | 0.643 | 28 (15.64%) | 36 (13.33%) | 0.493 |

Abbreviation:
ARDS, acute respiratory distress syndrome;
APACHE, Acute Physiology and Chronic Health Evaluation;
SD, standard deviation.
^{a}Pneumonia, aspiration or pulmonary contusions are categorized as pulmonary injury. Sepsis from an extrapulmonary source, trauma without pulmonary contusions and multiple transfusions were categorized as extrapulmonary injury. Patients with both pulmonary and extrapulmonary injury were considered to have pulmonary injury.

TABLE 20
Cox proportional hazard analysis of association between genetic variants
of PI3 and 60-day survival in patients with acute respiratory distress syndrome
(ARDS)
60-day

| | | | Subjects with | | Subjects with | |
|---|---|---|---|---|---|---|
| | All ARDS | | extrapulmonary injury | | pulmonary injury | |
| | (n = 449) | | (n = 123) | | (n = 326) | |
| | HR^{a} (95% CI) | p | HR^{a} (95% CI) | p | HR^{a} (95% CI) | p |
| Genotype | | | | | | |
| rs1983649 | 1.03 (0.83-1.28) | 0.777 | 0.93 (0.62-1.39) | 0.716 | 1.04 (0.81-1.35) | 0.738 |
| rs6032040 | 1.01 (0.76-1.34) | 0.966 | 1.28 (0.75-2.17) | 0.365 | 0.87 (0.61-1.25) | 0.324 |
| rs2664581 | 1.02 (0.77-1.36) | 0.874 | 0.85 (0.52-1.38) | 0.517 | 1.07 (0.75-1.52) | 0.699 |
| Haplotype | | | | | | |
| Global test^{b} | χ² = 1.78 | 0.776 | χ² = 0.71 | 0.950 | χ² = 3.63 | 0.458 |
| Hap1 ATA | | 1 | | 1 | | 1 |
| Hap2 TTC | 1.02 (0.76-1.37) | 0.882 | 0.86 (0.50-1.48) | 0.581 | 1.08 (0.75-1.55) | 0.683 |
| Hap3 TAA | 0.93 (0.68-1.26) | 0.637 | 1.14 (0.63-2.05) | 0.671 | 0.85 (0.58-1.24) | 0.389 |
| Hap4 TTA | 1.20 (0.74-1.94) | 0.462 | 0.96 (0.35-2.58) | 0.929 | 1.56 (0.89-2.73) | 0.121 |

^{a}HR Hazard ratio. Adjusted for age, gender, APACHE III score, risk factors for ARDS, comorbidities (diabetes, liver cirrhosis/failure) and alcohol abuse. HR was expressed per difference in the number of minor alleles (additive model).
^{b}Global test for the haplotypes by likehood-rario test (LTR), with 4 degrees of freedom.

TABLE 21
ANOVA of plasma profiles between ARDS and
controls by PI3 polymorphism^{a}

| | PI3 | | | | | Mean | |
|---|---|---|---|---|---|---|---|
| | Geno- | Control | | ARDS | | (95% CI) | |
| | type^{b} | AC/CC | AA | AC/CC | AA | ng/ml | P^{c} |
| Control | AC/CC | NA | − | − | − | 95.1 | 0.001 |
| | | | | | | (69.5-130) | |
| | AA | | NA | − | − | 72.6 | |
| | | | | | | (58.2-90.5) | |
| ARDS | AC/CC | | | NA | + | 64.9 | |
| | | | | | | (47.2-89.3) | |
| | AA | | | | NA | 44.3 | |
| | | | | | | (35.2-55.8) | |

^{a}ANOVA using generalized linear model with adjustment of covariables, including age, gender, type of lung injury, pre-admission steroid use, septic shock, and APACHE III score on ICU admission. Pairwise comparison was corrected by Bonferroni correction for multiple comparisons. +, P < 0.05; P ≧ 0.05; NA, not applicable.
^{b}The genotypes of rs2664581.
^{c}P values of generalized linear model for all groups.

1. A method of predicting whether a subject is at high risk of developing Acute Respiratory Distress Syndrome (ARDS) the method comprising:
determining the amount of elafin present in a test sample from the subject; and
comparing the amount of elafin in the test sample to the amount of elafin present in a control sample,
wherein an increased amount of elafin in the test sample relative to the amount of elafin in the control sample indicates that the subject is at high risk of developing ARDS.

2. A method of monitoring clinical progress of a subject who has or is at high risk of developing Acute Respiratory Distress Syndrome (ARDS) the method comprising:
determining the amount of elafin present in a first test sample from the subject;
determining the amount of elafin present in a second test sample from the subject taken at a later time; and
comparing the amount of elafin in the first test sample to the amount of elafin present in the second test sample,
wherein a change in the amount of elafin between the first and the second test samples indicates a change in the clinical progression of ARDS.

3-9. (canceled)

10. A kit for determining the amount of elafin in a test sample from a subject who has or is at high risk of developing Acute Respiratory Distress Syndrome (ARDS) comprising:
(a) a reagent to determine the amount of elafin in a subject sample;
(b) instructions for use; and,
(c) optionally, a reagent for isolating a sample from the subject.

11. A method of treating Acute Respiratory Distress Syndrome (ARDS) in a subject comprising:
administering to a subject diagnosed with ARDS an effective amount of elafin such that the ARDS is treated.

12. (canceled)

13. A method for determining the predisposition of a subject to develop ARDS comprising determining whether the genome of the subject comprises at least one single nucleotide polymorphism (SNP) associated with an increased risk of ARDS, to thereby determine the predisposition of the subject to develop ARDS.

14. The method of claim 13, further comprising detecting a polymorphism in the elafin gene.

15-16. (canceled)

17. A diagnostic kit comprising an oligonucleotide that specifically detects a human elafin nucleotide polymorphism.

18-27. (canceled)

28. The method of claim 13, further comprising determining whether the genome of the subject comprises a first polymorphism which is in linkage disequilibrium (LD) with a second polymorphism in the elafin gene, wherein the presence of the first polymorphism indicates that the subject has a predisposition to develop ARDS.

29-30. (canceled)

31. A method of determining the predisposition of a subject to develop Acute Respiratory Distress Syndrome (ARDS) the method comprising determining whether the genome of the subject comprises haplotype Hap2 (TTC); wherein the presence of the haplotype indicates that the subject is at high risk of developing ARDS.

32. The method of any one of claims 1, 2 and 31 further comprising determining whether the genome of the subject comprises at least one single nucleotide polymorphism (SNP) associated with an increased risk of ARDS.

33-43. (canceled)

44. The method of claims 1 or 2, further comprising determining whether the genome of the subject comprises haplotype Hap2 (TTC); wherein the presence of the haplotype indicates that the subject has a predisposition to develop ARDS.

45. The method of claim 32, further comprising determining whether the genome of the subject comprises a first polymorphism which is in linkage disequilibrium (LD) with a second polymorphism in the elafin gene, wherein the presence of the first polymorphism indicates that the subject has a predisposition to develop ARDS.

46. The method of claim 1, further comprising
determining the amount of neutrophil elastase present in a test sample from the subject;
and
comparing the amount of neutrophil elastase in the test sample to the amount of neutrophil elastase present in a control sample,
wherein an increased amount of neutrophil elastase in the test sample relative to the amount of neutrophil elastase in the control sample indicates that the subject is at high risk of developing ARDS.

47. The method of claim 46, further comprising
determining the ratio elafin:neutrophil elastase present in the test sample from the subject; and
comparing the ratio of elafin:neutrophil elastase in the test sample from the subject to the ration of elafin:neutrophil elastase present a control sample, wherein a decreased ratio of elafin:neutorphil elastase in the test sample relative to the ratio of elafin:neutrophil elastase in the control sample indicates that the subject is at high risk of developing ARDS.

48. The method of claim 2, further comprising
determining the amount of neutrophil elastase in the first and second test samples from the subject; and
comparing the amount of neutrophil elastase in the first test sample to the amount of neutrophil elastase in the second test sample,
wherein a change in the amount of neutrophil elastase between the first and second test samples indicates a change in the clinical progression of ARDS.

49. The method of claim 48, further comprising
determining the ratio of elafin:neutrophil elastase in the first and second test samples from the subject; and
comparing the ratio of elafin:neutrophil elastase in the first test sample to the amount of neutrophil elastase in the second test sample,
wherein a change in the ratio of elafin:neutrophil elastase between the first and second test samples indicates a change in the clinical progression of ARDS.

50. The method of claim 2, wherein the subject has ARDS and is undergoing a treatment regimen.

51. The kit of claim 10, further comprising a reagent to determine the amount of neutrophil elastase in a subject sample.

52. The kit of claim 10, further comprising reagents for determining the ratio of elafin:neutrophil elastase present in a subject sample.

53. The kit of claim 10, further comprising a reagent for determining whether the genome of the subject comprises haplotype Hap2 (TTC).

54. The kit of claim 10, further comprising a reagent for detecting the presence of a polymorphism in the elafin gene.


SEQUENCE LISTING (37 sequences)

SEQ ID NO: 1 - DNA, 601 bp, Homo sapiens
ctgctgggtgtcaccccagtctgaccactgctcctgagagacttggagtggaggaagggggaagaaacaaatactcaagggaactctggtcctgtagaccaccccaaaaaaggaagagccttccaagagtgtagctcccagaggtgtaccttccctactcaggccatggtttgaggatgctgcagtaagcagtggatggacccagacccagaggaaagacatggcagctgaagcagaggcttactgggtataaatgtgggctcgtttcttcttttaacagttcctgttaaaggtcaagacmctgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgaggtgagcactagctggagaacgaggagacccctgaagacacaaaag

SEQ ID NO: 2 - DNA, 801 bp, Homo sapiens
aaagtaggaaaactcttgggacaatcagagatgatgtgatgtaatgtccattagttcttcctgtgaataatcctgagggaaagcccccaggtccctcccagaatggggtggatatttcccaatacagctaaggaattatcccttgtaaataccacagacccgcccctggagccaggccaagctggactgcataaagattggtatggccttagctcttagccaaacaccttcctgacaccatgagggccagcagcttcttgatcgtggtggtgttcctcatcgctgggacgctggttctagaggcagctgtcacgggaggtgagtgaacaggtgacctgctgggctgggttggactaaggggagaccctctgggacaccctgggccaggacagggagcactwctgaagcagtaggcagcactggagcccagatttcagctttctgttctttgccatcatattcagaaaaaataggactttggctggtggactccacgtgctttccacctcagtgactgagatatcaggactgtttgtggaagtaatgttggtatgtggccttggcctcagatgtcaatacctgtgcagaatgtgcaataaaataatgaactccaggattttaaaccttgggtgtggacacagtcccccgtttctctgccccataaaagcactggagtaatcagtactctaaaaggaggttaagaaacaacaagccttcaggaatcatgttgtttgaggacccccattttataaggagggaaccaaaaatgtagaaatgagtgagcaattgccaaggtaattc

SEQ ID NO: 3 - DNA, 801 bp, Homo sapiens
tgtgcaataaaataatgaactccaggattttaaaccttgggtgtggacacagtcccccgtttctctgccccataaaagcactggagtaatcagtactctaaaaggaggttaagaaacaacaagccttcaggaatcatgttgtttgaggacccccattttataaggagggaaccaaaaatgtagaaatgagtgagcaattgccaaggtaattcccagagccaggatggggctcaagtctcctagtatgtggctcagggttctttcctactccaatgcacttcctaacaaatgacaatgtgtcctcttcactgctgggtgtcaccccagtctgaccactgctcctgagagacttggagtggaggaagggggaagaaacaaatactcaagggaactctggtccwgtagaccaccccaaaaaaggaagagccttccaagagtgtagctcccagaggtgtaccttccctactcaggccatggtttgaggatgctgcagtaagcagtggatggacccagacccagaggaaagacatggcagctgaagcagaggcttactgggtataaatgtgggctcgtttcttcttttaacagttcctgttaaaggtcaagacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgact

SEQ ID NO: 4 - protein, 117 aa, Homo sapiens
MRASSFLIVVVFLIAGTLVLEAAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ

SEQ ID NO: 5 - protein, 117 aa, Pan troglodytes; identical to SEQ ID NO: 4

SEQ ID NO: 6 - protein, 117 aa, Gorilla gorilla gorilla
MRASSFLIVVVFLIAGTLVLEAAVTGVPVKGQDTVKGRVPFNGQDPVKGQVSVKGQDKVKAQEPIKGPVSTKPGSCPVILIRCAMLNPPNRCLKDTDCPGIKKCCEGSCGMACFVPQ

SEQ ID NO: 7 - protein, 117 aa, Pongo abelii
MRASSFLIVVVFLIAGTLVVEAAVTGVPVKGQDTVKGRVPFNGQDPVKGQVSVKGQDRVKGQGPVKGPVSTKPGFCPNILIRCAMLNPPNRCLKDTDCPGIKKCCEGSCGMACFVPQ

SEQ ID NO: 8 - protein, 117 aa, Macaca mulatta
MRASSFLIVVVFLIAGMLVVEAAVTGVPVKGQDTVKGRVPFNGQDPVKGQVSVKGQDRVKGQGPVKGPVSTKPGSCPNILIRCAMLNPPNRCLKDTDCPGIKKCCEGSCGMACMVPQ

SEQ ID NO: 9 - protein, 117 aa, Papio anubis
MRASSFLIVVVFLIAGMLVVEAAVTGVPVKGQDTVKGHVPFNGQDPVKGQVSVKGQDWVKGQGPVKGPVSTKPGSCPNILIRCAMLNPPNRCLKDTDCPGIKKCCEGSCGMACMVPQ

SEQ ID NO: 10 - protein, 117 aa, Colobus guereza
MRASSFLIVVVFLIAGMLVVQAAVTGVPVKGQDTVKGHVPFNGQDPVKGQVSVKGQDRVKGQGPVKGPVSTKPGSCPNILIRCAMLNPPNRCLKDTDCPGIKKCCEGSCGMACMVPQ

SEQ ID NO: 11 - protein, 117 aa, Chlorocebus aethiops
MRASSFLIVVVFLIAGMLVVEAAVKGVPVKGQDTDKGRVPFNGQDPVKGQVSVKGQDRVKGQGPVKGPVSTKPGSCPNILIRCAMLNPPNHCLKDTDCPGIKKCCEGSCGMACMVPQ

SEQ ID NO: 12 - protein, 117 aa, Aotus nancymaae
MRASSFLIAVVLLIAGMLVVEAAVTGVPVKGQDTVKGRVLVNGQDPVKGQVSVKGQDRVKGQGPAKGQASTKSGSCPKTLIRCAMLNPPNRCLKDTDCPGIKKCCEGSCGMACLDPQ

SEQ ID NO: 13 - protein, 117 aa, Saimiri boliviensis
MRASSFLIAVVLLIAGMLVVEAAVMGVPVKGQDTVKGHVLANGQDPVKGQVSVKGQDRVKGQGPVKVQASTKSGSCPKILIQCAMLNPPNRCLKDTDCPGIKKCCEGSCGMACLDPQ

SEQ ID NO: 14 - protein, 116 aa, Callithrix jacchus
MRASSFLIAVVLLIAGMLVVEAAVMGVPVLTDTVKGHVLVNGQDPVKGQVSVKGQDKVKGQGPVKGQASIKSGSCPKTLIRCAMLNPPNRCLKDTDCPGIKKCCEGSCGMACLDPR

SEQ ID NO: 15 - protein, 117 aa, Otolemur garnettii
MRTSSFLVLVVLFILGTLAVEAAVKGVSAKGQGTGKGRVPLKGQDPLKGQVSGKDVEPVKGKGPVRGPGSTKSGSCPNILIRCAMLNPPNHCLSDTQCPGTKKCCEGSCGKRCMDPQ

SEQ ID NO: 16 - protein, 113 aa, Lemur catta
MRTSSFLVLAVFLILGMLAVEAAVRGVPVKGQGTGKGHVPVKGQDPLKGKAEDLVKGQDPVKRPGLTKPGSCPNILIRCAMLNPPNRCGSDGECPGSKKCCEGSCGRACLNPQ

SEQ ID NO: 17 - protein, 117 aa, Equus caballus
MRSSSFLILVVFLVLGTLAAQAAVAGVPPKGQGTDKGHALVKGRDPVRGQDPVKGQDSVKAQSLRKVPIVAKPGLCPKILIRCAMLNPPNRCLRDTECPGAKKCCVGSCGRVCMDPK

SEQ ID NO: 18 - protein, 107 aa, Canis familiaris
MRPSSFLVLAVFLILGTLAAQAAVTGVLPKGSGTDRGRVPVKGQDPVRGRDPVKAKGPVSTKPGSCPPILIQCAMLNPPNRCLSDTECPGARKCCKGPCGLACLQPQ

SEQ ID NO: 19 - DNA, 1001 bp, Homo sapiens
  modified_base (501)..(501): a, c, t, g, unknown or other
tgagctgagattgtgccattgaactccagcctgggtgacaagagcgaaattccatctcaaaaaaaaaaaaaaaaaaaaaaggagtattaaaaatgggtcagcttcaccagttattaggaaagtgtaaattcaaatcacaggaaatcccacaagatgtccacctgaatggctaatgaaaaggcaaatcatatgaggcattggtgagattatggagcaaatggaatgctcacacattgctgatagcagtgtgaattggtacaatgatgttggaaaattgattgcagtatttatgacagctggatgtatctatactctgtgatctgtactctgtgatccagcaattccattcctaaccatatatccaaaataaatgcatacctatgttctgcaagagatatgtacaagaatattcatagcaacactataatagctaaaacctagaaatatccaatgtatattgacagtagaatgaatatataacatttttggtatttttcatangaatactgtacagcagtgaaaatgatgaactacaggtgtagacttcaaaatggatgactctcaaaaatataatgttgagcaacataagcaggacatatgaacactataccttagatttcatttacatacaaattgaaaaagtatactaaactgaatatatatttcagaatacagacagaagtggcataactataaaggaaagcaagggctaattaccataaaatcttaggaagcagttatctctggggaggaaggagaatgaagcaactgaggagaggcatggagcatagatatagcaaatattctcctcctttacctggcagctcattttaatgctattctttaaagaatataggtattttctgtgagctttttatttgtttgtatggcttattgtacaatgagcttttgtgtaaggtagccaaaggcttccacagtctgacattcgtttatgtctccctcagtttcaggcttggtgggcatgtggtcagagtctaaat

SEQ ID NO: 20 - DNA, 874 bp, Homo sapiens
tatggagcaaatggaatgctcacacattgctgatagcagtgtgaattggtacaatgatgttggaaaattgattgcagtatttatgacagctggatgtatctatactctgtgatctgtactctgtgatccagcaattccattcctaaccatatatccaaaataaatgcatacctatgttctgcaagagatatgtacaagaatattcatagcaacactataatagctaaaacctagaaatatccaatgtatattgacagtagaatgaatatataacatttttggtatttttcatagaatactgtacagcagtgaaaatgatgaactacaggtgtagacttcaaaatggatgactctcaaaaatataatgttgagcaacataagcaggacatatgaacactataccttagatttcatttacatacaaattgaaaaagtatactaaactgaatatatatttcagaatacagacagaagtggcataactataaaggaaagcaaggrctaattaccataaaatcttaggaagcagttatctctggggaggaaggagaatgaagcaactgaggagaggcatggagcatagatatagcaaatattctcctcctttacctggcagctcattttaatgctattctttaaagaatataggtattttctgtgagctttttatttgtttgtatggcttattgtacaatgagcttttgtgtaaggtagccaaaggcttccacagtctgacattcgtttatgtctccctcagtttcaggcttggtgggcatgtggtcagagtctaaatcgacaaggggtggggatttcaccagctgaattagtgtccaataattagtttgttaccttccctgccttaactccacctcaa

SEQ ID NO: 21 - DNA, 675 bp, Homo sapiens
ttcgtttatgtctccctcagtttcaggcttggtgggcatgtggtcagagtctaaatcgacaaggggtggggatttcaccagctgaattagtgtccaataattagtttgttaccttccctgccttaactccacctcaactcagtgactttcttaactcagattctgtccctgacaatacccaacttcaggatattcatttctattactcagaacttttggtaacaatgacagaatagtaagtcagactagctgaagcagagggctaatttattgcctcataacccaaccatgagaagggtgtgtgaagaactgctctcaaggtaaaggagggaaacctggctgtgtcatctgaatcttctctcaccatcacttctctccttgtctcrgtgtattagagtcgtttttctcagacaattgatctccacccccagacttagataaccacttctggaattccctggttcatatatatcaccttttccagaagaaggaacccccgttttcccctttcattactcattcaaataaaatatgttaatagaccagaccaagtctcacacacaacacacacacacacactcacacacacacacacacacacacacccctacctgtggtcaggggtcaggctttcatgattagcagtccccattagactcacaagttgatggttca

SEQ ID NO: 22 - DNA, 675 bp, Homo sapiens
ttcgtttatgtctccctcagtttcaggcttggtgggcatgtggtcagagtctaaatcgacaaggggtggggatttcaccagctgaattagtgtccaataattagtttgttaccttccctgccttaactccacctcaactcagtgactttcttaactcagattctgtccctgacaatacccaacttcaggatattcatttctattactcagaacttttggtaacaatgacagaatagtaagtcagactagctgaagcagagggctaatttattgcctcataacccaaccatgagaagggtgtgtgaagaactgctctcaaggtaaaggagggaaacctggctgtgtcatctgaatcttctctcaccatcacttctctccttgtctcagtgtattagrgtcgtttttctcagacaattgatctccacccccagacttagataaccacttctggaattccctggttcatatatatcaccttttccagaagaaggaacccccgttttcccctttcattactcattcaaataaaatatgttaatagaccagaccaagtctcacacacaacacacacacacacactcacacacacacacacacacacacacccctacctgtggtcaggggtcaggctttcatgattagcagtccccattagactcacaagttgatggttca

SEQ ID NO: 23 - DNA, 676 bp, Homo sapiens
  modified_base (426)..(426): a, c, t, g, unknown or other
ccagctgaattagtgtccaataattagtttgttaccttccctgccttaactccacctcaactcagtgactttcttaactcagattctgtccctgacaatacccaacttcaggatattcatttctattactcagaacttttggtaacaatgacagaatagtaagtcagactagctgaagcagagggctaatttattgcctcataacccaaccatgagaagggtgtgtgaagaactgctctcaaggtaaaggagggaaacctggctgtgtcatctgaatcttctctcaccatcacttctctccttgtctcagtgtattagagtcgtttttctcagacaattgatctccacccccagacttagataaccacttctggaattccctggttcatatatatcaccttttccagaagaaggaacccccgtttncccctttcattactcattcaaataaaatatgttaatagaccagaccaagtctcacacacaacacacacacacacactcacacacacacacacacacacacacccctacctgtggtcaggggtcaggctttcatgattagcagtccccattagactcacaagttgatggttcaaagagacaacaattcttcacaagaaacagggtgctgttcaaagaagtggggaggcatgctggacacacacactgtaga

SEQ ID NO: 24 - DNA, 601 bp, Homo sapiens
accttttccagaagaaggaacccccgttttcccctttcattactcattcaaataaaatatgttaatagaccagaccaagtctcacacacaacacacacacacacactcacacacacacacacacacacacacccctacctgtggtcaggggtcaggctttcatgattagcagtccccattagactcacaagttgatggttcaaagagacaacaattcttcacaagaaacagggtgctgttcaaagaagtggggaggcatgctggacacacacactgtagatgtccaatgtatacatgatasatgttttctactactttctgattattttctccctaccactgtgatttcaagggcatcatccctcttcagacctagaattccaccttccaattactttcttgatgcatctgaaaggccgtctctgaaacagcaaagtgcaataattagtcattatgtattcaaggcaaatttgtcttacagagttttttgcaggaccagggaagaaggaaggaaatgcccagtttgatgctgggagtggtaaaatgataaagtagatctgggtggggtttgtagcaccagagcataatggagaaacacctt

SEQ ID NO: 25 - DNA, 601 bp, Homo sapiens
aaggaacccccgttttcccctttcattactcattcaaataaaatatgttaatagaccagaccaagtctcacacacaacacacacacacacactcacacacacacacacacacacacacccctacctgtggtcaggggtcaggctttcatgattagcagtccccattagactcacaagttgatggttcaaagagacaacaattcttcacaagaaacagggtgctgttcaaagaagtggggaggcatgctggacacacacactgtagatgtccaatgtatacatgatacatgttttctactaytttctgattattttctccctaccactgtgatttcaagggcatcatccctcttcagacctagaattccaccttccaattactttcttgatgcatctgaaaggccgtctctgaaacagcaaagtgcaataattagtcattatgtattcaaggcaaatttgtcttacagagttttttgcaggaccagggaagaaggaaggaaatgcccagtttgatgctgggagtggtaaaatgataaagtagatctgggtggggtttgtagcaccagagcataatggagaaacaccttggttttgtaatcaa

SEQ ID NO: 26 - DNA, 501 bp, Homo sapiens
gatgcatctgaaaggccgtctctgaaacagcaaagtgcaataattagtcattatgtattcaaggcaaatttgtcttacagagttttttgcaggaccagggaagaaggaaggaaatgcccagtttgatgctgggagtggtaaaatgataaagtagatctgggtggggtttgtagcaccagagcataatggagaaacaccttggttttgtaatcaagactggatctaccagtgacttgctgaataaccttcgrtgattcctttctcttcttgggtctcactgtatttcaaaacatgaagaatttcattgtaatgttacctaataagtgagccagcacttctactctgtgagaaagtaggaaaactcttgggacaatcagagatgatgtgatgtaatgtccattagttcttcctgtgaataatcctgagggaaagcccccaggtccctcccagaatggggtggatatttcccaatacagctaaggaattatcccttgtaaatac

SEQ ID NO: 27 - DNA, 801 bp, Homo sapiens
gacttgctgaataaccttcggtgattcctttctcttcttgggtctcactgtatttcaaaacatgaagaatttcattgtaatgttacctaataagtgagccagcacttctactctgtgagaaagtaggaaaactcttgggacaatcagagatgatgtgatgtaatgtccattagttcttcctgtgaataatcctgagggaaagcccccaggtccctcccagaatggggtggatatttcccaatacagctaaggaattatcccttgtaaataccacagacccgcccctggagccaggccaagctggactgcataaagattggtatggccttagctcttagccaaacaccttcctgacaccatgagggccagcagcttcttgatcgtggtggtgttcctcatcrctgggacgctggttctagaggcagctgtcacgggaggtgagtgaacaggtgacctgctgggctgggttggactaaggggagaccctctgggacaccctgggccaggacagggagcacttctgaagcagtaggcagcactggagcccagatttcagctttctgttctttgccatcatattcagaaaaaataggactttggctggtggactccacgtgctttccacctcagtgactgagatatcaggactgtttgtggaagtaatgttggtatgtggccttggcctcagatgtcaatacctgtgcagaatgtgcaataaaataatgaactccaggattttaaaccttgggtgtggacacagtcccccgtttctctgccccataaaagcactggagtaatcag

SEQ ID NO: 28 - DNA, 801 bp, Homo sapiens
tgaataaccttcggtgattcctttctcttcttgggtctcactgtatttcaaaacatgaagaatttcattgtaatgttacctaataagtgagccagcacttctactctgtgagaaagtaggaaaactcttgggacaatcagagatgatgtgatgtaatgtccattagttcttcctgtgaataatcctgagggaaagcccccaggtccctcccagaatggggtggatatttcccaatacagctaaggaattatcccttgtaaataccacagacccgcccctggagccaggccaagctggactgcataaagattggtatggccttagctcttagccaaacaccttcctgacaccatgagggccagcagcttcttgatcgtggtggtgttcctcatcgctgggaygctggttctagaggcagctgtcacgggaggtgagtgaacaggtgacctgctgggctgggttggactaaggggagaccctctgggacaccctgggccaggacagggagcacttctgaagcagtaggcagcactggagcccagatttcagctttctgttctttgccatcatattcagaaaaaataggactttggctggtggactccacgtgctttccacctcagtgactgagatatcaggactgtttgtggaagtaatgttggtatgtggccttggcctcagatgtcaatacctgtgcagaatgtgcaataaaataatgaactccaggattttaaaccttgggtgtggacacagtcccccgtttctctgccccataaaagcactggagtaatcagtactcta

SEQ ID NO: 29 - DNA, 501 bp, Homo sapiens
aattatcccttgtaaataccacagacccgcccctggagccaggccaagctggactgcataaagattggtatggccttagctcttagccaaacaccttcctgacaccatgagggccagcagcttcttgatcgtggtggtgttcctcatcgctgggacgctggttctagaggcagctgtcacgggaggtgagtgaacaggtgacctgctgggctgggttggactaaggggagaccctctgggacaccctgggmcaggacagggagcacttctgaagcagtaggcagcactggagcccagatttcagctttctgttctttgccatcatattcagaaaaaataggactttggctggtggactccacgtgctttccacctcagtgactgagatatcaggactgtttgtggaagtaatgttggtatgtggccttggcctcagatgtcaatacctgtgcagaatgtgcaataaaataatgaactccaggattttaaaccttgggtgtg

SEQ ID NO: 30 - DNA, 801 bp, Homo sapiens
ggtttgaggatgctgcagtaagcagtggatggacccagacccagaggaaagacatggcagctgaagcagaggcttactgggtataaatgtgggctcgtttcttcttttaacagttcctgttaaaggtcaagacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgaggtgagcactagmtggagaacgaggagacccctgaagacacaaaagaaggctgagcggtggggaagcatcccaggttggtgggagggaggttgtgggaggtgacagaaagactgggagactgaggggtctgagaggctataaccagagtgcctagaaggatgatctgtcttcctcactgcctctgagtgctttgatgtgctgactctcacctctgatactcttctcttccacagagggagccggtccttgctgcacctgtgccgtccccagagctacaggccccatctggtcctaagtccctgctgcccttccccttcccacactgtccattcttcctcccattcaggatgcccacggctggagctgcctctctcatccactttccaataaagagttccttctgctccacttg

SEQ ID NO: 31 - DNA, 601 bp, Homo sapiens
aacagttcctgttaaaggtcaagacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgaggtgagcactagctggagaamgaggagacccctgaagacacaaaagaaggctgagcggtggggaagcatcccaggttggtgggagggaggttgtgggaggtgacagaaagactgggagactgaggggtctgagaggctataaccagagtgcctagaaggatgatctgtcttcctcactgcctctgagtgctttgatgtgctgactctcacctctgatactcttctcttccacagagggagccggtccttgctgcacctgtgccgtccccagagctacaggccccatctggtcctaagtccctgctgcccttccccttccca

SEQ ID NO: 32 - DNA, 801 bp, Homo sapiens
gacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgaggtgagcactagctggagaacgaggagacccctgaagacacaaaagaaggctgagcggtggggaagcatcccaggttggtgggagggaggttgtgggaggtgacagaaagactgggagactgaggggtctgagaggctataaycagagtgcctagaaggatgatctgtcttcctcactgcctctgagtgctttgatgtgctgactctcacctctgatactcttctcttccacagagggagccggtccttgctgcacctgtgccgtccccagagctacaggccccatctggtcctaagtccctgctgcccttccccttcccacactgtccattcttcctcccattcaggatgcccacggctggagctgcctctctcatccactttccaataaagagttccttctgctccacttgtttctggttcctatgacttctgggctcctggatgctttggggaaatggatgtagaattgggacttcttctctccagtgaagaggggaaacggtcccatggtgaaagagagcagggcggggggaggaaaca

SEQ ID NO: 33 - DNA, 601 bp, Homo sapiens
cggtggggaagcatcccaggttggtgggagggaggttgtgggaggtgacagaaagactgggagactgaggggtctgagaggctataaccagagtgcctagaaggatgatctgtcttcctcactgcctctgagtgctttgatgtgctgactctcacctctgatactcttctcttccacagagggagccggtccttgctgcacctgtgccgtccccagagctacaggccccatctggtcctaagtccctgctgcccttccccttcccacactgtccattcttcctcccattcaggatgcccamggctggagctgcctctctcatccactttccaataaagagttccttctgctccacttgtttctggttcctatgacttctgggctcctggatgctttggggaaatggatgtagaattgggacttcttctctccagtgaagaggggaaacggtcccatggtgaaagagagcagggcggggggaggaaacaggaggcacattgctagggcttcatattacaaatccaataatcagccccagtgctgccagctccttttcaccaggaaatggaaatatggaaatcttgcctttggccaaggacca

SEQ ID NO: 34 - DNA, 601 bp, Homo sapiens
gtggggaagcatcccaggttggtgggagggaggttgtgggaggtgacagaaagactgggagactgaggggtctgagaggctataaccagagtgcctagaaggatgatctgtcttcctcactgcctctgagtgctttgatgtgctgactctcacctctgatactcttctcttccacagagggagccggtccttgctgcacctgtgccgtccccagagctacaggccccatctggtcctaagtccctgctgcccttccccttcccacactgtccattcttcctcccattcaggatgcccacgsctggagctgcctctctcatccactttccaataaagagttccttctgctccacttgtttctggttcctatgacttctgggctcctggatgctttggggaaatggatgtagaattgggacttcttctctccagtgaagaggggaaacggtcccatggtgaaagagagcagggcggggggaggaaacaggaggcacattgctagggcttcatattacaaatccaataatcagccccagtgctgccagctccttttcaccaggaaatggaaatatggaaatcttgcctttggccaaggaccagg

SEQ ID NO: 35 - DNA, 801 bp, Homo sapiens
aggtgacagaaagactgggagactgaggggtctgagaggctataaccagagtgcctagaaggatgatctgtcttcctcactgcctctgagtgctttgatgtgctgactctcacctctgatactcttctcttccacagagggagccggtccttgctgcacctgtgccgtccccagagctacaggccccatctggtcctaagtccctgctgcccttccccttcccacactgtccattcttcctcccattcaggatgcccacggctggagctgcctctctcatccactttccaataaagagttccttctgctccacttgtttctggttcctatgacttctgggctcctggatgctttggggaaatggatgtagaattgggacttcttctctccagtgaagaggrgaaacggtcccatggtgaaagagagcagggcggggggaggaaacaggaggcacattgctagggcttcatattacaaatccaataatcagccccagtgctgccagctccttttcaccaggaaatggaaatatggaaatcttgcctttggccaaggaccaggaaaaacacaaaacacaaaaaataaataaacattacataaaccataagcaggtcttttttttccaatccaccatcagtcctgacctggctactgtcggccttaccagtgtgaccagccaacccttatggctgcaactacagctccccacttccttctcattaatgtttcccaggaatctatatcttttcaacaccacatagaaaggatgatgttacagatgctgcgaaactcctgctcttt

SEQ ID NO: 36 - DNA, 601 bp, Homo sapiens
actctcacctctgatactcttctcttccacagagggagccggtccttgctgcacctgtgccgtccccagagctacaggccccatctggtcctaagtccctgctgcccttccccttcccacactgtccattcttcctcccattcaggatgcccacggctggagctgcctctctcatccactttccaataaagagttccttctgctccacttgtttctggttcctatgacttctgggctcctggatgctttggggaaatggatgtagaattgggacttcttctctccagtgaagaggggaaasggtcccatggtgaaagagagcagggcggggggaggaaacaggaggcacattgctagggcttcatattacaaatccaataatcagccccagtgctgccagctccttttcaccaggaaatggaaatatggaaatcttgcctttggccaaggaccaggaaaaacacaaaacacaaaaaataaataaacattacataaaccataagcaggtcttttttttccaatccaccatcagtcctgacctggctactgtcggccttaccagtgtgaccagccaacccttatggctgcaactacagctccc

SEQ ID NO: 37 - protein, 6 aa, Artificial sequence
  source: /note=“Description of artificial sequence Synthetic consensus sequence”
GQDPVK